(19) United States
(12) Patent Application Publication
(10) Pub. No.: US 2019/0307975 A1
(43) Pub. Date: Oct. 10, 2019

(54) MEDICAMENT DISPENSER

(71) Applicant: LUPIN INC., Coral Springs, FL (US)

(72) Inventors: Abhishek GUPTA, Coral Springs, FL (US); Imran SHAIKH, Coral Springs, FL (US); Xian-Ming ZENG, Coral Springs, FL (US)

(21) Appl. No.: 16/462,929

(22) PCT Filed: Nov. 21, 2017
(86) PCT No.: PCT/US2017/062792

§ 371 Date: May 21, 2019

Related U.S. Application Data
(60) Provisional application No. 62/424,759, filed on Nov. 21, 2016.
(60) Provisional application No. 62/424,749, filed on Nov. 21, 2016.
(60) Provisional application No. 62/424,730, filed on Nov. 21, 2016.
(60) Provisional application No. 62/424,691, filed on Nov. 21, 2016.

(51) Int. Cl. A61M 15/00; A61M 15/08

(52) U.S. Cl. CPC A61M 15/0048; A61M 15/08; A61M 15/0025; A61M 15/0068

(57) ABSTRACT

A medicament dispenser for dispensing medicament comprising: a diskette assembly comprising: at least one medicament carrier carrying multiple distinct medicament portions; a medicament carrier advancing mechanism for actuating the distinct medicament portions; a flow channel; a lever; and an outer cover for said flow channel, wherein the outer cover is adapted to couple with the lever and the medicament carrier advancing mechanism.

### RELATED APPLICATIONS

This application is being filed on 21 Nov. 2017, as a PCT International patent application, and claims priority to at least U.S. Provisional Application No. 62/424,759, filed on Nov. 21, 2016; U.S. Provisional Application No. 62/424,749, filed on Nov. 21, 2016; U.S. Provisional Application No. 62/424,730, filed on Nov. 21, 2016; and U.S. Provisional Application No. 62/424,691, filed on Nov. 21, 2016. The entirety of each of the aforementioned applications is hereby incorporated herein by reference.

### FIELD

The present disclosure relates to a medicament dispenser for dispensing medicament. The dispensing mechanism can include three steps to operate the medicament dispenser: Open, Dispense and Close.

### BACKGROUND

### Description of the Related Art

Medicaments are administered to a patient by inhalation using dry powder dispenser devices. Such devices are often referred to as dry powder inhalers (DPI), and are used for the treatment and prophylaxis of respiratory diseases, including but not limited to asthma, chronic obstructive pulmonary disease (COPD) bronchitis and chest infection.

### SUMMARY

Generally described, dry powder inhalers carry medicament in reservoirs, capsules or blisters packages, and can otherwise carry medicament. The medicament carrier can include a blister strip containing a number of discrete doses of powdered medicament. Such devices can contain a mechanism of accessing these doses, either piercing means or means to peel a lid sheet away from a base sheet. The powdered medicament can then be accessed and inhaled. Such a mechanism may also be used for dispensing medicament in tablets, capsules, pellets or pucks form wherein peeling away the lid sheet from the base sheet reveals a particular medicament form such as tablet for removal and subsequent consumption, particularly for oral administration.

U.S. Pat. No. 5,873,360 discloses an inhalation device, wherein a cover is pivotal between an open position in which the mouthpiece is exposed and a closed position in which it is not. In operation, the user moves the cover to its open position and then presses on the finger tab of the lever to cause it to move as the lever pivots. The lever associated with the finger tab, actuates the gear mechanism as the finger tab is slided from end to end. Therefore, the blister strip is advanced, one blister is opened, and one dose of the dry powder medicament contained in the blister cavity, become ready for the inhalation. The cover is held stationary as the body is rotated anticlockwise. As the cover of the device is not directly connected to the lever mechanism, the user needs to first move the cover and then lever and thus more number of steps are required to operate the device.

U.S. Pat. No. 8,161,968 discloses a medicament dispenser for containing plural elongate form medicament carriers, wherein said medicament dispenser comprises a movable cover for the outlet which is movable from an at rest position, in which the cover covers the outlet, to a primed position and then an actuated position to uncover the outlet, and couples to the dispensing mechanism such that: movement of said cover from the primed position to the actuated position actuates one or more components of the dispensing mechanism, and movement of said cover from the at rest position to the primed position does not actuate said one or more components of the dispensing mechanism.

U.S. Pat. No. 8,746,242 discloses a medicament dispenser for use with at least one medicament carrier carrying multiple distinct medicament portions, said medicament dispenser comprising (a) a dispensing mechanism actuable for dispensing the distinct medicament portions carried by said at least one medicament carrier; (b) a mouthpiece; and (c) a cover for said mouthpiece, said cover being movably mounted to the dispenser for sequential movement from a first position, in which said mouthpiece is covered, to a second position, in which said mouthpiece is part-uncovered, to a third position in which said mouthpiece is uncovered; wherein said cover is adapted to couple with said dispensing mechanism such that movement of the cover from the first position to the second position does not result in actuation of the dispensing mechanism, but any further movement of the cover from the second position to the third position results in actuation of the dispensing mechanism.

In operation, it is potentially desirable that dispensing mechanism comprises minimum number of steps to operate medicament dispenser. It is also potentially desirable that dispensing mechanism offers a continuous movement of the outer cover and without any second/intermediate position during opening and closing processes so as to avoid any confusion to the user. Further, increase in number of steps will increase complexity and cost associated with development of inhalation device.

In order to overcome such problems, the present disclosure describes a medicament dispenser which is cost-effective and which is used for dispensing medicament in reliable and easy way. Thus, the user actuates the mechanism with minimum possible steps to advance the medicament carrier thereby causing the next discrete dose of medicament to be made available for dispensing.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the outer cover is adapted to couple with the lever and the medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing for actuating the distinct medicament portions; iii) a flow channel; iv) a lever; and b) an outer cover for said flow channel comprises at least two ridges.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein said outer cover being movably mounted to the dispenser for sequential movement from an initial position, in which said flow channel is covered, to a dispense position, in which said flow channel is uncovered.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein said outer cover is adapted to couple with said medicament carrier advancing mechanism such that the movement of the outer cover from an initial position rotates the lever to actuate the medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein a first ridge of the outer cover is rotated along with the lever from an initial position to a dispense position to actuate the medicament carrier advancing mechanism for dispensing the distinct medicament portions.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein a second ridge of the outer cover retracts the lever through rotation of the outer cover in reverse direction from a dispense position to an initial position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein the mechanism of actuation provides continuous movement of outer cover without any idling position between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein mechanism of actuation rotates a cuvette ratchet by discrete angular movement through spur gears.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein a cuvette rotates synchronously with a cuvette ratchet in one direction, thereby advancing the medicament carrier.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein the outer cover couples directly with medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the flow channel is covered by the outer cover in an initial position, and fully revealed when the outer cover is in a dispense position.

Some aspects of the disclosure relate to a medicament dispenser for dispensing medicament comprising: a) a diskette assembly comprising: i) at least one medicament carrier carrying multiple distinct medicament portions; ii) mechanism of actuation for dispensing the distinct medicament portions; iii) a flow channel; iv) a mouthpiece; v) a lever; and b) an outer cover for said mouthpiece comprises at least two ridges, wherein the mouthpiece arranged such that the flow channel is covered by the outer cover in its initial position, and fully revealed when the outer cover is in its dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a mouthpiece; v) a lever; and b) an outer cover for said mouthpiece comprises at least two ridges, wherein the mechanism of actuation provides continuous movement of the outer cover such that there is no intermediate position for the outer cover between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein the coupling of the outer cover with the medicament carrier advancing mechanism is adapted such the movement of the cover from an initial position to a dispense position results in actuation of dispensing mechanism without any idle movement of the outer cover.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein said dispenser is an inhalation device.

Some aspects of the disclosure relate to a medicament dispenser for dispensing a medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein the dispenser is used for dispensing medicament for the treatment of asthma, chronic obstructive pulmonary disease (COPD), bronchitis or chest infection.

Some aspects of the disclosure relate to a medicament dispenser for dispensing medicament comprising: a) a diskette assembly comprising: a medicament carrier; a flow channel; a medicament carrier advancing mechanism; and a lever; and b) an outer cover comprising a first ridge; and a second ridge, wherein the outer cover is movable form an at initial position, in which the outer cover covers said flow channel, to a dispense position to uncover the flow channel, and the movement of the outer cover coupled to said lever and said medicament carrier advancing mechanism by the first ridge and the second ridge that are arranged such that movement of the outer cover from the initial position to the dispense position results in rotation of the lever and actuation of the medicament carrier advancing mechanism by the first ridge of the outer cover thereby advances the medicament carrier to align that with the flow channel and through which a user can access the medicament from said flow channel, and movement of the outer cover from the dispensed position to the initial position results in retraction of the lever by the second ridge of the outer cover back to the initial position.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the outer cover is adapted to couple with the lever and the medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein said outer cover being movably mounted to the dispenser for sequential movement from an initial position, in which said flow channel is covered, to a dispense position, in which said flow channel is uncovered.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein said outer cover is adapted to couple with said medicament carrier advancing mechanism such that the movement of the outer cover from an initial position rotates the lever to actuate the medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the plunger of the outer cover is rotated along with the lever from an initial position to a dispense position to actuate the medicament carrier advancing mechanism for dispensing the distinct medicament portions.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the plunger of the outer cover retracts the lever through rotation of the outer cover in reverse direction from a dispense position to an initial position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises a plunger, wherein the mechanism of actuation provides continuous movement of outer cover without any idling position between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises a plunger, wherein mechanism of actuation rotates a cuvette ratchet by discrete angular movement through spur gears.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises a plunger, wherein a cuvette rotates synchronously with a cuvette ratchet in one direction, thereby advancing the medicament carrier.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises a plunger, wherein the outer cover couples directly with medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the flow channel is covered by the outer cover in an initial position, and fully revealed when the outer cover is in a dispense position.

Some aspects of the disclosure relate to a medicament dispenser for dispensing medicament comprising: a) a diskette assembly comprising: i) at least one medicament carrier carrying multiple distinct medicament portions; ii) mechanism of actuation for dispensing the distinct medicament portions; iii) a flow channel; iv) a mouthpiece; v) a lever; and b) an outer cover for said mouthpiece comprises a plunger, wherein the mouthpiece arranged such that the flow channel is covered by the outer cover in its initial position, and fully revealed when the outer cover is in its dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a mouthpiece; v) a lever; and b) an outer cover for said mouthpiece comprises a plunger, wherein the mechanism of actuation provides continuous movement of the outer cover such that there is no intermediate position for the outer cover between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the coupling of the outer cover with the medicament carrier advancing mechanism is adapted such the movement of the cover from an initial position to a dispense position results in actuation of dispensing mechanism without any idle movement of the outer cover.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein said dispenser is an inhalation device.

Some aspects of the disclosure relate to a medicament dispenser for dispensing a medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the dispenser is suitable for dispensing medicament for the treatment of asthma, chronic obstructive pulmonary disease (COPD), bronchitis or chest infection.

Some aspects of the disclosure relate to a medicament dispenser for dispensing medicament comprising: a) a diskette assembly comprising: a medicament carrier; a flow channel; a medicament carrier advancing mechanism; and a lever having a driver; and b) an outer cover comprising a plunger having a plunger nose; a plunger cover; and compression spring, wherein the outer cover is movable from an initial position, in which the outer cover covers said flow channel, to a dispense position to uncover the flow channel, and the movement of the outer cover coupled to the driver and the medicament carrier advancing mechanism by the plunger nose that are arranged such that movement of the outer cover from the initial position to the dispense position results in rotation of the lever and actuation of the medicament carrier advancing mechanism by the plunger nose of the outer cover thereby advances the medicament carrier to align that with the flow channel and through which a user can access the medicament from said flow channel, and movement of the outer cover from the dispensed position to the initial position results in retraction of the lever by the plunger nose of the outer cover back to the initial position.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel; and c) an inner cover.

Some aspects of the disclosure relate to a medicament dispenser for dispensing medicament comprising: a) a diskette assembly comprising: i) at least one medicament carrier carrying multiple distinct medicament portions; ii) a medicament carrier advancing for actuating the distinct medicament portions; iii) a flow channel; iv) a lever; b) an outer cover for said flow channel comprises at least two ridges; and c) an inner cover

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein said outer cover being movably mounted to the dispenser for sequential movement from an initial position, in which said flow channel is covered, to a dispense position, in which said flow channel is uncovered.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein said outer cover is adapted to couple with said medicament carrier advancing mechanism such that the movement of the outer cover from an initial position rotates the lever to actuate the medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein a first ridge of the outer cover is rotated along with the lever from an initial position to a dispense position to actuate the medicament carrier advancing mechanism for dispensing the distinct medicament portions.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein a second ridge of the outer cover retracts the lever through rotation of the outer cover in reverse direction from a dispense position to an initial position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges, and c) an inner cover, wherein the mechanism of actuation provides continuous movement of outer cover without any idling position between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges, and c) an inner cover, wherein mechanism of actuation rotates a cuvette ratchet by discrete angular movement through spur gears.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges; and c) an inner cover, wherein a cuvette rotates synchronously with a cuvette ratchet in one direction, thereby advancing the medicament carrier.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges; and c) an inner cover, wherein the outer cover couples directly with medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel; and c) an inner cover, wherein the flow channel is covered by the outer cover in an initial position, and fully revealed when the outer cover is in a dispense position.

Some aspects of the disclosure relate to a medicament dispenser for dispensing medicament comprising: a) a diskette assembly comprising: i) at least one medicament carrier carrying multiple distinct medicament portions; ii) mechanism of actuation for dispensing the distinct medicament portions; iii) a flow channel; iv) a mouthpiece; v) a lever; b) an outer cover for said mouthpiece comprises at least two ridges; and c) an inner cover, wherein the mouthpiece arranged such that the flow channel is covered by the outer cover in its initial position, and fully revealed when the outer cover is in its dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a mouthpiece; v) a lever; b) an outer cover for said mouthpiece comprises at least two ridges; and c) an inner cover, wherein the mechanism of actuation provides continuous movement of the outer cover such that there is no intermediate position for the outer cover between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein the coupling of the outer cover with the medicament carrier advancing mechanism is adapted such the movement of the cover from an initial position to a dispense position results in actuation of dispensing mechanism without any idle movement of the outer cover.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges; and c) an inner cover, wherein said dispenser is an inhalation device.

Some aspects of the disclosure relate to a medicament dispenser for dispensing a medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein the dispenser is suitable for dispensing medicament for the treatment of asthma, chronic obstructive pulmonary disease (COPD), bronchitis or chest infection.

Some aspects of the disclosure relate to a medicament dispenser for dispensing medicament comprising: a) a diskette assembly comprising: a medicament carrier; a flow channel; a medicament carrier advancing mechanism; and a lever having a bar; b) an outer cover comprising a first ridge; and a second ridge; and c) an inner cover comprising a protrusion and a groove; wherein the outer cover is movable form an at initial position, in which the outer cover covers said flow channel, to a dispense position to uncover the flow channel, and the movement of the outer cover coupled to the inner cover and the medicament carrier advancing mechanism via said bar by the first ridge and the second ridge that are arranged such that movement of the outer cover from the initial position to the dispense position results in rotation of the inner cover and the lever and actuation of the medicament carrier advancing mechanism by the first ridge of the outer cover thereby advances the medicament carrier to align that with the flow channel and through which a user can access the medicament from said flow channel, and movement of the outer cover from the dispensed position to the initial position results in retraction of the inner cover thereby the lever by the second ridge of the outer cover back to the initial position.

Some aspects of the disclosure relate to is a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel comprises, wherein said outer cover being movably mounted to the dispenser for sequential movement from an initial position, in which said flow channel is covered, to a dispense position, in which said flow channel is uncovered.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein said outer cover is adapted to couple with said medicament carrier advancing mechanism such that the movement of the outer cover from an initial position rotates the central gear and the idle gear to actuate the medicament carrier advancing mechanism.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a central gear; v) an idle gear; and b) an outer cover for said flow channel, wherein the mechanism of actuation provides continuous movement of outer cover without any idling position between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a central gear; v) an idle gear; and b) an outer cover for said flow channel, wherein a cuvette rotates synchronously with a cuvette ratchet in one direction, thereby advancing the medicament carrier.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a central gear; v) an idle gear; and b) an outer cover for said flow channel, wherein the outer cover is adapted to couple with the central gear and the medicament carrier advancing mechanism via said idle gear.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the flow channel is covered by the outer cover in an initial position, and fully revealed when the outer cover is in a dispense position.

Some aspects of the disclosure relate to a medicament dispenser for dispensing medicament comprising: a) a diskette assembly comprising: i) at least one medicament carrier carrying multiple distinct medicament portions; ii) mechanism of actuation for dispensing the distinct medicament portions; iii) a flow channel; iv) a mouthpiece; iv) a central gear; v) an idle gear; and b) an outer cover for said mouthpiece, wherein the mouthpiece arranged such that the flow channel is covered by the outer cover in its initial position, and fully revealed when the outer cover is in its dispense position.

Some aspects of the disclosure relate to a medicament dispenser for dispensing medicament comprising: a) a diskette assembly comprising: i) at least one medicament carrier carrying multiple distinct medicament portions; ii) a mechanism of actuation for dispensing the distinct medicament portions; iii) a flow channel; iv) a mouthpiece iv) a central gear; v) an idle gear; and b) an outer cover for said mouthpiece comprises, wherein the mechanism of actuation provides continuous movement of the outer cover such that there is no intermediate position for outer cover between an initial position and a dispense position.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the coupling of the outer cover with the medicament carrier advancing mechanism is adapted such the movement of the cover from an initial position to a dispense position results in actuation of dispensing mechanism without any idle movement of the outer cover.

Some aspects of the disclosure relate to a medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the dispenser is an inhalation device.

Some aspects of the disclosure relate to a medicament dispenser for dispensing a medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the dispenser is suitable for dispensing medicaments for the treatment of asthma, chronic obstructive pulmonary disease (COPD), bronchitis or chest infection.

Some aspects of the disclosure relate to a medicament dispenser for dispensing medicament comprising: a) a diskette assembly comprising: a medicament carrier; a flow channel; a medicament carrier advancing mechanism; a central gear; and an idle gear; and b) an outer cover, wherein the outer cover is movable form an at initial position, in which the outer cover covers said flow channel, to a dispense position to uncover the flow channel, and the movement of the outer cover coupled to said medicament carrier advancing mechanism by the central gear and the idle gear that are arranged such that movement of the outer cover from the initial position to the dispense position results in actuation of the medicament carrier advancing mechanism by the central gear and the idle gear thereby advances the medicament carrier to align that with the flow channel, and through which a user can access the medicament from said flow channel, and movement of the outer cover from the dispense position to the initial position results in rotation of the central gear and the idle gear without advancing the medicament carrier.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

FIGS. 1A-1C show different views of one embodiment of the medicament dispenser.

FIG. 2 shows a partial exploded view of the medicament dispenser shown in FIGS. 1A-1C.

FIGS. 3A-3C show inner components and working of the medicament dispenser shown in FIGS. 1A-1C.

FIG. 4 shows another partial exploded view of the medicament dispenser shown in FIGS. 1A-1C.

FIG. 5 shows the outer cover of the medicament dispenser shown in FIGS. 1A-1C.

FIGS. 6A and 6B show different views of the space for the electronics in the outer cover.

FIGS. 7A and 7B show different views of the diskette assembly of the medicament dispenser shown in FIGS. 1A-1C.

FIG. 8 shows the lever of the medicament dispenser shown in FIGS. 1A-1C.

FIGS. 9A-9C show the relative positions of the lever and the outer cover during the operation of the medicament dispenser shown in FIGS. 1A-1C.

FIGS. 10A-10C show the demonstration of the Open, Dispense, Close mechanism of the medicament dispenser shown in FIGS. 1A-1C.

FIG. 11 shows an exploded view of the medicament dispenser shown in FIGS. 1A-1C.

FIGS. 12A-12D show different views of an embodiment of a medicament dispenser.

FIGS. 13A-13D show different views of another embodiment of a medicament dispenser.

FIGS. 14A-14C show the components and working of the medicament dispenser shown in FIGS. 12A-12D.

FIG. 15 shows a partial exploded view of the medicament dispenser shown in FIGS. 12A-12D.

FIG. 16 shows a partial exploded view of the medicament dispenser shown in FIGS. 13A-13D.

FIG. 17 shows an outer cover of the medicament dispenser shown in FIGS. 12A-12D.

FIGS. 18A & 18B show an outer cover of the medicament dispenser shown in FIGS. 13A-13D.

FIGS. 19A & 19B show the different views of an embodiment of the outer cover end that is compatible with the medicament dispensers described herein, and FIG. 19C shows an embodiment of the plunger that is compatible with the medicament dispensers described herein.

FIG. 20 shows an embodiment of the plunger cover and compression spring that is compatible with the medicament dispensers described herein.

FIG. 21 shows an embodiment of the lever that is compatible with the medicament dispensers described herein

FIG. 22A shows an embodiment of a medicament dispenser without an outer cover. FIG. 22B shows the medicament dispenser with the outer cover shown in FIG. 17. FIG. 22C shows the medicament dispenser with the outer cover shown in FIGS. 18A & 18B.

FIGS. 23A-23C show the relative positions of the lever and the outer cover during the operation of the medicament dispenser shown in FIGS. 12A-12D.

FIGS. 24A-24C show the relative positions of the lever and the outer cover during the operation of the medicament dispenser with the electronics compartment shown in FIGS. 13A-13D.

FIGS. 25A-25C show the demonstration of the Open, Dispense, Close mechanism of the medicament dispenser shown in FIGS. 12A-12D.

FIG. 26 shows an exploded view of the medicament dispenser shown in FIGS. 12A-12D.

FIGS. 27A-27C show different views of an embodiment of the medicament dispenser.

FIG. 28 shows a partial exploded view of the medicament dispenser shown in FIGS. 27A-27C.

FIGS. 29A-29C show the internal components and workings of the medicament dispenser shown in FIGS. 27A-27C.

FIG. 30 shows a partial exploded view of the medicament dispenser shown in FIGS. 27A-27C.

FIGS. 31A-31C show an example of the outer cover and the respective features.

FIG. 32 shows an example of the inner cover and the respective features.

FIG. 33 shows an example of the lever and the respective features.

FIG. 34A show the medicament dispenser shown in FIGS. 27A-27C without the inner and outer covers, and FIG. 34B shows the medicament dispenser shown in FIGS. 27A-27C with the inner and outer covers.

FIGS. 35A-35C show the relative positions of the lever, inner cover and the outer cover during the operation of the medicament dispenser shown in FIGS. 27A-27C.

FIGS. 36A-36C show the demonstration of the Open, Dispense, Close mechanism of the medicament dispenser shown in FIGS. 27A-27C.

FIG. 37 shows an exploded view of the medicament dispenser shown in FIGS. 27A-27C.

FIGS. 38A-38E show the relative positions of the lever, inner cover and the outer cover during the operation of the medicament dispenser shown in FIGS. 27A-27C.

FIGS. 39A-39C show different views an embodiment of the medicament dispenser.

FIG. 40 shows an exploded view of the medicament dispenser shown in FIGS. 39A-39C.

FIG. 41 shows the internal mechanism of the medicament dispenser shown in FIGS. 39A-39C.

FIG. 42 shows a partial view of the internal mechanism of the medicament dispenser shown in FIGS. 39A-39C.

FIG. 43 shows a partial exploded view of the medicament dispenser shown in FIGS. 39A-39C.

FIGS. 44A and 44B show an example of an outer cover that can be used with the medicament dispenser shown in FIGS. 39A-39C.

FIG. 45 shows an example of a central gear of the medicament dispenser shown in FIGS. 39A-39C.

FIG. 46 shows an example of an idle gear of the medicament dispenser shown in FIGS. 39A-39C.

FIG. 47 shows an example of a base cover of the medicament dispenser shown in FIGS. 39A-39C.

FIG. 48 shows an example of a top cover of the medicament dispenser shown in FIGS. 39A-39C.

FIG. 49A shows the medicament dispenser shown in FIGS. 39A-39C without the outer cover, and FIG. 49B shows of the medicament dispenser shown in FIGS. 39A-39C with the outer cover.

FIGS. 50A-50D show the relative positions of the outer cover, blister cuvette and central gear during the operation of the medicament dispenser.

FIGS. 51A-51C show the demonstration of the Open, Dispense, Close mechanism of the medicament dispenser.

FIGS. 52A-52C show the advancement of the reel of blisters during the operation of the medicament dispenser.

FIGS. 53A-53C show different views of one embodiment of the medicament dispenser.

FIG. 54 shows an exploded view of the medicament dispenser shown in FIGS. 53A-53C.

FIGS. 55A-55C shows the demonstration of the Open, Dispense, Close mechanism of the medicament dispenser shown in FIGS. 53A-53C.

FIG. 56 shows the internal mechanism of the medicament dispenser shown in FIGS. 53A-53C.

FIG. 57 shows another view of the internal mechanism of the medicament dispenser shown in FIGS. 53A-53C.

FIGS. 58A-58D show view of the medicament dispenser shown in FIGS. 53A-53C containing two reels of blisters.

FIG. 59 shows view of the medicament dispenser shown in FIGS. 53A-53C containing one reel of blisters.

FIGS. 60A-60F show the advancement of the reel of blisters during the operation of the medicament dispenser.

FIG. 61 shows an example of inner view of a base cover of the medicament dispenser described herein.

FIG. 62 shows an example of outer view of a base cover of the medicament dispenser described herein.

FIG. 63 shows the outer cover of the medicament dispenser shown in FIGS. 53A-53C.

FIGS. 64A-64B show different views of the space for the electronics in the outer cover.

FIGS. 65A-65C show the different views of an embodiment of the blister cuvette that is compatible with the medicament dispenser described herein.

FIG. 66 shows an embodiment of the cuvette ratchet that is compatible with the medicament dispenser described herein.

FIG. 67 shows the blister cuvette and the cuvette ratchet that is compatible with the medicament dispenser described herein.

FIG. 68 shows an embodiment of the winder gear that is compatible with the medicament dispenser described herein.

FIG. 69 shows an embodiment of the winder ratchet that is compatible with the medicament dispenser described herein.

FIG. 70 shows an embodiment of the winder rotor that is compatible with the medicament dispenser described herein.

FIGS. 71A-71B show the different views of the winding assembly that is compatible with the medicament dispenser described herein.

FIGS. 72A-72B show the different views of the winding assembly that is compatible with the medicament dispenser described herein containing laminations of two reels of blisters.

FIGS. 73A-73B show the different views of the inner housing assembly that is compatible with the medicament dispenser described herein.

FIG. 74 shows another view of the inner housing assembly that is compatible with the medicament dispenser described herein containing deck separators.

FIG. 75 shows another view of the inner housing assembly that is compatible with the medicament dispenser described herein containing two reels of blisters.

FIGS. 76A-76B show views of the deck separators that is compatible with the medicament dispenser described herein

FIGS. 77A-77B show the different views of the wrapping wheel that is compatible with the medicament dispenser described herein.

FIG. 78 shows the interim gear of the medicament dispenser shown in FIGS. 53A-53C.

FIGS. 79A-79B show the wrapping wheel that is compatible with the medicament dispenser described herein containing delaminated base sheets of two reels of blisters.

FIG. 80 shows the dose ring of the medicament dispenser shown in FIGS. 53A-53C.

FIG. 81 shows the lever of the medicament dispenser shown in FIGS. 53A-53C.

FIG. 82 shows an example of inner view of a top cover of the medicament dispenser described herein.

FIG. 83 shows an example of outer view of a top cover of the medicament dispenser described herein.

FIGS. 84A-84B show the different views of an embodiment of the reel of blisters that is compatible with the medicament dispenser described herein.

### DETAILED DESCRIPTION

The present disclosure relates to a medicament dispenser for dispensing medicament. Particularly, the present disclosure relates to the medicament carrier advancing mechanism for a medicament dispenser. More particularly, the present disclosure relates to mechanism of actuation for a medicament dispenser.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the outer cover is adapted to couple with the lever and the medicament carrier advancing mechanism.

In some embodiments, an inhalation device for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the outer cover is adapted to couple with the lever and the medicament carrier advancing mechanism.

In some embodiments, medicament comprises one or more active pharmaceutical ingredients (APIs) and optionally, one or more pharmaceutically acceptable carriers. The active pharmaceutical ingredients and pharmaceutically acceptable carriers may be present in micronized, non-micronized form or mixtures thereof.

One or more active pharmaceutical ingredients (APIs) that can be used in the inventions selected from, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α.-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate). α4 integrin inhibitors e.g. (2S)-3-[4-({[4-(aminocarbonyl)-1-piperidinyl]carbonyl}oxy)phenyl]-2-[((2S+4-methyl-2-{[2-(2 methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

In some embodiments, the medicament may in aspects, be a mono-therapy (i.e. single active medicament containing) product or it may be a combination therapy (i.e. plural active medicaments containing) product. Suitable medicaments or medicament components of a combination therapy product are typically selected from the group consisting of anti-inflammatory agents (for example a corticosteroid particularly inhaled corticosteroid (ICS) or an NSAID), anticholinergic agents (for example, an M₁, M₂, M₁/M₂ or M₃ receptor antagonist particularly long-acting muscarinic antagonist (LAMA), other β₂-adrenoreceptor agonists particularly long-acting β₂-agonist (LABA), anti-infective agents (e.g. an antibiotic or an antiviral), and antihistamines. All suitable combinations are envisaged. The present invention also provides combination therapy product typically double or triple LAMA, LABA, ICS combinations.

Suitable anti-inflammatory agents include corticosteroids and NSAIDs. Suitable corticosteroids are those oral and inhaled corticosteroids and their pro-drugs which have anti-inflammatory activity. Examples include methyl prednisolone, prednisolone, dexamethasone, fluticasone propionate, fluticasone furoate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α, 9α-difluoro-11β-hydroxy-16α-methyl-3-oxo-17α-propionyloxy-androsta-1,4-diene-17β-carbothioic acid S-(2-oxo-tetrahydro-furan-35-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126. Preferred corticosteroids include fluticasone propionate, fluticasone furoate 6α,9α-difluoro-11β-hydroxy-16α-methyl-17α-[(-4-methyl-1,3-thiazole-5-carbonyl)oxy]-3-oxo-androsta-1,4-diene-170-carbothioic acid S-fluoromethyl ester and 6α, 9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Suitable NSAIDs include cromolyns (e.g. sodium cromoglycate, nedocromil sodium), phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, β₂integrin antagonists and adenosine receptor agonists or antagonists (e.g. adenosine 2a agonists), cytokine antagonists (e.g. chemokine antagonists) or inhibitors of cytokine synthesis. Suitable other β₂-adrenoreceptor agonists include salmeterol (e.g. as the xinafoate), salbutamol (e.g. as the sulphate or the free base), formoterol (e.g. as the fumarate), vilanterol (e.g. as the trifenatate), fenoterol or terbutaline and salts thereof.

Suitable phosphodiesterase 4 (PDE4) inhibitors include compounds that are known to inhibit the PDE4 enzyme or which are discovered to act as a PDE4 inhibitor, and which are only PDE4 inhibitors, not compounds which inhibit other members of the PDE family as well as PDE4. Generally it is preferred to use a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity. For the purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

A method for determining IC₅₀s ratios is set out in U.S. Pat. No. 5,998,428 which is incorporated herein in full by reference as though set out herein. See also PCT application WO 00/51599 for another description of said assay.

Suitable PDE4 inhibitors include those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1 nM of [³H]R-rolipram to a form of PDE4 which binds rolipram with a high affinity over the IC₅₀ value for inhibiting the PDE4 catalytic activity of a form which binds rolipram with a low affinity using 1 muM[³H]-cAMP as the substrate.

Most suitable are those PDE4 inhibitors which have an IC₅₀ ratio of greater than 0.5, and particularly those compounds having a ratio of greater than 1.0. Preferred compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]; these are examples of compounds which bind preferentially to the low affinity binding site and which have an IC₅₀ ratio of 0.1 or greater.

Other suitable medicament compounds include: cis-4-cyano-4-[3-(cyclopentyloxy)-4-methoxyphenyl]cyclohexane-1-carboxylic acid (also known as cilomalast) disclosed in U.S. Pat. No. 5,552,438 and its salts, esters, pro-drugs or physical forms; AWD-12-281 from elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998]1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10b S*)-9-ethoxy-1,2,3,4,4a,10b-hexahydro-methoxy-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Suitable anticholinergic agents are those compounds that act as antagonists at the muscarinic receptor, in particular those compounds, which are antagonists of the M₁ and M₂ receptors. Exemplary compounds include the alkaloids of the belladonna plants as illustrated by the likes of atropine, scopolamine, homatropine, hyoscyamine; these compounds are normally administered as a salt, being tertiary amines.

Particularly suitable anticholinergics include ipratropium (e.g. as the bromide), sold under the name Atrovent, umeclidinium (e.g. as the bromide), glycopyrronium (e.g. as the bromide), oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO01/04118.

Suitable antihistamines (also referred to as H₁-receptor antagonists) include any one or more of the numerous antagonists known which inhibit H₁-receptors, and are safe for human use. All are reversible, competitive inhibitors of the interaction of histamine with H1-receptors. Examples include ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic the tertiary amine group with piperizine or piperidine. Exemplary antagonists are as follows: Ethanolamines: carbinoxamine maleate, clemastine fumarate, diphenylhydramine hydrochloride, and dimenhydrinate. Ethylenediamines: pyrilamine amleate, tripelennamine HCl, and tripelennamine citrate. Alkylamines: chlropheniramine and its salts such as the maleate salt, and acrivastine. Piperazines: hydroxyzine HCl, hydroxyzine pamoate, cyclizine HCl, cyclizine lactate, meclizine HCl, and cetirizine HCl. Piperidines: Astemizole, levocabastine HCl, loratadine or its descarboethoxy analogue, and terfenadine and fexofenadine hydrochloride or another pharmaceutically acceptable salt.

Azelastine hydrochloride is yet another Hireceptor antagonist which may be used in combination with a PDE4 inhibitor. Particularly suitable anti-histamines include methapyrilene and loratadine. In respect of combination products, co-formulation compatibility is generally determined on an experimental basis by known methods and may depend on chosen type of medicament dispenser action.

The medicament components of a combination product are suitably selected from the group consisting of anti-inflammatory agents (for example a corticosteroid or an NSAID), anticholinergic agents (for example, an M₁, M₂, M₁/M₂ or M₃ receptor antagonist), other β₂-adrenoreceptor agonists, anti-infective agents (e.g. an antibiotic or an antiviral), and antihistamines. All suitable combinations are envisaged.

Suitably, the co-formulation compatible components comprise a β₂-adrenoreceptor agonist and a corticosteroid; and the co-formulation incompatible component comprises a PDE-4 inhibitor, an anti-cholinergic or a mixture thereof. The β₂-adrenoreceptor agonists may for example be salbutamol (e.g., as the free base or the sulphate salt) or salmeterol (e.g., as the xinafoate salt) or formoterol (e.g., as the fumarate salt). The corticosteroid may for example, be a beclomethasone ester (e.g., the dipropionate) or a fluticasone ester (e.g., the propionate) or budesonide.

In one example, the co-formulation compatible components comprise fluticasone propionate and salmeterol, or a salt thereof (particularly the xinafoate salt) and the co-formulation incompatible component comprises a PDE-4 inhibitor, an anti-cholinergic (e.g. ipratropium bromide or tiotropium bromide) or a mixture thereof.

In another example, the co-formulation compatible components comprise budesonide and formoterol (e.g. as the fumarate salt) and the co-formulation incompatible component comprises a PDE-4 inhibitor, an anti-cholinergic (e.g. ipratropium bromide or tiotropium bromide) or a mixture thereof.

Medicament dispensers, as disclosed herein, have a variety of structural configurations and can be used for dispensing powders, tablets, capsules, pellets or pucks, or mixtures thereof for nasal, pulmonary or oral administration. In particular, the medicament dispensers can be used to dispense a powder comprising one or more active pharmaceutical ingredient(s)(API) and optionally, one or more micronized or non-micronized pharmaceutically acceptable carrier(s) and/or excipient(s), wherein the dispensing mechanism comprises of three steps to operate the medicament dispenser: Open, Dispense and Close.

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with one or more excipients or carriers which are suitable for inhalation. Suitable excipients or carriers include organic excipients such as polysaccharides (i.e. starch, cellulose and the like), lactose, glucose, mannitol, amino acids, magnesium stearate and maltodextrins, and inorganic excipients such as calcium carbonate or sodium chloride. In preferred embodiment, carrier or excipient is lactose.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The medicament dispensers described herein can be used for the treatment and prophylaxis of respiratory diseases, including but not limited to asthma, chronic obstructive pulmonary disease (COPD) bronchitis or chest infection.

The medicament dispenser can be a hand-held dispenser and can be operated by a single hand.

The medicament dispenser can be of circular form, non-circular form, an elongate form, an elliptical form, or other shapes.

The medicament dispenser can include a flat surface or a resting surface. In some embodiments, at least a portion of the medicament dispenser can be shaped for ease of grip by the user.

The medicament dispenser can be an inhalation device, in particular a dry powder inhaler (DPI).

The medicament dispenser can include a diskette assembly or a housing.

In some embodiments, the diskette assembly can include a base cover and a top cover for enclosing a medicament carrier, a medicament carrier advancing mechanism, inner housing assembly, dose indicator or dose counter and a lever. The top cover can lock with the base cover to form a flow channel (or opening). In some embodiments, the top cover can lock permanently with the base cover through welding techniques.

The flow channel can be in the form of a mouthpiece for oral use. In some embodiments, the flow channel can be in the form of a nozzle for insertion into the nasal cavity of a patient. In some embodiments, the flow channel can be in the form of exit channel if the medicament is in solid form such as tablets or capsules.

In some embodiments, the flow channel is a single outlet, which communicates with all of the distinct medicament on their release. Communication can be for example, via a common air channeling means (e.g. formed as an air-pipe or common manifold). In another embodiment, the flow channel and/or channeling device is shaped to encourage mixing of drug as a result of the airflow created by inhalation by the patient.

The term ‘mouthpiece’ is used to mean an element through which the patient may inhale. In one aspect, the inhalation is by oral means with the patient placing the mouthpiece in its mouth. In an alternative aspect, the mouthpiece comprises a nozzle for insertion into the nasal cavity of a patient.

In some embodiments, the mouthpiece can snap on the diskette assembly. The mouthpiece can further include a mouthpiece interface opening. The mouthpiece interface opening and flow channel can allow the distinct medicament portions to be dispensed to the user.

The diskette assembly can be of circular form, non-circular form, an elongate form or an ovular form. The diskette assembly can include a flat surface or a resting surface. In some embodiments, at least a portion of the diskette assembly can be shaped for ease of grip by the user.

The diskette assembly can include at least one medicament carrier carrying multiple distinct medicament portions.

In some embodiments, the diskette assembly can include a single or plural medicament carriers. The plural elongate form medicament carriers may be provided in any suitable configuration. One suitable configuration is the ‘side-by-side’ configuration, in which for example, two carriers (e.g. two coiled blister strips) are arranged to lie in sideways alignment with each other. Another suitable configuration is the ‘double-decker’ configuration, in which for example, two carriers (e.g. two coiled blister strips sharing the same coiling axis) are arranged to lay one on top of each other in the dispenser. In some embodiments, the plural medicament carriers can be a first medicament carrier and a second medicament carrier. The plural medicament carriers can be separated by a deck separator or deck separators. The deck separator can be used to allow the independent movement of the medicament carriers with no contact between medicament carriers and no friction between medicament carriers due to mutual contact. The deck separators can be locked in the inner housing assembly by locking heads.

In some embodiments, the multiple distinct dose portions can be provided to each carrier in uniform series. In particular, the spacing (i.e. pitch) between each dose portion is uniform throughout the series. In other aspects however, the spacing (i.e. pitch) may vary throughout the series (i.e. be non-uniform). In one aspect, the spacing (i.e. pitch) between each dose portion is equivalent for each carrier of the dispenser. That is to say, each medicament carrier is equivalently pitched. In other aspects, the spacing between each dose portion is non-equivalent for each carrier of the dispenser. Such variation of the spacing (i.e. pitch) from carrier to carrier can be used to enable flexibility in (combination) dosage patterns. In one particular example, the spacing (i.e. pitch) of a first carrier is arranged to be half that of a second carrier. This arrangement is beneficially employed where the dose interval (i.e. time between doses) of the medicament carried by the first carrier is twice that of the medicament carrier by the second carrier (e.g. in a twice daily/once daily type dosage regime).

In some embodiments, the medicament carrier carrying multiple distinct medicament portions can be in the form of a strip or tap. In some embodiments, the medicament carrier can be a reel of blisters containing multiple distinct blister pockets such as a blister pack or a blister strip. In some embodiments, the medicament carrier can be a blister pack in a laminate form. In some embodiments, access to the medicament(s) comprised within the blister pockets of reel of blisters is by any suitable access means including tearing, piercing or peeling apart the relevant blister pockets. The medicament carrier can be wound around a post of the inner housing assembly. One end of the medicament carrier can be in the form of a sealed loop that can be placed in a blister post of a winder ratchet. The other end of the medicament carrier can be placed in a slit of a wrapping wheel.

In some embodiments, the reel of blisters can be in the form of a peelable blister strip which includes blister pockets wherein each of the blister pockets has one dose of the medicament. In some embodiments, each blister pocket comprises or forms two or more separate chambers or compartments, for example two compartments. The compartments are formed by a partition wall dividing the blister pocket. In some embodiments, the peelable blister strip comprises a base sheet in which blisters are formed to define pockets therein for containing distinct medicament dose portions and a lid sheet or lamination which is hermetically sealed to the base sheet except in the region of the blisters in such a manner that the lid sheet and the base sheet can be peeled apart. The blister pocket having compartments may be covered by common lid sheet. The lamination of the blister pockets is peeled via a constant tension torque applied by the winding assembly.

In some embodiments, the diskette assembly can include at least one piercing element for puncturing the lamination or lid sheet of the blister pocket. In some embodiments, the diskette assembly comprises at least one piercing elements for puncturing the lamination or lid sheet of the compartments of one blister pocket simultaneously, for example two piercing elements.

In some embodiments, the blister pocket from each of the reel of blisters can include medicament comprising one or more active pharmaceutical ingredient(s). In some embodiments, the blister pocket can include medicament comprising a single active pharmaceutical ingredient. In some embodiments, the blister pocket can include medicament comprising two or more active pharmaceutical ingredients. For example, the blister pocket can include medicament comprising two or three active pharmaceutical ingredients.

In some embodiments, the diskette assembly can include a lever which comprises an arcuate wall with a lever surface, a center and an arm which extends inwardly from the wall and carries an arcuate array of teeth at its distal end (i.e. lever teeth).

In some embodiments, the diskette assembly can include a lever which comprises an arcuate wall with a driver, a center and an arm which extends inwardly from the wall and carries an arcuate array of teeth at its distal end (i.e. lever teeth).

In some embodiments, the diskette assembly can include a lever which comprises an arcuate wall with a bar, a center and an arm which extends inwardly from the wall and carries an arcuate array of teeth at its distal end (i.e. lever teeth).

In some embodiments, the lever can include a lever flap that uncovers and covers the mesh of the inner housing assembly during actuation and release.

In some embodiments, the diskette assembly can include a central gear. In further embodiment, the central gear can include a boss, a bearing and teeth.

In some embodiments, the diskette assembly can include an idle gear. In further embodiment, the idle gear comprises teeth.

The teeth of the central gear fix with the teeth of the idle gear to actuate the medicament carrier advancing mechanism.

In some embodiments, the inner housing assembly can house the medicament carrier. The inner housing assembly can include a mesh or meshes that allow de-agglomeration of the medicament. During dosing operation, the cuvette on the blister cuvette is aligned with the mesh which can allow the medicament to be dispensed through the flow channel and mouthpiece interface opening to the user.

In some embodiments, the medicament dispenser comprises an outer cover. The outer cover can be mounted to the diskette assembly by a suitable mounting such as a pivot mounting wherein the outer cover pivots around the diskette assembly. In some embodiments, the outer cover can pivots about studs of the diskette assembly via the mounting holes.

In some embodiments, the medicament dispenser further comprises an inner cover. The outer cover and the inner cover can be mounted to the diskette assembly by a suitable mounting such as a pivot mounting wherein the outer cover and the inner cover pivot around the body of the diskette assembly. In another embodiment, the inner cover acts as hinge to the outer cover. The inner cover also pivots about studs on the diskette assembly.

In some embodiments, the outer cover can be adapted to couple with the lever and the medicament carrier advancing mechanism.

In some embodiments, the outer cover can be adapted to couple with the inner cover and the medicament carrier advancing mechanism via the lever.

In some embodiments the outer cover can be adapted to couple with the lever and the medicament carrier advancing mechanism via the inner cover.

In some embodiments, the outer cover can be adapted to couple with the central gear and the medicament carrier advancing mechanism via the idle gear.

In some embodiments, the outer cover can be movably mounted to the medicament dispenser for sequential movement from an initial position, in which the flow channel is covered, to a dispense position, in which the flow channel is uncovered. In some embodiments, the flow channel is fully revealed when the outer cover is in a dispense position.

In some embodiments, the movement of the outer cover from the initial position rotates the lever and actuate the medicament carrier advancing mechanism. For example, any rotation of the outer cover can cause a corresponding rotation of the lever.

In some embodiments, the movement of the outer cover from the initial position rotates the inner cover and the lever and actuate the medicament carrier advancing mechanism. For example, any rotation of the outer cover can cause a corresponding rotation of the inner cover and lever.

In some embodiments, the movement of the outer cover from the initial position can rotate the central gear and the idle gear to actuate the medicament carrier advancing mechanism.

In some embodiments, the movement of the outer cover from the initial position results in actuation of the medicament carrier advancing mechanism.

In some embodiments, the outer cover can include at least two ridges. In particular a first ridge and a second ridge.

The first ridge can rotate the lever from the initial position of the outer cover to the dispense position of the outer cover to actuate the medicament carrier advancing mechanism.

The second ridge can retract the lever back from the dispense position of the outer cover to the initial position of the outer cover.

In some embodiments, the outer cover can include a plunger. In some embodiments, the plunger having a plunger nose; a plunger cover; and compression spring.

The plunger can rotate the lever from the initial position of the outer cover to the dispense position of the outer cover to actuate the medicament carrier advancing mechanism.

The plunger can retract the lever back from the dispense position of the outer cover to the initial position of the outer cover

The compression spring can push the plunger to engage the plunger nose with the driver.

In some embodiments, the outer cover can at include at least two ridges. In particular a first ridge and a second ridge.

In some embodiments, the first ridge can rotate the inner cover thereby moving the lever from the initial position of the outer cover to the dispense position of the outer cover to actuate the medicament carrier advancing mechanism.

In some embodiments, the second ridge can retract the inner cover thereby moving the lever back from the dispense position of the outer cover to the initial position of the outer cover.

In some embodiments, the inner cover can include a protrusion and a groove.

In some embodiments, the second ridge of the outer cover can engage the protrusion of the inner cover when outer cover moves from the dispensed position to the initial position. In some embodiments, the groove of the inner cover can engage with the bar.

In some embodiments, the outer cover comprises a plunger.

In some embodiments, the plunger can rotate the inner cover thereby moving the lever from the initial position of the outer cover to the dispense position of the outer cover to actuate the medicament carrier advancing mechanism.

In some embodiments, the plunger can retract the inner cover thereby moving the lever back from the dispense position of the outer cover to the initial position of the outer cover.

In some embodiments, the dispensing of the medicament provides continuous movement of the outer cover from the initial position to the dispense position of the outer cover without any intermediate position.

In some embodiments, the dispensing of the medicament provides continuous movement of the outer cover from the initial position to rotate the lever and actuate the medicament carrier advancing mechanism.

In some embodiments, the dispensing of the medicament provides continuous movement of the outer cover from the initial position to rotate the inner cover and the lever and actuate the medicament carrier advancing mechanism.

In some embodiments, the dispensing of the medicament can provide continuous movement of the outer cover from the initial position results in actuation of the medicament carrier advancing mechanism

In some embodiments, the dispensing of the medicament can provide continuous movement of the outer cover from the initial position to rotate the central gear and the idle gear to actuate the medicament carrier advancing mechanism.

The term “initial position” as used herein is interchangeable with “Initial state”, “rest position”, “rest state”, “home position” or “closed position” and refers to the position of the outer cover wherein the outer cover covers the flow channel and/or mouthpiece, and user cannot access the medicament.

The term “dispense position” as used herein is interchangeable with “dispense state” “open position”, “open state”, “inhaled position” and refers to the position of the outer cover wherein the outer cover does not cover the flow channel and/or mouthpiece, and user can access the medicament.

In some embodiments, the dispensing of the medicament does not provide any idle movement of the outer cover.

In some embodiments, the outer cover is of circular form, non-circular form, an elongate form, an elliptical form, or any other shape. In some embodiments, the outer cover provides a flat surface or a resting surface.

In some embodiments, at least a portion of the outer cover is shaped for ease of grip by the user.

In some embodiments, the medicament carrier advancing mechanism for actuating the distinct medicament portions carried by the medicament carrier can include a cuvette ratchet, a blister cuvette, a winding assembly, an interim gear and a wrapping wheel. The blister cuvette can include a driver surface, cuvettes, gears and grooves. The blister cuvette can be driven by the ratcheting mechanism of the cuvette ratchet. The cuvettes on the blister cuvette can locate the individual blister pocket on the medicament carriers. The gears of the blister cuvette can mate with the interim gear and the winder gear. The grooves on the blister cuvette can mate with a spring on the base cover for providing an auditory feedback to the user to intake the medicament. The cuvette ratchet can include a bearing, ratchets and spur gears. The cuvette ratchet can ride over the anterior boss of the base cover. The cuvette ratchet can be guided on the anterior boss by a bearing. The cuvette ratchet can be driven by the lever via the spur gears. The ratchets located diametrically opposite on the cuvette ratchet drive the blister cuvette via the driver surface of the blister cuvette. The ratcheting mechanism of the cuvette ratchet prevents the rotation of the blister cuvette in the opposite direction. The ratchets ride over the inner wall of the blister cuvette without rotating the blister cuvette. The winder rotor can include a winding surface, a recess opening and a winding post. In some embodiments, the winder rotor can include a ridge for separating the laminations of the plural medicament carriers during the winding process. The winder ratchet can include a ratchet, locking posts, a blister post and a flange. The winder gear can include internal gears and external gears. The wrapping wheel can include a slit, gears and distal gears.

In some embodiments, the base cover can hold and encase the medicament carrier advancing mechanism. The base cover can include a winder boss, an anterior boss, a stud, a platform, a curved ridge, circumferential ribs, a spring, a locking surface and a window.

In some embodiments, the top cover can include a hollow boss for supporting the wrapping wheel, a post for supporting the cuvette ratchet, a vent for allowing passage for air into the dispenser during dosing maneuver.

In some embodiments, the actuation of the medicament carrier advancing mechanism rotates the cuvette ratchet and the blister cuvette synchronously and thereby advances the medicament carrier to align with the flow channel for dispensing the medicament.

In some embodiment, the medicament dispenser comprises a dose counting or an indicating mechanism for counting or indicating number of doses released from the dispenser.

In some embodiments, the medicament dispenser comprises an actuation or dose counter for counting the number of actuations or releases of dose from the medicament dispenser. The dose counter may count the number of doses left to be taken or the number of doses taken. The dose counter may be mechanical (e.g., non-electronic) or non-mechanical (e.g., electronic, digital) in form.

In some embodiments, the medicament dispenser comprises a mechanism of actuation wherein the mechanism of actuation also provides for a mechanism for increase in dose counter reading during dispensation of the medicament.

In some embodiments, the medicament dispenser comprises a mechanism of actuation wherein the movement of the mechanism of actuation also provides for a mechanism for increase in a mechanical (i.e. non-electronic) and a non-mechanical (e.g., electronic, digital) dose counter reading during dispensation of the medicament.

In some embodiments, the dose counter can be a part of diskette assembly such as a mechanical dose ring. The dose counter can be located in a compartment of the outer cover having dedicated space to accommodate said dose counter.

In some embodiments, the medicament dispenser comprises a space for a power source (e.g., battery), electronic circuit and electronic measurement instruments. The power source (e.g., battery), electronic circuit and electronic measurement instruments can be located in a compartment of the outer cover having dedicated space to accommodate said power source (e.g., battery), electronic circuit and electronic measurement instruments.

In some embodiments, the medicament dispenser comprises an electronic data management system. The electronic data management system can include input/output capability and comprises a memory for storage of data; a microprocessor for performing operations on said data; and a transmitter for transmitting a signal relating to the data or the outcome of an operation on the data. The electronic data management system may be integral with the body of the dispenser. Alternatively, the electronic data management system forms part of a base unit that is reversibly associable with the body.

In some embodiments, the medicament dispenser can be made from the material of constructions comprises of acrylonitrile butadiene styrene (ABS), polycarbonate/acrylonitrile butadiene styrene terpolymer blend (PC/ABS), Polyoxymethylene (POM), nylon and silicone rubber.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein said outer cover being movably mounted to the dispenser for sequential movement from an initial position, in which said flow channel is covered, to a dispense position, in which said flow channel is uncovered.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein said outer cover is adapted to couple with said medicament carrier advancing mechanism such that the movement of the outer cover from an initial position rotates the lever to actuate the medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein a first ridge of the outer cover is rotated along with the lever from an initial position to a dispense position to actuate the medicament carrier advancing mechanism for dispensing the distinct medicament portions.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein a second ridge of the outer cover retracts the lever through rotation of the outer cover in reverse direction from a dispense position to an initial position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein the mechanism of actuation provides continuous movement of outer cover without any idling position between an initial position and a dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein mechanism of actuation rotates a cuvette ratchet by discrete angular movement through spur gears.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein a cuvette rotates synchronously with a cuvette ratchet in one direction, thereby advancing the medicament carrier.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; and b) an outer cover for said flow channel comprises at least two ridges, wherein the outer cover couples directly with medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the flow channel is covered by the outer cover in an initial position, and fully revealed when the outer cover is in a dispense position.

In some embodiments, a medicament dispenser for dispensing medicament comprises: a) a diskette assembly comprising: i) at least one medicament carrier carrying multiple distinct medicament portions; ii) mechanism of actuation for dispensing the distinct medicament portions; iii) a flow channel; iv) a mouthpiece; v) a lever; and b) an outer cover for said mouthpiece comprises at least two ridges, wherein the mouthpiece arranged such that the flow channel is covered by the outer cover in its initial position, and fully revealed when the outer cover is in its dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a mouthpiece; v) a lever; and b) an outer cover for said mouthpiece comprises at least two ridges, wherein the mechanism of actuation provides continuous movement of the outer cover such that there is no intermediate position for the outer cover between an initial position and a dispense position.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein the coupling of the outer cover with the medicament carrier advancing mechanism is adapted such the movement of the cover from an initial position to a dispense position results in actuation of dispensing mechanism without any idle movement of the outer cover.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein said dispenser is an inhalation device.

In some embodiments, a medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges, wherein the dispenser is suitable for dispensing medicament for the treatment of asthma, chronic obstructive pulmonary disease (COPD), bronchitis or chest infection.

In some embodiments, a medicament dispenser for dispensing medicament comprises: a) a diskette assembly comprising: a medicament carrier; a flow channel; a medicament carrier advancing mechanism; and a lever; and b) an outer cover comprising a first ridge; and a second ridge, wherein the outer cover is movable form an at initial position, in which the outer cover covers said flow channel, to a dispense position to uncover the flow channel, and the movement of the outer cover coupled to said lever and said medicament carrier advancing mechanism by the first ridge and the second ridge that are arranged such that movement of the outer cover from the initial position to the dispense position results in rotation of the lever and actuation of the medicament carrier advancing mechanism by the first ridge of the outer cover thereby advances the medicament carrier to align that with the flow channel and through which a user can access the medicament from said flow channel, and movement of the outer cover from the dispensed position to the initial position results in retraction of the lever by the second ridge of the outer cover back to the initial position.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the outer cover is adapted to couple with the lever and the medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein said outer cover being movably mounted to the dispenser for sequential movement from an initial position, in which said flow channel is covered, to a dispense position, in which said flow channel is uncovered.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein said outer cover is adapted to couple with said medicament carrier advancing mechanism such that the movement of the outer cover from an initial position rotates the lever to actuate the medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein the plunger of the outer cover is rotated along with the lever from an initial position to a dispense position to actuate the medicament carrier advancing mechanism for dispensing the distinct medicament portions.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein the plunger of the outer cover retracts the lever through rotation of the outer cover in reverse direction from a dispense position to an initial position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever and b) an outer cover for said flow channel comprises a plunger, wherein the mechanism of actuation provides continuous movement of outer cover without any idling position between an initial position and a dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever and b) an outer cover for said flow channel comprises a plunger, wherein mechanism of actuation rotates a cuvette ratchet by discrete angular movement through spur gears.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever and b) an outer cover for said flow channel comprises a plunger, wherein a cuvette rotates synchronously with a cuvette ratchet in one direction, thereby advancing the medicament carrier.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever and b) an outer cover for said flow channel comprises a plunger, wherein the outer cover couples directly with medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises a plunger, wherein the flow channel is covered by the outer cover in an initial position, and fully revealed when the outer cover is in a dispense position.

In some embodiments, a medicament dispenser for dispensing medicament comprises: a) a diskette assembly comprising: i) at least one medicament carrier carrying multiple distinct medicament portions; ii) mechanism of actuation for dispensing the distinct medicament portions; iii) a flow channel; iv) a mouthpiece; v) a lever and b) an outer cover for said mouthpiece comprises a plunger, wherein the mouthpiece arranged such that the flow channel is covered by the outer cover in its initial position, and fully revealed when the outer cover is in its dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a mouthpiece; v) a lever and b) an outer cover for said mouthpiece comprises a plunger, wherein the mechanism of actuation provides continuous movement of the outer cover such that there is no intermediate position for the outer cover between an initial position and a dispense position.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein the coupling of the outer cover with the medicament carrier advancing mechanism is adapted such the movement of the cover from an initial position to a dispense position results in actuation of dispensing mechanism without any idle movement of the outer cover.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein said dispenser is an inhalation device.

In some embodiments, a medicament dispenser for dispensing a medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever and b) an outer cover for said flow channel comprises a plunger, wherein the dispenser is suitable for dispensing medicament for the treatment of asthma, chronic obstructive pulmonary disease (COPD), bronchitis or chest infection.

In some embodiments, a medicament dispenser for dispensing medicament comprises: a) a diskette assembly comprising: a medicament carrier; a flow channel; a medicament carrier advancing mechanism; and a lever having a driver; and b) an outer cover comprising a plunger having a plunger nose; a plunger cover; and compression spring, wherein the outer cover is movable from an initial position, in which the outer cover covers said flow channel, to a dispense position to uncover the flow channel, and the movement of the outer cover coupled to the driver and the medicament carrier advancing mechanism by the plunger nose that are arranged such that movement of the outer cover from the initial position to the dispense position results in rotation of the lever and actuation of the medicament carrier advancing mechanism by the plunger nose of the outer cover thereby advances the medicament carrier to align that with the flow channel and through which a user can access the medicament from said flow channel, and movement of the outer cover from the dispensed position to the initial position results in retraction of the lever by the plunger nose of the outer cover back to the initial position.

In some embodiments, a medicament dispenser for dispensing medicament comprises: a) a diskette assembly comprising: i) at least one medicament carrier carrying multiple distinct medicament portions; ii) a medicament carrier advancing for actuating the distinct medicament portions; iii) a flow channel; iv) a lever; b) an outer cover for said flow channel comprises at least two ridges; and c) an inner cover.

In some embodiments, medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein said outer cover being movably mounted to the dispenser for sequential movement from an initial position, in which said flow channel is covered, to a dispense position, in which said flow channel is uncovered.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein said outer cover is adapted to couple with said medicament carrier advancing mechanism such that the movement of the outer cover from an initial position rotates the lever to actuate the medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein a first ridge of the outer cover is rotated along with the lever from an initial position to a dispense position to actuate the medicament carrier advancing mechanism for dispensing the distinct medicament portions.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein a second ridge of the outer cover retracts the lever through rotation of the outer cover in reverse direction from a dispense position to an initial position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges, and c) an inner cover, wherein the mechanism of actuation provides continuous movement of outer cover without any idling position between an initial position and a dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges, and c) an inner cover, wherein mechanism of actuation rotates a cuvette ratchet by discrete angular movement through spur gears.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges; and c) an inner cover, wherein a cuvette rotates synchronously with a cuvette ratchet in one direction, thereby advancing the medicament carrier.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a lever; b) an outer cover for said flow channel comprises at least two ridges; and c) an inner cover, wherein the outer cover couples directly with medicament carrier advancing mechanism.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel; and c) an inner cover, wherein the flow channel is covered by the outer cover in an initial position, and fully revealed when the outer cover is in a dispense position.

In some embodiments, a medicament dispenser for dispensing medicament comprises: a) a diskette assembly comprising: i) at least one medicament carrier carrying multiple distinct medicament portions; ii) mechanism of actuation for dispensing the distinct medicament portions; iii) a flow channel; iv) a mouthpiece; v) a lever; b) an outer cover for said mouthpiece comprises at least two ridges; and c) an inner cover, wherein the mouthpiece arranged such that the flow channel is covered by the outer cover in its initial position, and fully revealed when the outer cover is in its dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a mouthpiece; v) a lever; b) an outer cover for said mouthpiece comprises at least two ridges; and c) an inner cover, wherein the mechanism of actuation provides continuous movement of the outer cover such that there is no intermediate position for the outer cover between an initial position and a dispense position.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein the coupling of the outer cover with the medicament carrier advancing mechanism is adapted such the movement of the cover from an initial position to a dispense position results in actuation of dispensing mechanism without any idle movement of the outer cover.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel comprises at least two ridges; and c) an inner cover, wherein said dispenser is an inhalation device.

In some embodiments, a medicament dispenser for dispensing a medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a lever; b) an outer cover for said flow channel comprises at least two ridges; and c) an inner cover, wherein the dispenser is suitable for dispensing medicament for the treatment of asthma, chronic obstructive pulmonary disease (COPD), bronchitis or chest infection.

In some embodiments, a medicament dispenser for dispensing medicament comprises: a) a diskette assembly comprising: a medicament carrier; a flow channel; a medicament carrier advancing mechanism; and a lever having a bar; b) an outer cover comprising a first ridge; and a second ridge; and c) an inner cover comprising a protrusion and a groove; wherein the outer cover is movable form an at initial position, in which the outer cover covers said flow channel, to a dispense position to uncover the flow channel, and the movement of the outer cover coupled to the inner cover and the medicament carrier advancing mechanism via said bar by the first ridge and the second ridge that are arranged such that movement of the outer cover from the initial position to the dispense position results in rotation of the inner cover and the lever and actuation of the medicament carrier advancing mechanism by the first ridge of the outer cover thereby advances the medicament carrier to align that with the flow channel and through which a user can access the medicament from said flow channel, and movement of the outer cover from the dispensed position to the initial position results in retraction of the inner cover thereby the lever by the second ridge of the outer cover back to the initial position.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel comprises, wherein said outer cover being movably mounted to the dispenser for sequential movement from an initial position, in which said flow channel is covered, to a dispense position, in which said flow channel is uncovered.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein said outer cover is adapted to couple with said medicament carrier advancing mechanism such that the movement of the outer cover from an initial position rotates the central gear and the idle gear to actuate the medicament carrier advancing mechanism.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a central gear; v) an idle gear; and b) an outer cover for said flow, wherein the mechanism of actuation provides continuous movement of outer cover without any idling position between an initial position and a dispense position.

In some embodiments, a medicament dispenser for dispensing medicament comprising: a) a diskette assembly comprises: i) at least one medicament carrier carrying multiple distinct medicament portions; ii) a mechanism of actuation for dispensing the distinct medicament portions; iii) a flow channel; iv) a central gear; v) an idle gear; and b) an outer cover for said flow channel, wherein a cuvette rotates synchronously with a cuvette ratchet in one direction, thereby advancing the medicament carrier.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a central gear; v) an idle gear; and b) an outer cover for said flow channel, wherein the outer cover is adapted to couple with the central gear and the medicament carrier advancing mechanism via said idle gear.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the flow channel is covered by the outer cover in an initial position, and fully revealed when the outer cover is in a dispense position.

In some embodiments, a medicament dispenser for dispensing medicament comprises: a) a diskette assembly comprising: i) at least one medicament carrier carrying multiple distinct medicament portions; ii) mechanism of actuation for dispensing the distinct medicament portions; iii) a flow channel; iv) a mouthpiece; iv) a central gear; v) an idle gear; and b) an outer cover for said mouthpiece, wherein the mouthpiece arranged such that the flow channel is covered by the outer cover in its initial position, and fully revealed when the outer cover is in its dispense position.

In some embodiments, a medicament dispenser for dispensing medicament comprises: a) a diskette assembly comprising: i) at least one medicament carrier carrying multiple distinct medicament portions; ii) a mechanism of actuation for dispensing the distinct medicament portions; iii) a flow channel; iv) a mouthpiece iv) a central gear; v) an idle gear; and b) an outer cover for said mouthpiece comprises, wherein the mechanism of actuation provides continuous movement of the outer cover such that there is no intermediate position for outer cover between an initial position and a dispense position.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the coupling of the outer cover with the medicament carrier advancing mechanism is adapted such the movement of the cover from an initial position to a dispense position results in actuation of dispensing mechanism without any idle movement of the outer cover.

In some embodiments, a medicament dispenser for dispensing medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the dispenser is an inhalation device.

In some embodiments, a medicament dispenser for dispensing a medicament comprises: a) a diskette assembly comprising: i) at least one medicament carrier carrying multiple distinct medicament portions; ii) a medicament carrier advancing mechanism for actuating the distinct medicament portions; iii) a flow channel; iv) a central gear; v) an idle gear; and b) an outer cover for said flow channel, wherein the dispenser is suitable for dispensing medicaments for the treatment of asthma, chronic obstructive pulmonary disease (COPD), bronchitis or chest infection.

In some embodiments, a medicament dispenser for dispensing medicament comprises: a) a diskette assembly comprising: a medicament carrier; a flow channel; a medicament carrier advancing mechanism; a central gear; and an idle gear; and b) an outer cover, wherein the outer cover is movable form an at initial position, in which the outer cover covers said flow channel, to a dispense position to uncover the flow channel, and the movement of the outer cover coupled to said medicament carrier advancing mechanism by the central gear and the idle gear that are arranged such that movement of the outer cover from the initial position to the dispense position results in actuation of the medicament carrier advancing mechanism by the central gear and the idle gear thereby advances the medicament carrier to align that with the flow channel, and through which a user can access the medicament from said flow channel, and movement of the outer cover from the dispense position to the initial position results in rotation of the central gear and the idle gear without advancing the medicament carrier.

It is intended that the scope of the present invention herein disclosed should not be limited by any particular embodiment described herein. While various embodiments of the present invention have been described above, it should be noted that they have been presented by way of example only, and not limitation. Numerous changes to the disclosed embodiments can be made in accordance with the disclosure herein without departing from the spirit or scope of the invention.

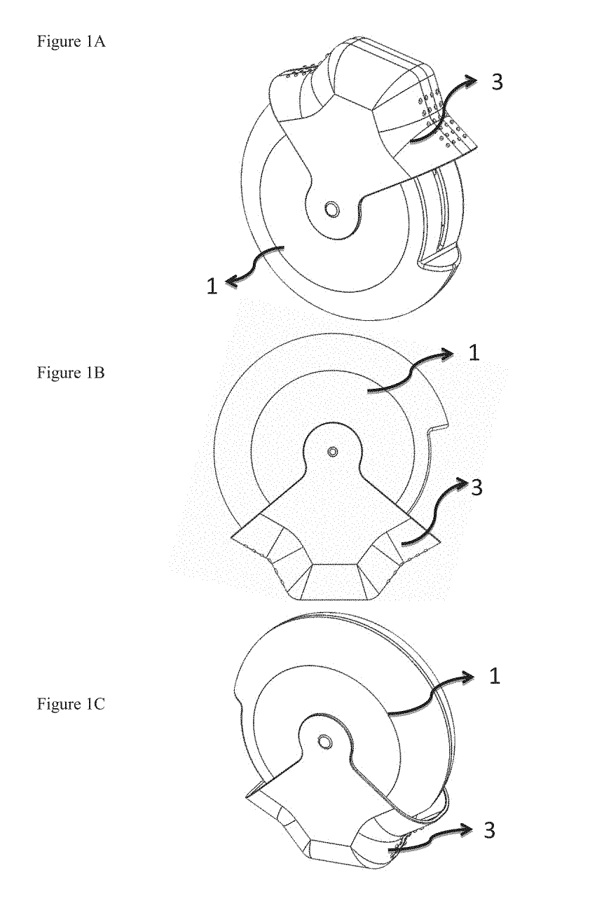

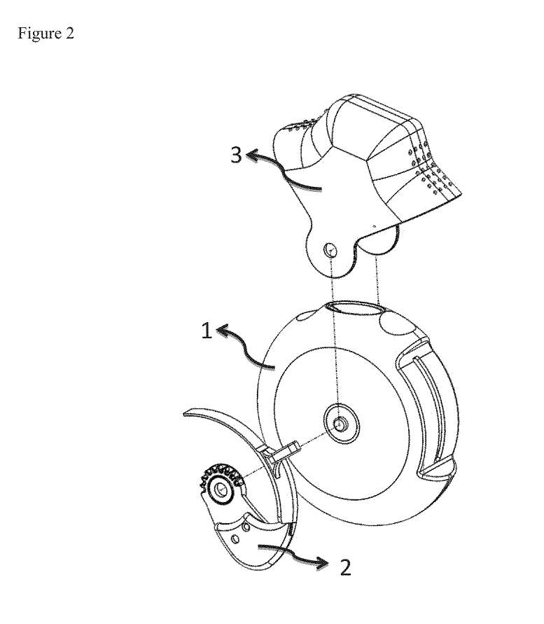

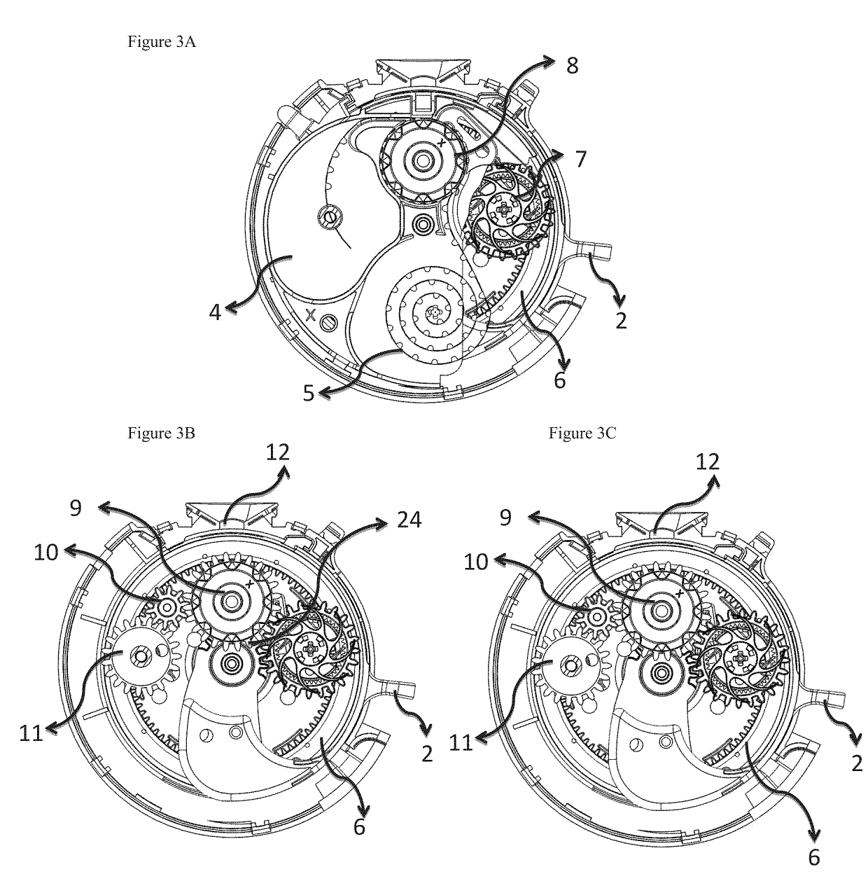

FIGS. 1A, 1B and 1C illustrate different views of the medicament dispenser according to one aspect of the disclosure. The medicament dispenser can include a diskette assembly (1) which houses a reel of blisters (not shown) and the blister advancing mechanism. The blister advancing mechanism (see FIGS. 2 and 3A-3C) is operated (or actuated) via the lever (2). The lever (2) can be operated (or actuated) via the outer cover (3).

As shown in FIGS. 3A, 3B, and 3C, the diskette assembly (1) can house a reel of blisters (5) and the blister advancing mechanism. The reel of blisters (5) can incorporate individual blister pockets having a powder, tablet, capsule, pellet or puck to be delivered by the medicament dispenser. The blister advancing mechanism is operated (or actuated) via the lever (2). The lever (2) can be operated (or actuated) via the outer cover (3). The operation (or actuation) of lever (2) can rotate the cuvette ratchet (9) by a discrete angular movement via the spur gears (24). The blister cuvette (8) can rotate synchronously and unidirectional with the cuvette ratchet (9), thereby advancing the blister pockets from the reel of blisters (5). Any movement by the lever (2) can lead to corresponding movement of the cuvette ratchet (9). The reel of blisters (5) that incorporates the blister pockets can be laminated at other end.

During operation of the medicament dispenser, the blister pockets align with the flow channel (12) of the diskette assembly. The lamination of the blister pockets can be peeled during each blister movement via a constant tension torque applied by the winding assembly (7).

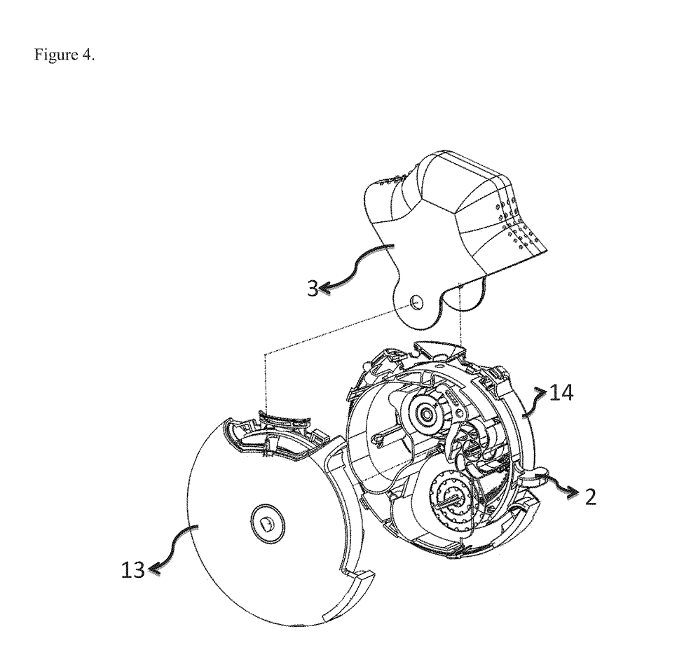

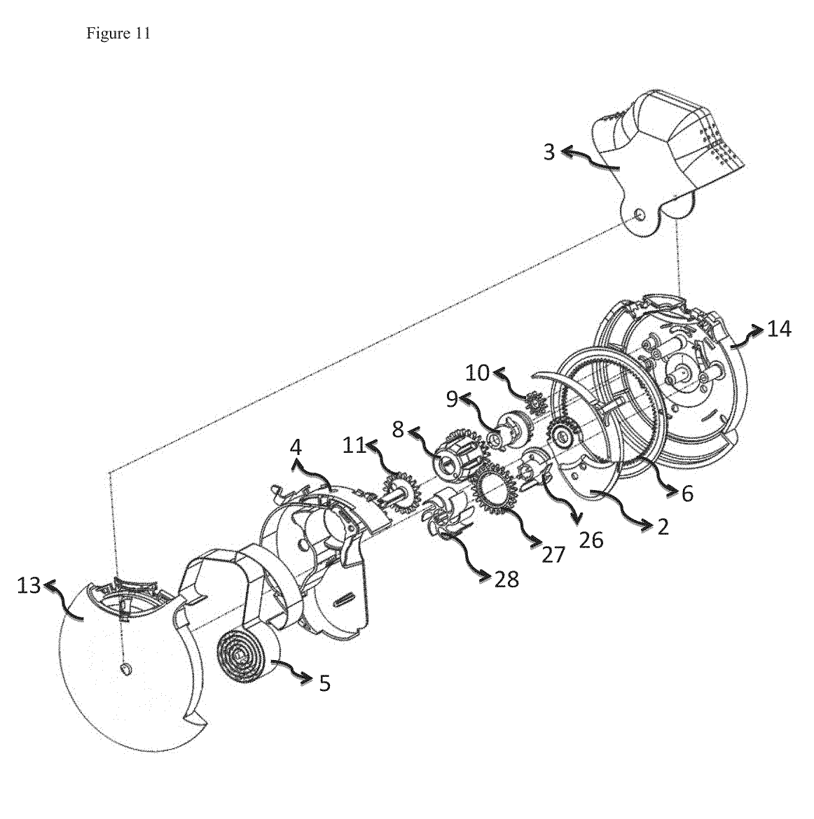

During operation of the medicament dispenser, the lamination winds around the winding assembly (7) (see FIG. 3A). The winding assembly (7) can include the winder ratchet (26), winder gear (27) and the winder rotor (28) (see FIG. 11). By the same mechanism the blister pockets are advanced via the wrapping wheel (11) (see FIGS. 3B and 3C). The wrapping wheel (11) rotates in the opposite direction of the blister cuvette (8) via the interim gear (10). The reel of blisters can be separated from the blister advancing assembly via an inner housing assembly (4) (see FIG. 3A). The blister advancing mechanism can be protected by a top cover (13) (FIG. 4). The top cover (13) locks with the base cover (14). The outer cover (3) pivots on the diskette assembly (1) which provides for the final assembly of the medicament dispenser.

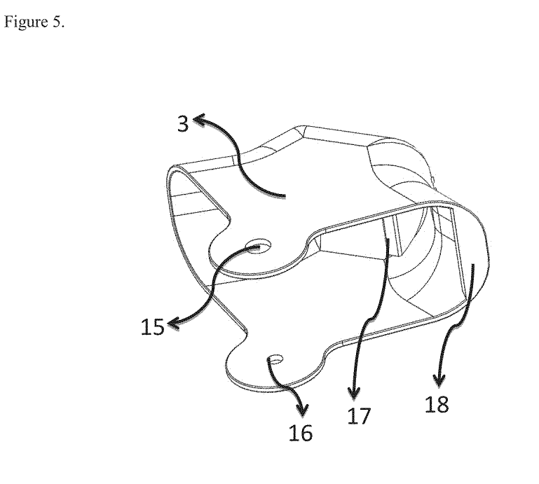

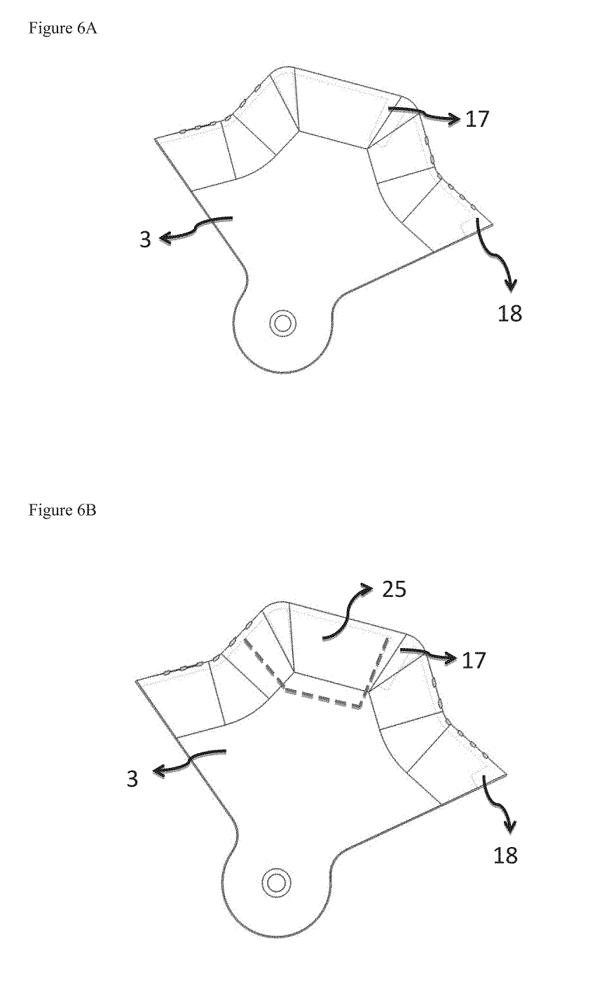

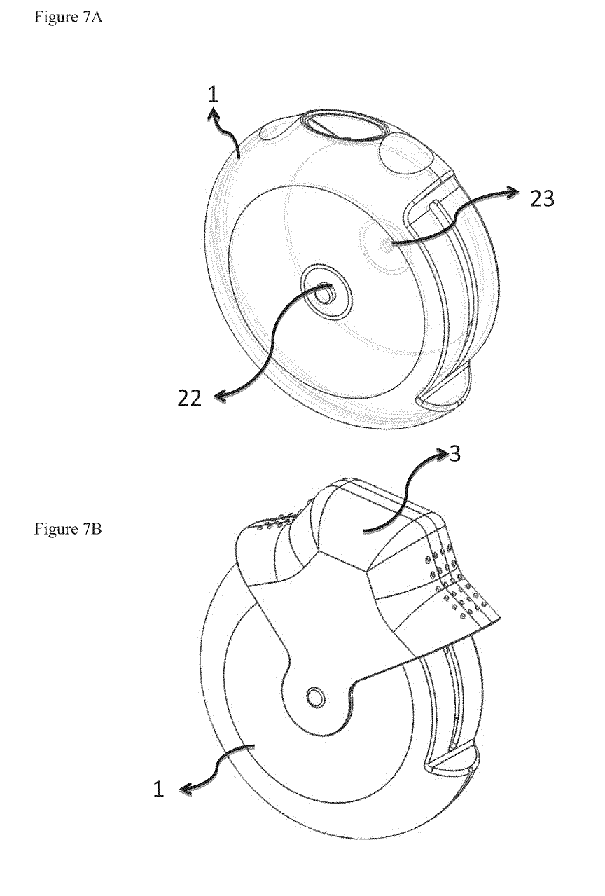

The outer cover (3) (FIGS. 5 and 6A-6B) can include mounting holes (15) and (16) that lock with respective studs (22) and (23) on the diskette assembly (1) (see FIGS. 7A-7B). The studs (22) and (23) on the diskette assembly (1) provide a hinge about which the outer cover (3) rotates. The studs 22 and 23 provide a hinge for the outer cover (3) which engages with the lever (2) to actuate the blister advancing mechanism inside the diskette assembly (1). The first ridge (17) is an actuation ridge on the outer cover (3). The second ridge (18) retracts the lever (2) back to initial state. In some embodiments of the outer cover (3) (see FIGS. 6A and 6B) the compartment (25) is a dedicated space to accommodate for a power source and electronic instruments.

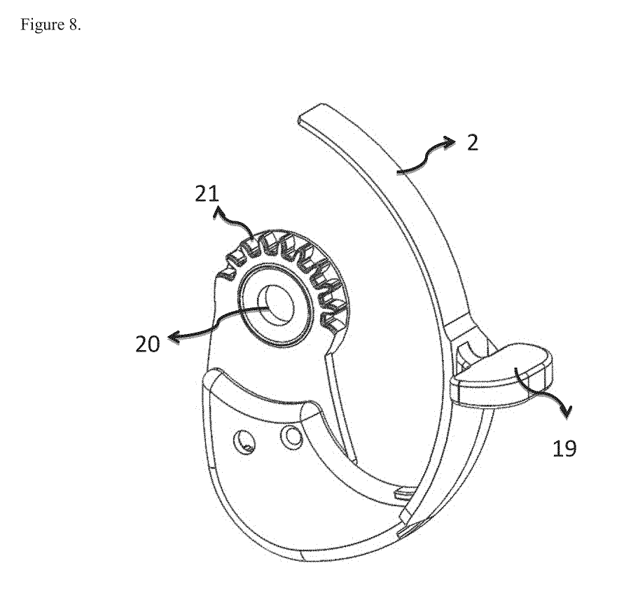

The lever (2) (FIG. 8) can include a lever surface (19) that provides a contact surface for the first ridge (17) on the outer cover (3). The lever (2) can include a center (20). As the lever (2) rotates nominally (for example, to an angle of 20°), the blister advancing mechanism inside the diskette assembly (1) can be rotated via the lever teeth (21).

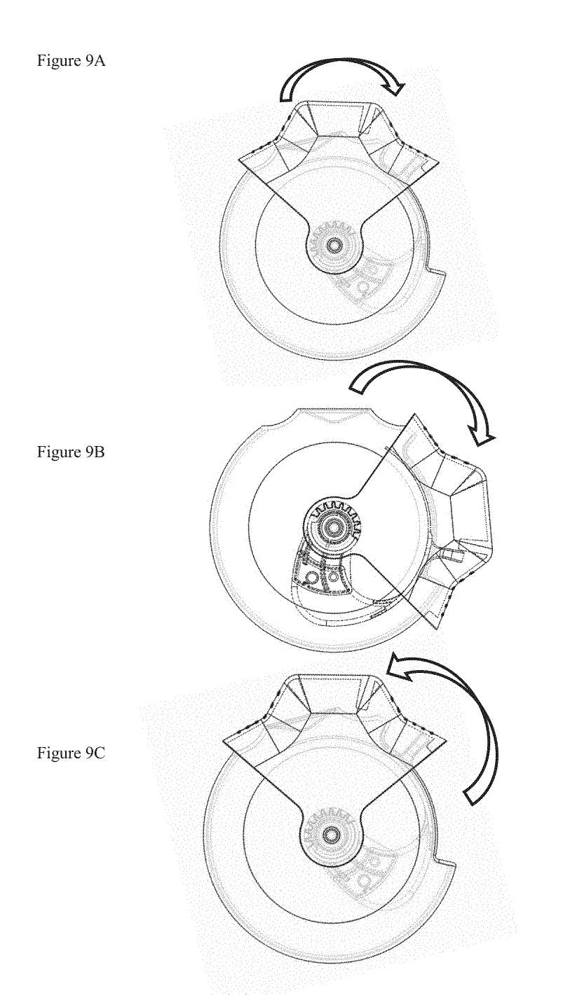

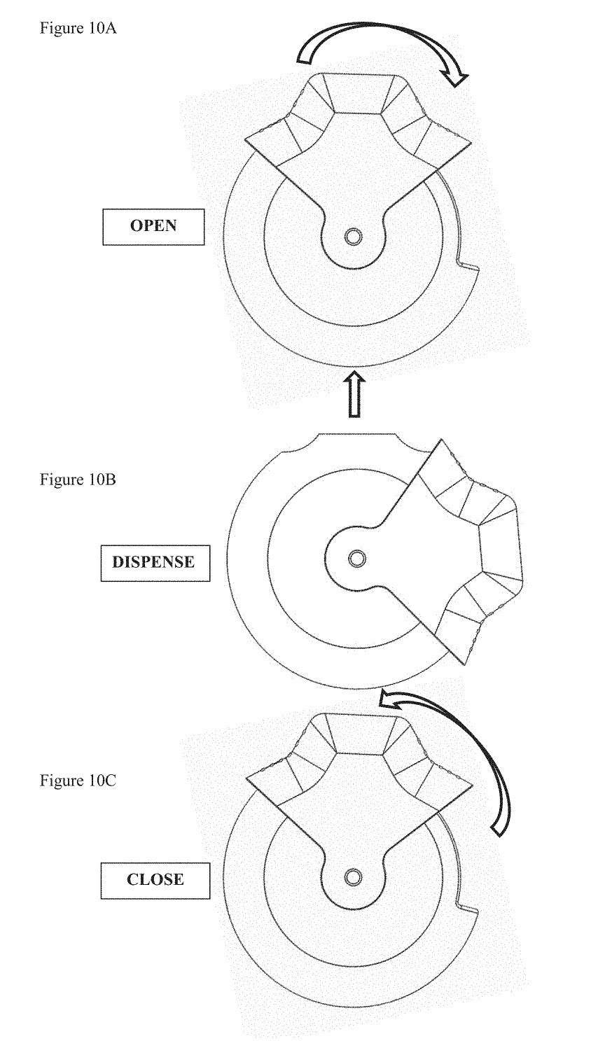

The mechanism of operation of the medicament dispenser is presented in FIGS. 9A-9C and 10A-10C. The inhaler assembly can include a diskette assembly, a lever and an outer cover. The blister advancing mechanism is located within the diskette assembly. In the initial (rest) state the outer cover (3) covers the flow channel (12) of the diskette assembly (1). When the outer cover (3) of the medicament dispenser is rotated clockwise (opened) the first ridge (17) on the outer cover comes in contact with the lever (2) on the lever surface (19). The lever (2) is in a rest/initial state before outer cover is operated (FIG. 9A). As the outer cover (3) is rotated, the lever (2) rotates along with the first ridge (17) and actuates the blister advancing mechanism inside the diskette assembly (1) (FIG. 9B). The actuation of lever rotates the cuvette ratchet (9) by a discrete angular movement via the spur gears (24). The blister cuvette (8) rotates synchronously with the cuvette ratchet (9) uni-directionally, thereby advancing the blister from the reel of blisters (5). Any movement of the outer cover (3) and/or lever (2) can lead to corresponding movement of the blister cuvette (8) and/or cuvette ratchet (9). At this stage the blister pockets align with the flow channel (12) of the diskette assembly. The lamination of the blister pockets can be peeled via a constant tension torque applied by the winding assembly (7) and contents of the blister pocket are available for dispensing via the flow channel (12). One the contents of the blister pockets are dispensed the outer cover (3) is rotated (closed) in the counter-clockwise direction. At this stage the second ridge (18) retracts the lever (2) back to initial state (FIG. 9C). Once the lever (2) reaches initial state, the second ridge (18) prevents further rotation of the outer cover (3) past the initial state of the lever (2). The lamination winds around the winding assembly (7). The wrapping wheel (11) rotates in the opposite direction of the blister cuvette (8) via the interim gear (10). The reel of blisters is separated from the blister advancing assembly via the inner housing assembly (4).

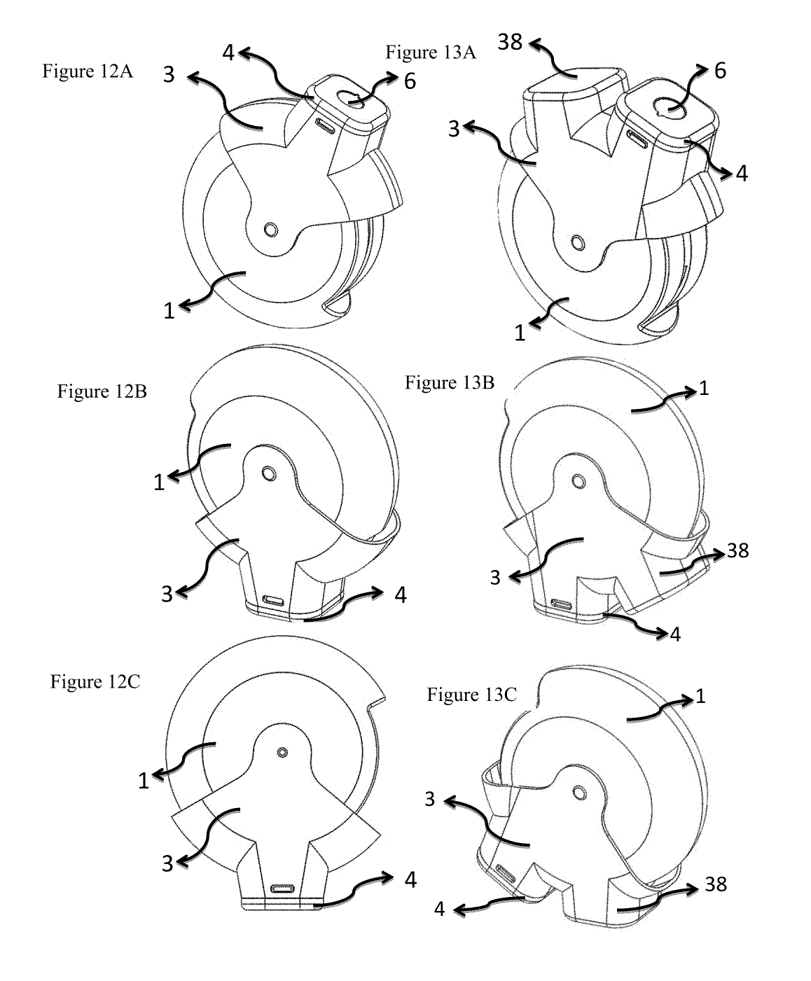

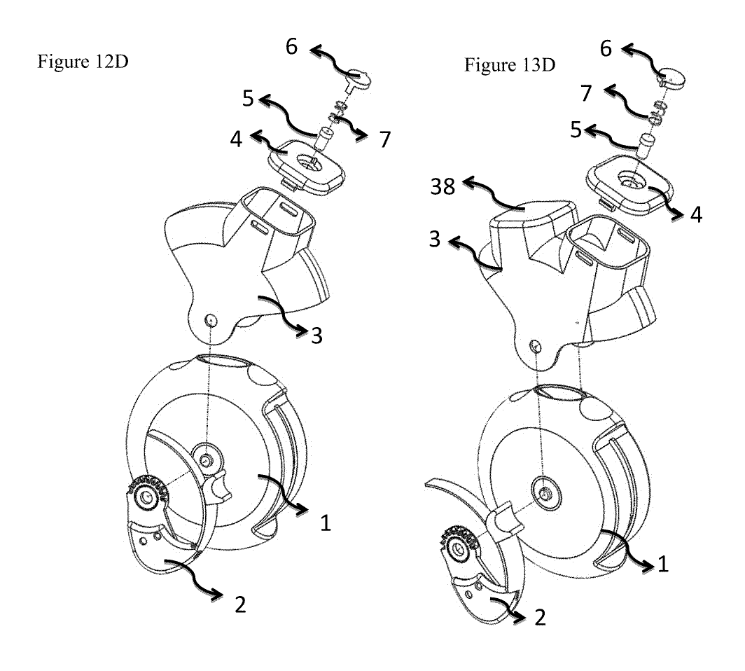

FIGS. 12A-12D and 13A-13D illustrate different views of the medicament dispenser according to another aspect of the disclosure. The medicament dispenser can include a diskette assembly (1) which houses a reel of blisters and the blister advancing mechanism. The blister advancing mechanism can be operated (or actuated) via the lever (2). The lever (2) can be operated (or actuated) via the outer cover (3).

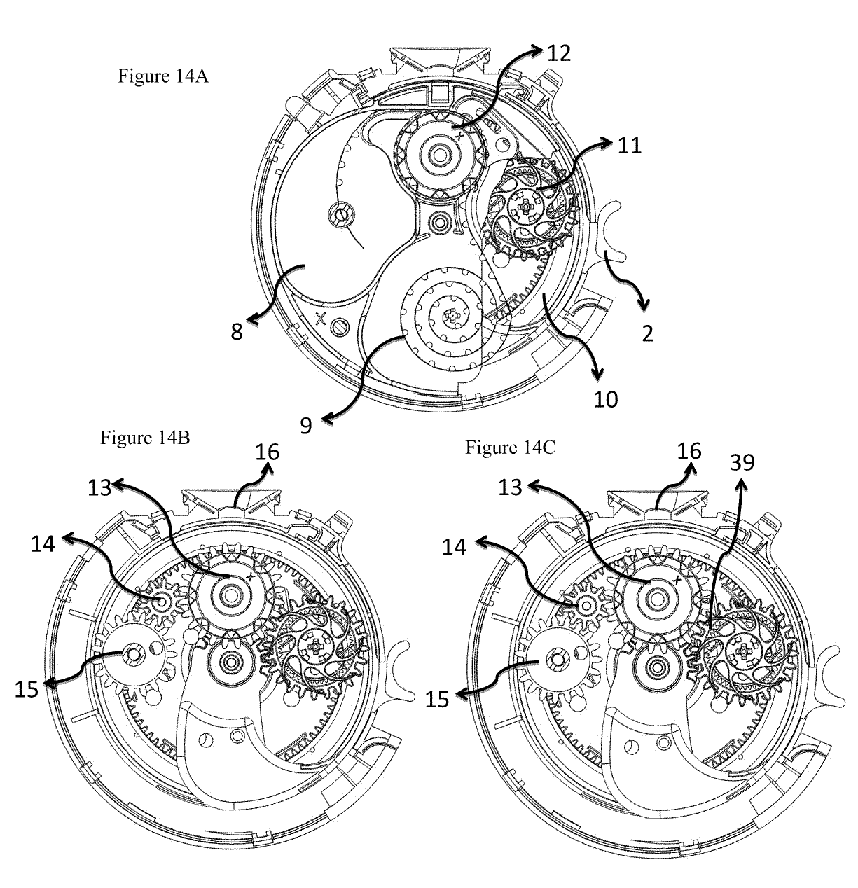

As shown in FIG. 14A, the diskette assembly (1) can house a reel of blisters (9) and the blister advancing mechanism. The reel of blisters (9) incorporates individual blister pockets comprising the powder, tablet, capsule, pellet or puck to be delivered by the medicament dispenser. The blister advancing mechanism can be operated (or actuated) via the lever (2). The lever (2) can be operated (or actuated) via the outer cover (3). The operation (or actuation) of lever can rotate the cuvette ratchet (13) by a discrete angular movement via the spur gears. The blister cuvette (12) can rotate synchronously and unidirectional with the cuvette ratchet (13), thereby advancing the blister pockets from the reel of blisters (9). Any movement by the lever (2) can lead to corresponding movement of the cuvette ratchet (13). The reel of blisters (9) that incorporates the blister pockets can be laminated at other end.

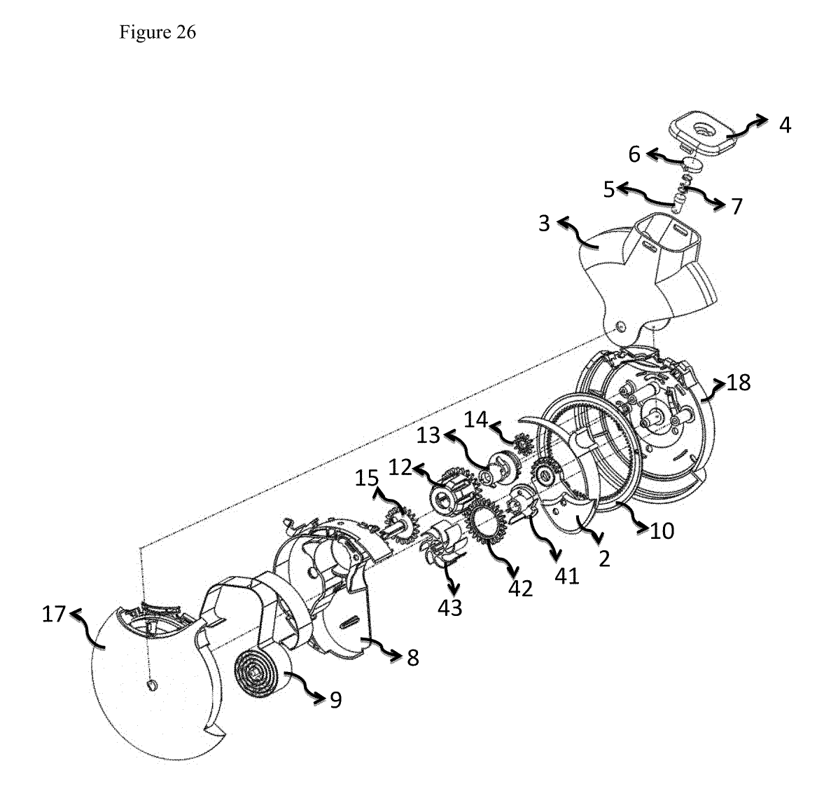

During operation of the medicament dispenser, the blister pockets align with the flow channel (16) of the diskette assembly. The lamination of the blister pockets can be peeled during each blister movement via a constant tension torque applied by the winding assembly (11). The winding assembly (11) can include the winder ratchet (41), winder gear (42) and the winder rotor (43) (see FIG. 26).

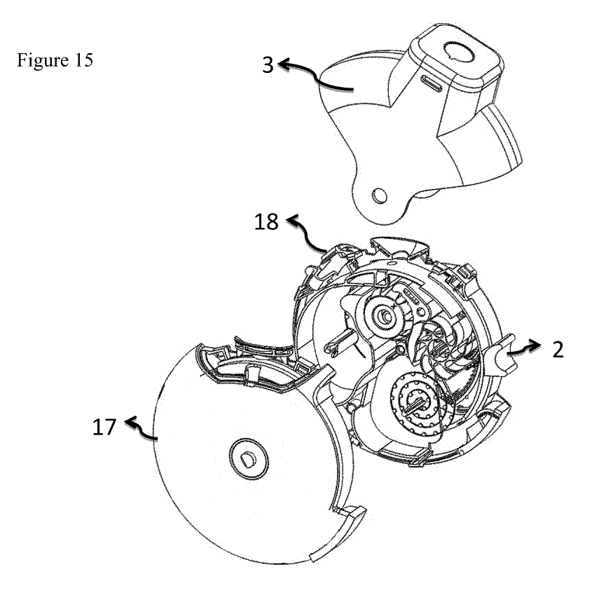

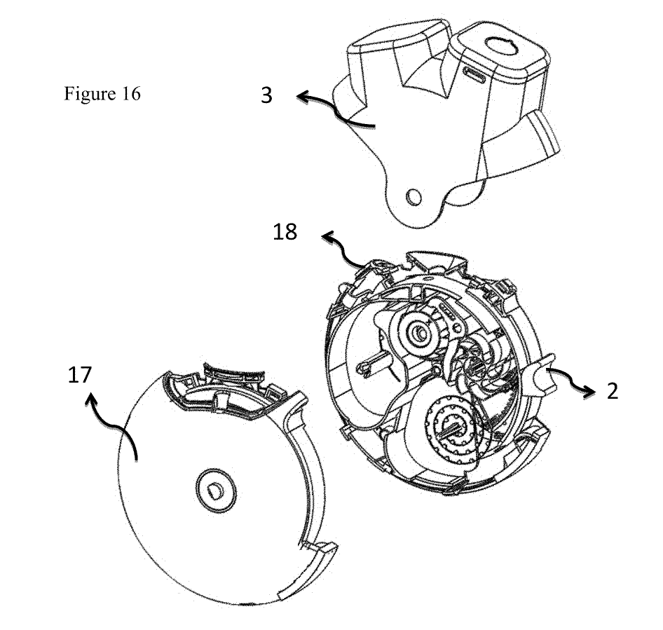

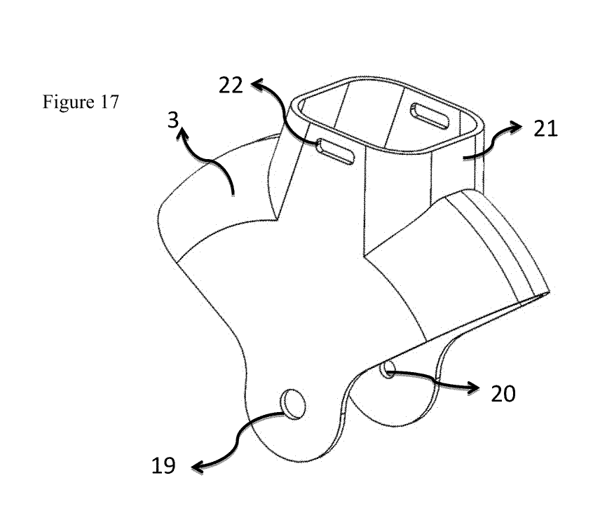

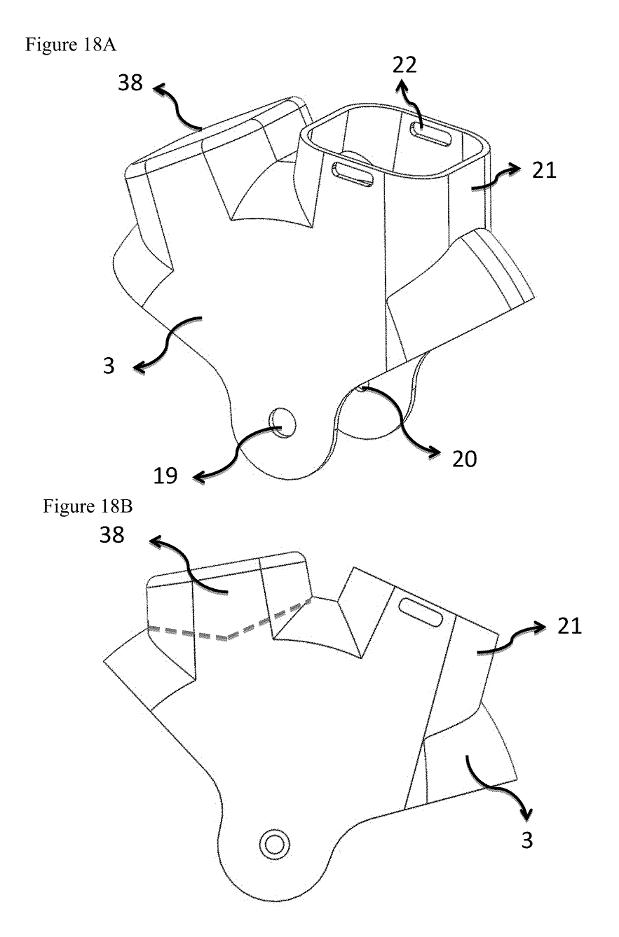

During operation of the medicament dispenser, the lamination winds around the winding assembly (11) (see FIG. 14A). By the same mechanism the blister pockets can be advanced via the wrapping wheel (15) (see FIGS. 14B and 14C). The wrapping wheel (15) can rotate in the opposite direction of the blister cuvette (12) via the interim gear (14). The blister reel can be separated from the blister advancing assembly via an inner housing assembly (8). The blister advancing mechanism can be protected by a top cover (17) (see FIGS. 15 and 16). The top cover (17) can lock with the base cover (18). The outer cover (3) pivots on the diskette assembly (1) which provides for the final assembly of the medicament dispenser.

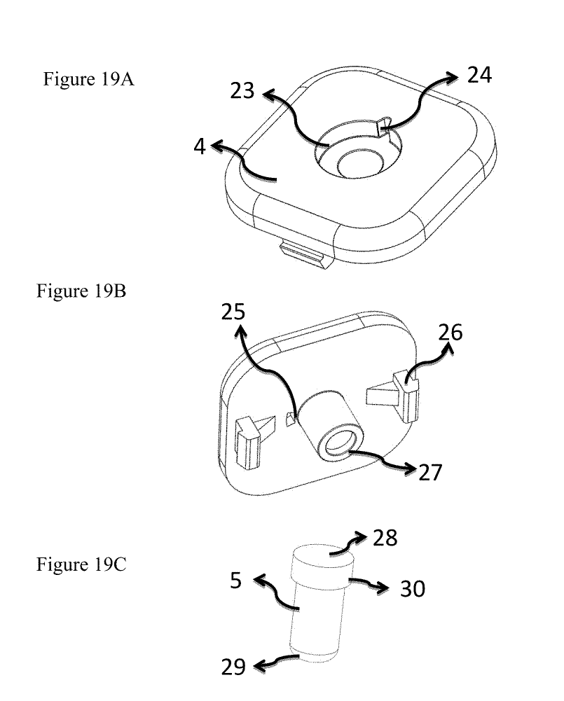

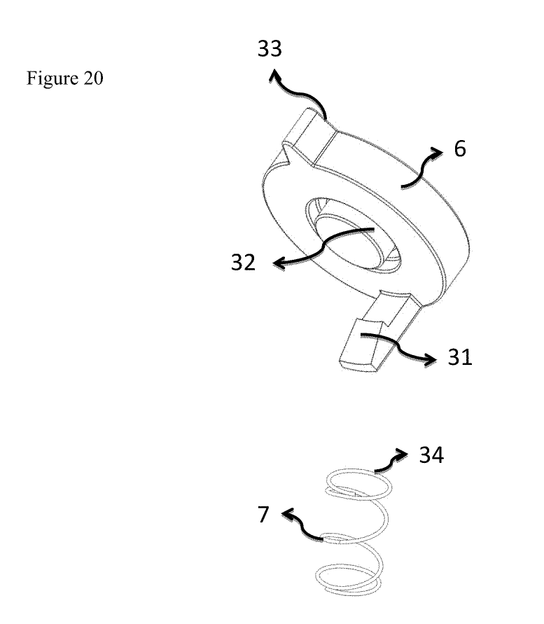

The outer cover (3) covers the flow channel and can operate (or actuate) the lever (2) via the plunger (5). The outer cover (3) can include a housing (21) (see FIGS. 17, 18A, and 18B) which incorporates the plunger (5), plunger cover (6) and the compression spring (7) (see FIGS. 19A-19C and 20). The elements within the housing (21) are protected by an outer cover end (4).

The outer cover end (4) protects the elements within the housing (21) of the outer cover (3). Notch (26) on the outer cover end (4) locks inside groove (22) and facilitates correct positioning of the outer cover end (4). The counter (23) can house the plunger cover (6) via a positioning groove (24). The notch (33) on the plunger cover (6) can engage the positioning groove (24) and lock the plunger cover (6) in place by locking notch (31) on plunger cover (6) with groove 25. The bearing (27) provides a bearing for the plunger seat (30). The plunger (5) can be spring loaded via a compression spring (7). The compression spring (7) can rest on surface (28) of the plunger (5).

Plunger cover (6) can protect the plunger (5) and provide a guide to the compression spring (7) (see FIG. 20). The guiding groove (32) can act as a guiding groove for the compression spring (7) and facilitates preloading of the compression spring. Notch (31) can lock with the outer cover end (4) via groove (25) on outer cover end (4). Notch (33) can be used to guide the plunger cover (6) during assembly. The flat end (34) of the compression spring can be positioned within the guiding groove (32) of the plunger cover (6).

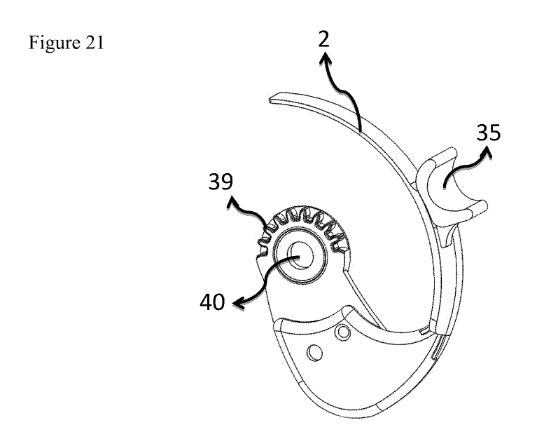

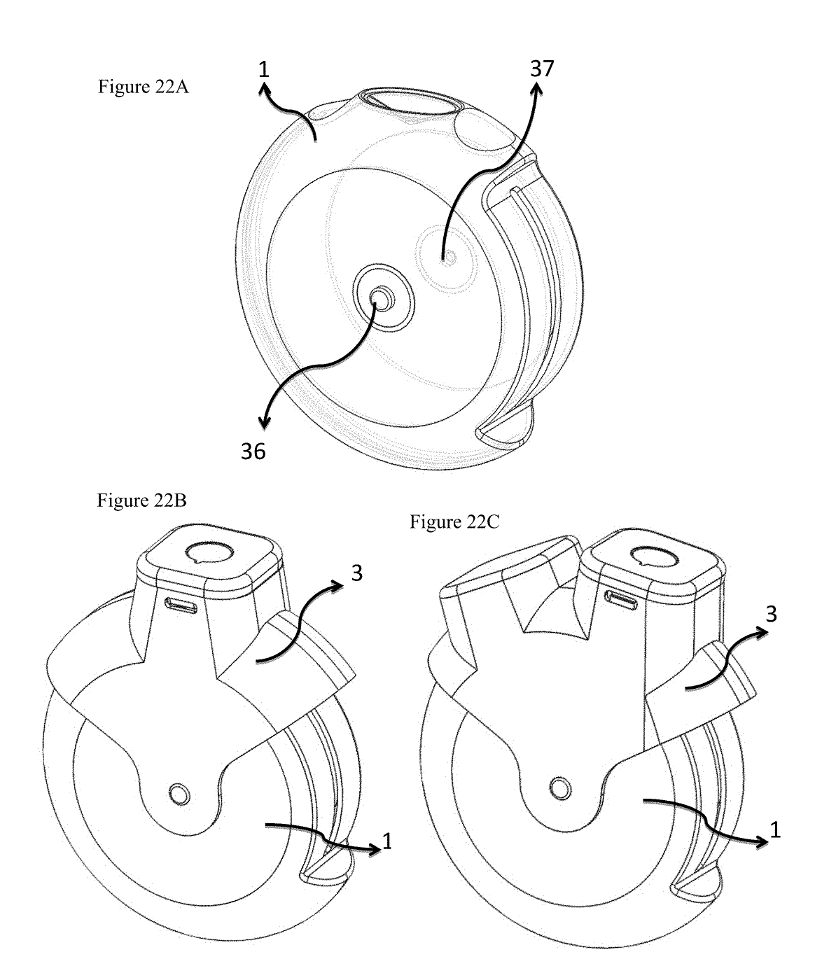

When the outer cover (3) is in the initial (rest) position, the plunger nose (29) pushes on the diskette assembly. When the outer cover (3) is rotated during operation (actuation), the plunger nose (29) pushes on the driver (35) of the lever (2) (FIG. 21). The compression spring (7) provides the necessary pre load on the driver (35) of the lever (2) via the plunger (5) and plunger nose (29). Compression Spring (7) pushes the plunger (5) to engage plunger nose (29) with the driver (35) on the lever (2).

The outer cover (3) can include mounting holes (19) and (20) that lock with respective studs 36 and 37 on the diskette assembly (1) (FIG. 17). In some embodiments, the outer cover (3) (FIGS. 18A and 18B) can include compartment (38) that is a dedicated space to accommodate for a power source and electronic instruments. The studs 36 and 37 on the diskette assembly (1) provide a hinge about which the outer cover (3) can rotate and engage the lever (2). Once the plunger nose (29) engages with the lever (2), the outer cover (3) can move synchronously with the lever (2). Any movement of the outer cover (3) can lead to corresponding movement of the lever (2). The lever teeth (39) mate with the blister advancing mechanism within the diskette assembly (1) to advance the reel of blisters (9) and the dose ring (10).

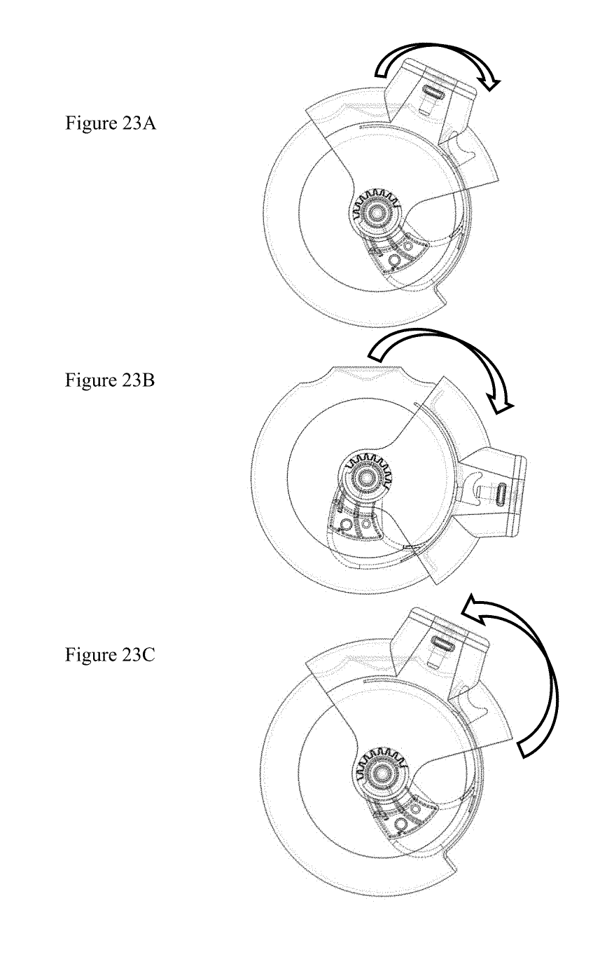

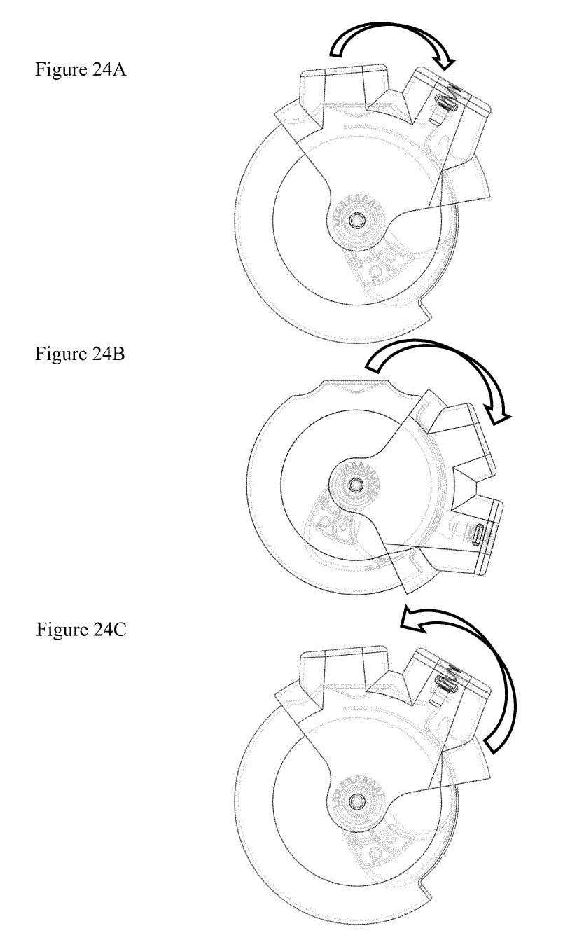

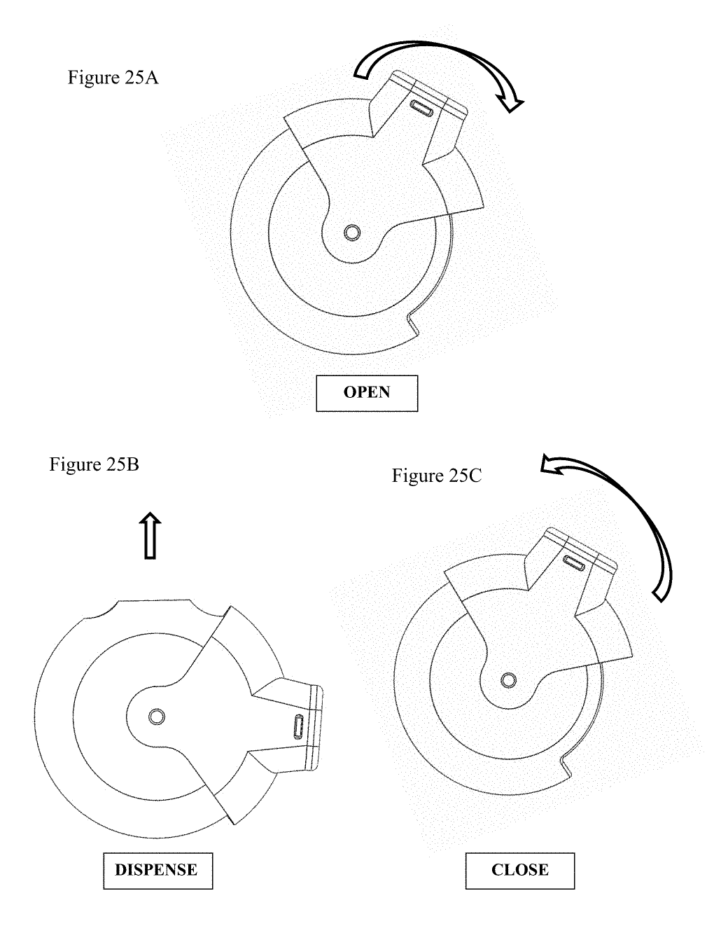

The mechanism of operation of the medicament dispenser is presented in FIGS. 23A-23C, 24A-24C, and 25A-25C. The inhaler assembly can include a diskette assembly, lever and an outer cover. The blister advancing mechanism is located within the diskette assembly. In the initial (rest) state the outer cover (3) covers the flow channel (16) of the diskette assembly (1). In this position the plunger nose (29) can push on the diskette assembly (1). The lever (2) is in a rest state before outer cover is operated. When the outer cover (3) of the medicament inhaler is rotated clockwise (opened) the plunger nose (29) pushes on the driver (35) of the lever (2). As the outer cover (3) is rotated, the lever (2) can rotate along with the plunger nose (29). The actuation of lever can rotate the cuvette ratchet (13) by a discrete angular movement via the spur gears. The blister cuvette (12) can rotate synchronously with the cuvette ratchet (13) uni-directionally, thereby advancing the blister pockets from the reel of blisters (9). Any movement of the outer cover (3) and/or lever (2) can lead to corresponding movement of the blister cuvette (12) and/or cuvette ratchet (13). At this stage the blister pockets align with the flow channel (16) of the diskette assembly. The lamination of the blister pockets can be peeled via a constant tension torque applied by the winding assembly (11) and contents of the blister pocket are available for dispensing via the flow channel (16). One the contents of the blister pockets are dispensed, the outer cover (3) can be rotated (closed) in the counter-clockwise direction. At this stage the plunger nose (29) can retract the lever (2) back to initial state and comes in contact with the diskette assembly (1).

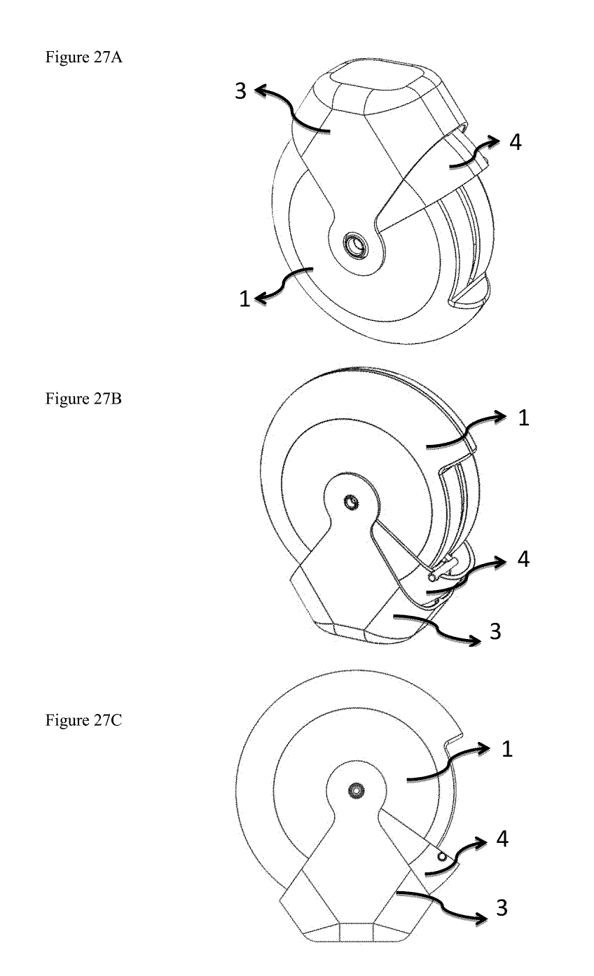

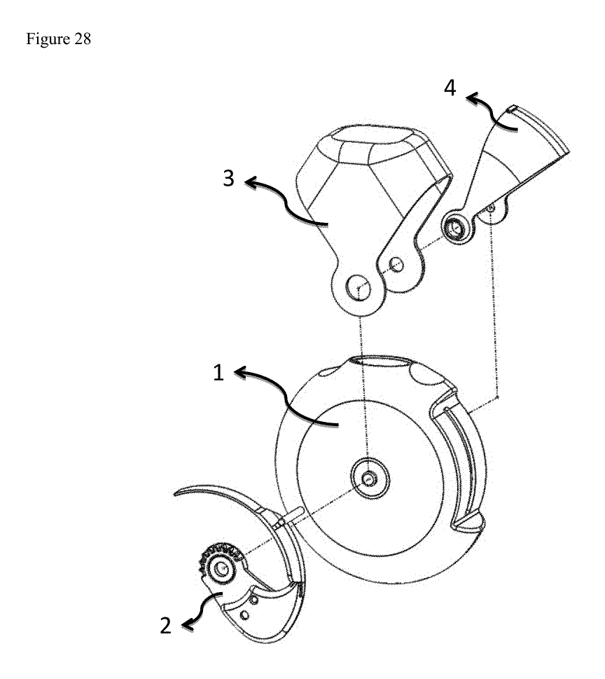

FIGS. 27A-27C illustrate different views of the medicament dispenser according to another aspect of the disclosure. The medicament dispenser can include a diskette assembly (1) which houses a reel of blisters (not shown) and the blister advancing mechanism. The blister advancing mechanism is operated (or actuated) via the lever (2) (see FIG. 28). The lever (2) can be operated (or actuated) via the outer cover (3) and the inner cover (4).

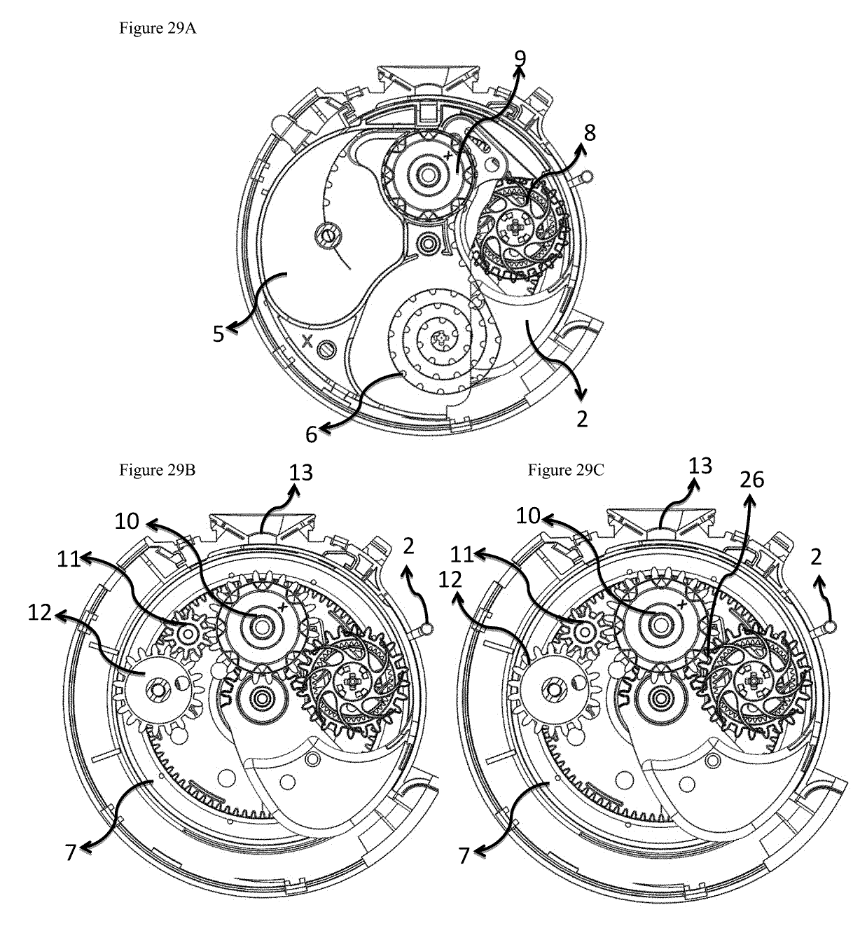

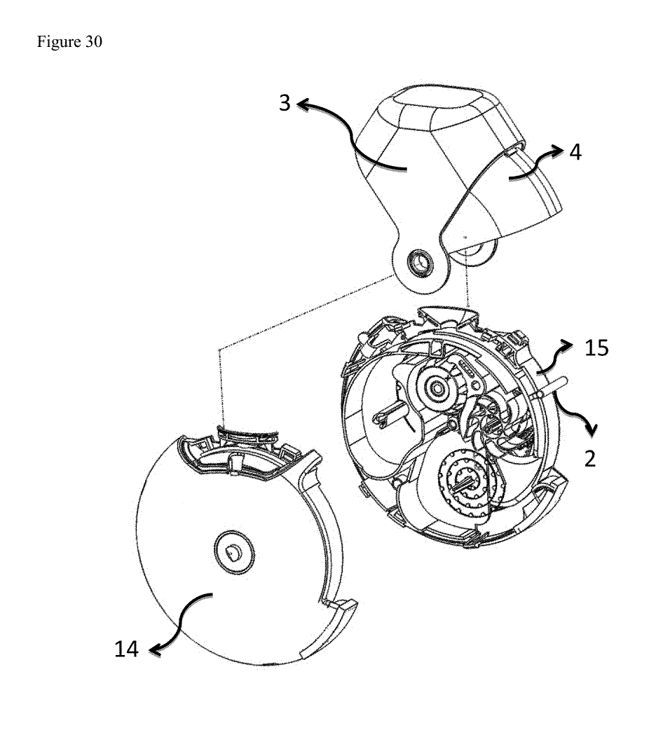

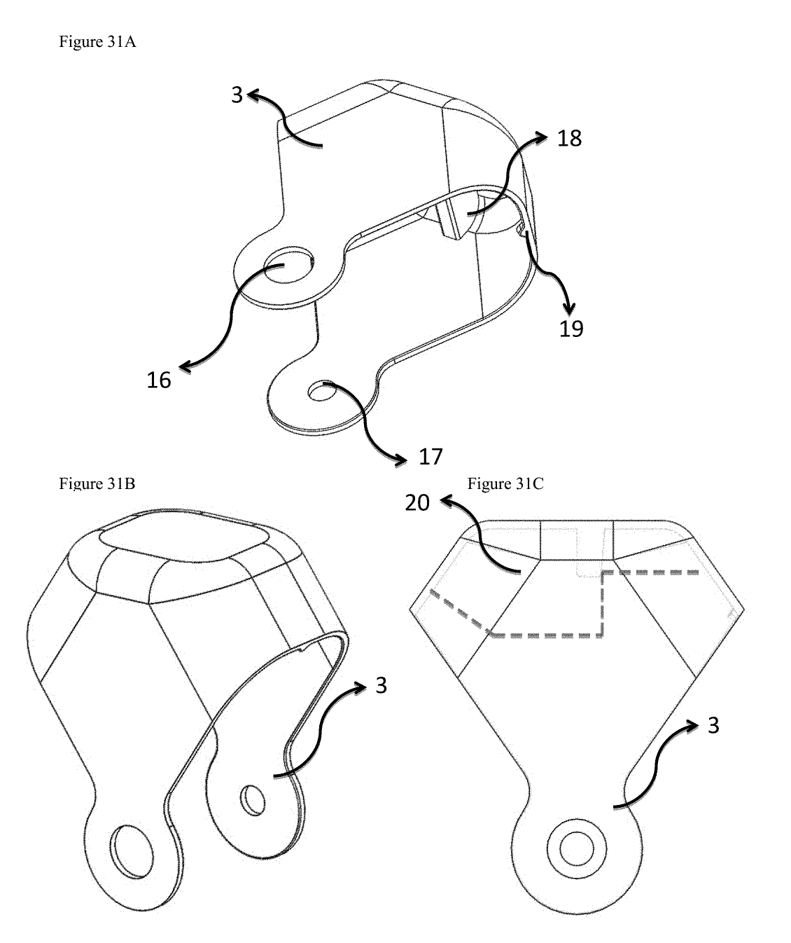

As shown in FIGS. 29A-29C, the diskette assembly (1) can house a reel of blisters (6) and the blister advancing mechanism. The reel of blisters (6) incorporates individual blister pockets having a powder, tablet, capsule, pellet or puck to be delivered by the medicament dispenser. The blister advancing mechanism is operated (or actuated) via the lever (2). The lever (2) is locked with the inner cover (4) and rotates along with it. The inner cover (4) can be operated (or actuated) by the outer cover (3). The operation (or actuation) of lever (2) can rotate the cuvette ratchet (10) by a discrete angular movement via the spur gears. The blister cuvette (9) can rotate synchronously and unidirectional with the cuvette ratchet (10), thereby advancing the blister pockets from the reel of blisters (6). Any movement by the lever (2) can lead to corresponding movement of the cuvette ratchet (10). The reel of blisters (6) that incorporates the blister pockets is laminated at other end.

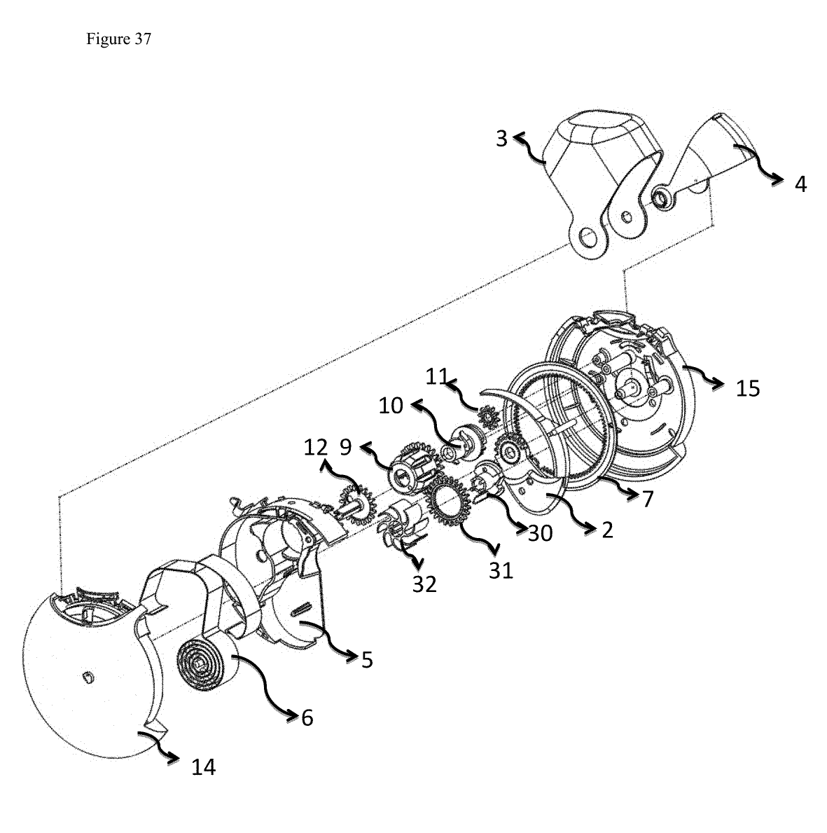

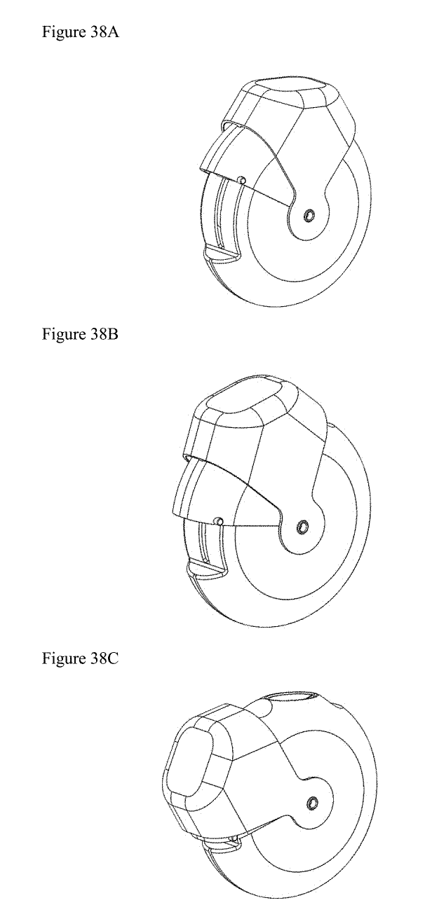

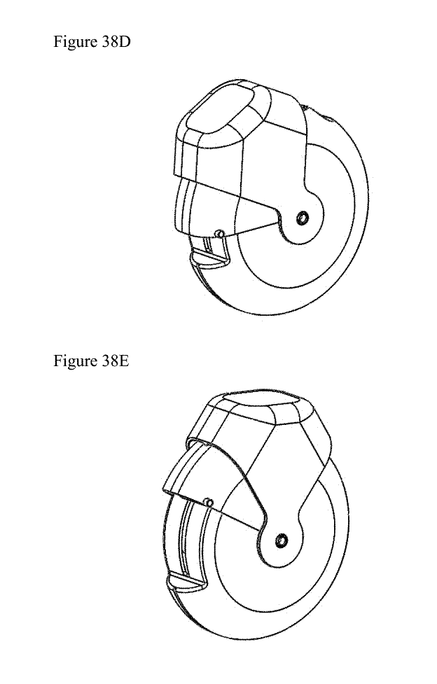

During operation of the medicament dispenser the blister pockets align with the flow channel (opening) (13) of the diskette assembly. The lamination of the blister pockets can be peeled during each blister movement via a constant tension torque applied by the winding assembly (8). The winding assembly (8) can include the winder ratchet (30), winder gear (31) and the winder rotor (32) (see FIG. 37).

During operation of the medicament dispenser the lamination winds around the winding assembly (8) (see FIG. 29A). By the same mechanism the blister pockets are advanced via the wrapping wheel (12) (see FIGS. 29B and 29C). The wrapping wheel (12) rotates in the opposite direction of the blister cuvette (9) via the interim gear (11). The blister reel can be separated from the blister advancing assembly via an inner housing assembly (5).

After the operation (or actuation) is completed, the outer cover (3) can be rotated to cover the flow channel (opening) (13). The motion of the outer cover (3) can rotate inner cover (4) and hence the lever (2) is locked with the inner cover (4) to home position without advancing the blister. During the operation (or actuation) of the lever (2), a gear attached to the wrapping wheel (12) can rotate the mechanical dose ring (7) to display the number of doses consumed by the user.

The blister advancing mechanism is protected by a top cover (14) (FIG. 30). The top cover (14) locks with the base cover (15). The outer cover (3) and inner cover (4) pivot on the diskette assembly (1) which provides for the final assembly of the medicament dispenser.

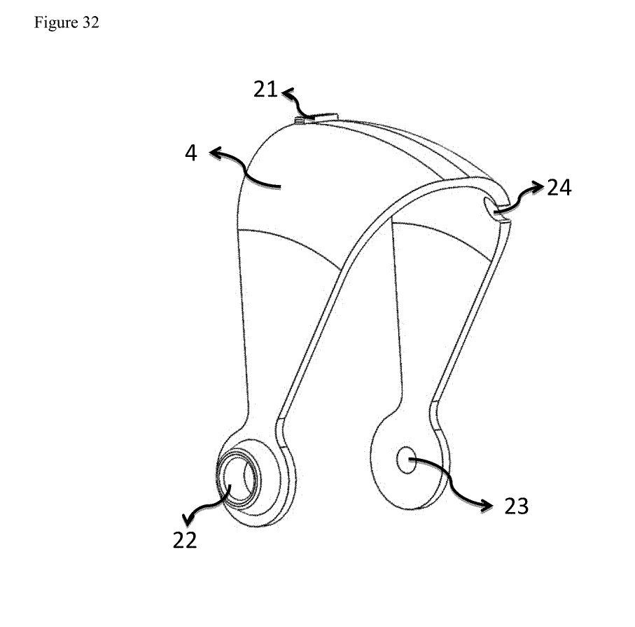

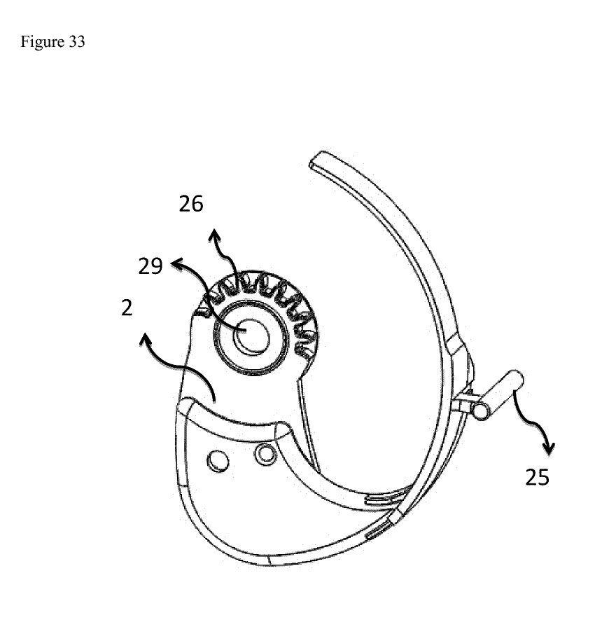

The outer cover (3) (FIGS. 31A-31C) covers the flow channel and operates (or actuates) the lever (2) via the inner cover (4). The first ridge (18) on the outer cover (3) can push on the inner cover (4) (FIG. 32) during operation (or actuation) thereby actuating the lever (2). The second ridge (19) on the outer cover (3) can pull on the protrusion (21) on the inner cover (4) during travel back to home (rest) position. Features 16 and 17 on the outer cover (3), pivot around the hinges 22 and 23 on the inner cover (4). In some embodiments of the outer cover (3) (FIG. 31C) includes a compartment (20), which is a dedicated space to accommodate for a power source and electronic instruments.

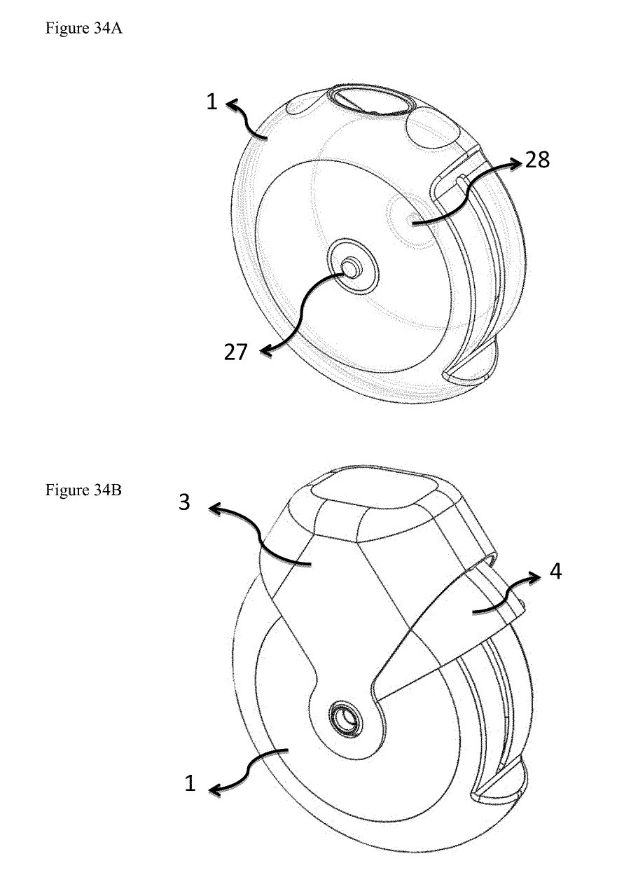

The inner cover (4) can include of a protrusion (21) that engages with the second ridge (19) on the outer cover (3) during retraction to the home (rest) position. The inner cover (4) can include a groove (24) that locks the lever bar (25) of the lever (2). This allows the inner cover (4) and the lever (2) to rotate in phase. Features 22 and 23 on the inner cover (4) can act as hinges to the features 16 and 17 on the outer cover (3). Features 22 and 23 on the inner cover (4) can also pivot about studs 27 and 28 on the diskette assembly (1) (FIGS. 34A and 34B).

The lever (2) (FIG. 33) can include lever bar (25) which locks with the groove (24) on the inner cover (4). During operation (or actuation), the outer cover (3) can contact the inner cover (4) to rotate both the inner cover (4) and the lever (2). Similarly, during retraction, the outer cover (3) can contact the inner cover (4) via protrusion 21 on the inner cover (4) and the second ridge (19) on the outer cover (3), while rotating the lever (2) about the center on each occasion. The lever teeth (26) on the lever can mate with the blister advancing mechanism inside the diskette assembly (1) to advance the reel of blisters (6) and the mechanical indicator dose ring (7).

The diskette assembly (1) (FIGS. 34A and 34B) can house the reel of blisters (6) and the blister advancing mechanism. The blister advancing mechanism is actuated via the lever (2). The studs 27 and 28 can provide a hinge for the inner cover (4) which locks with the lever (2) to actuate the blister advancing mechanism inside the diskette assembly (1).

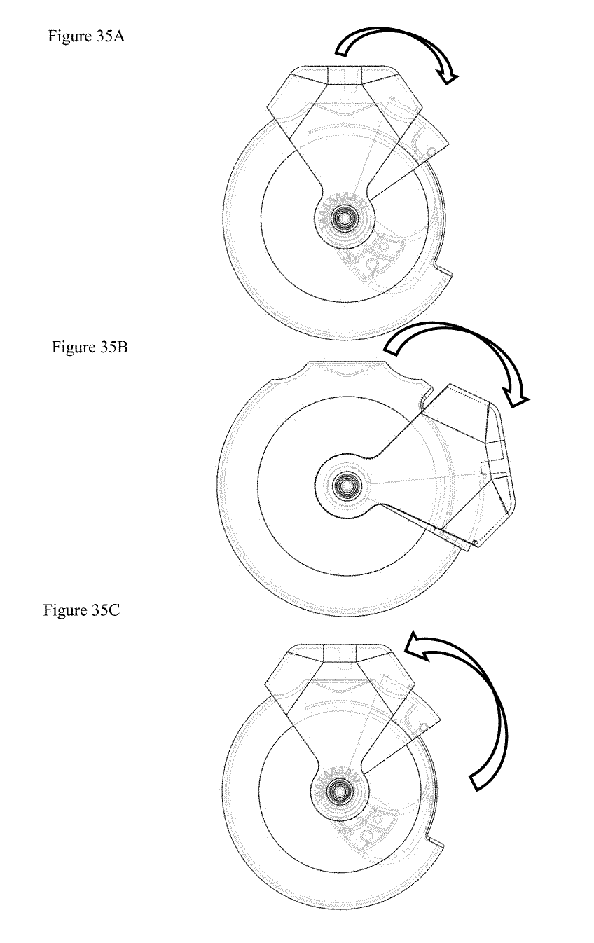

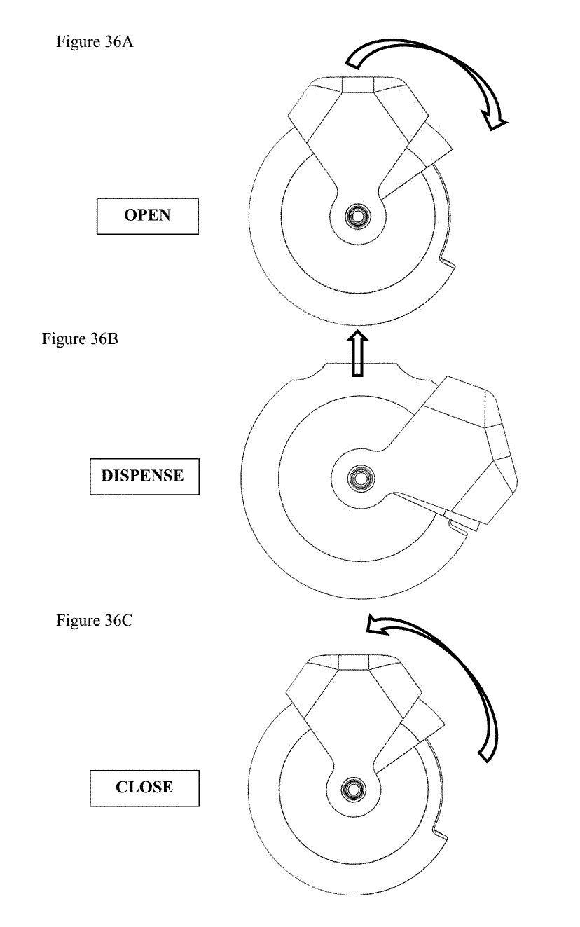

The mechanism of operation of the medicament dispenser is presented in FIGS. 35A-35C and 36A-36C. The inhaler assembly can include of a diskette assembly, lever and an outer cover. The blister advancing mechanism is located within the diskette assembly. In the initial (rest) state the outer cover (3) covers the flow channel (13) of the diskette assembly (1). In this position the lever (2) is in a rest state before outer cover is operated. When the outer cover (3) of the medicament inhaler is rotated clockwise (opened), it engages with the inner cover (4). During this operation the groove (24) on the inner cover (4) locks with the lever bar (25) of the lever (2). As the outer cover (3) and inner cover (4) are rotated, the lever (2) rotates actuating the blister advancing mechanism inside the diskette assembly (1). The actuation of lever rotates the cuvette ratchet (10) by a discrete angular movement via the spur gears. The blister cuvette (9) rotates synchronously with the cuvette ratchet (10) uni-directionally, thereby advancing the blister pockets from the reel of blisters (6). At this stage the blister pockets align with the flow channel (13) of the diskette assembly. The lamination of the blister pockets is peeled via a constant tension torque applied by the winding assembly (8) and contents of the blister pocket are available for dispensing via the flow channel (13). One the contents of the blister pockets are dispensed the outer cover (3) is rotated (closed or retracted) in the counter-clockwise direction. At this stage the second ridge (19) on the outer cover (3) pulls on the protrusion (21) on the inner cover (4) during travel back to home (rest) position. This action retracts the lever (2) back to initial state (rest state).

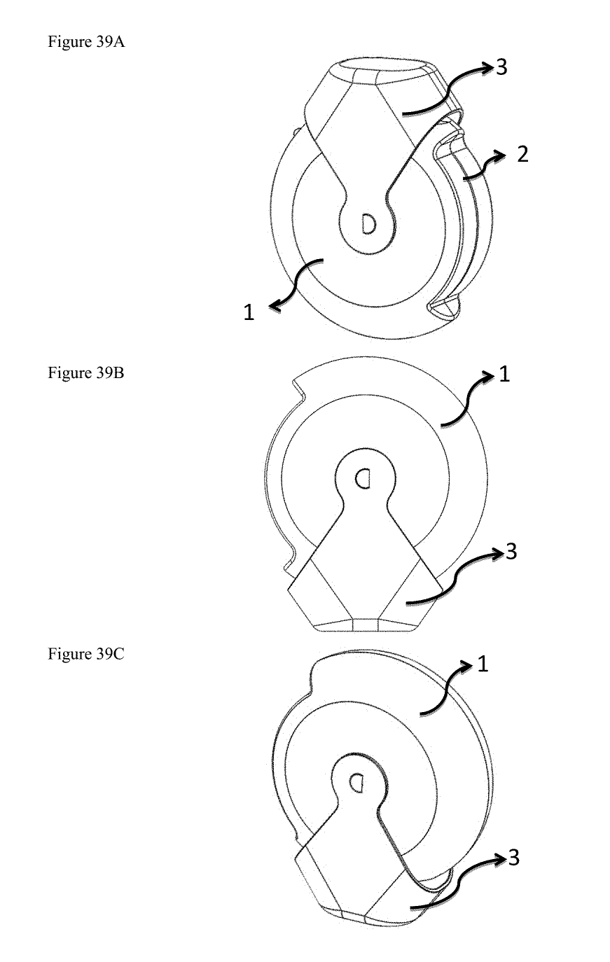

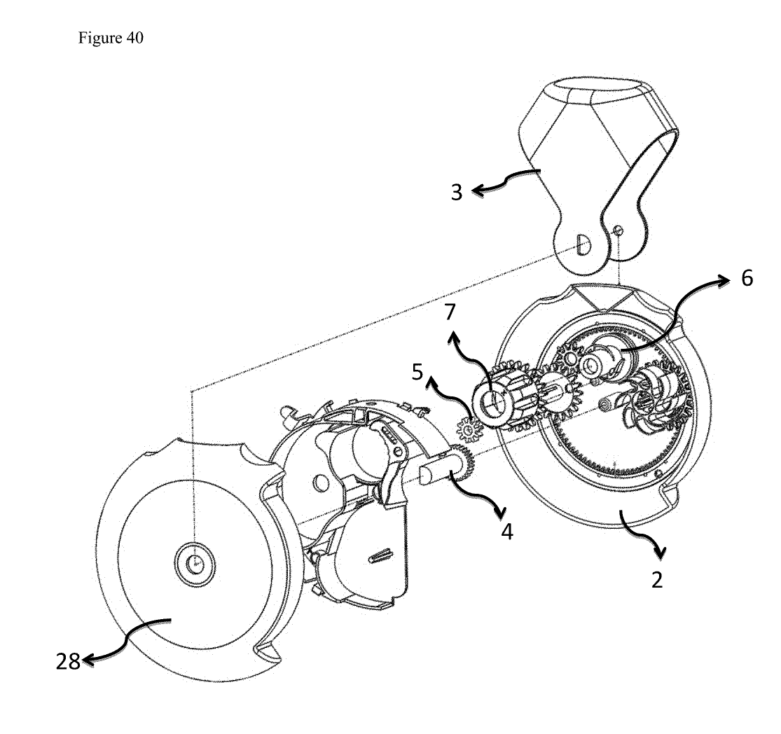

FIGS. 39A-39C illustrate different views of the medicament dispenser according to another aspect of the disclosure. The medicament dispenser can include a diskette assembly (1) which houses a reel of blisters and the blister advancing mechanism. The blister advancing mechanism is operated (or actuated) via the outer cover (3).

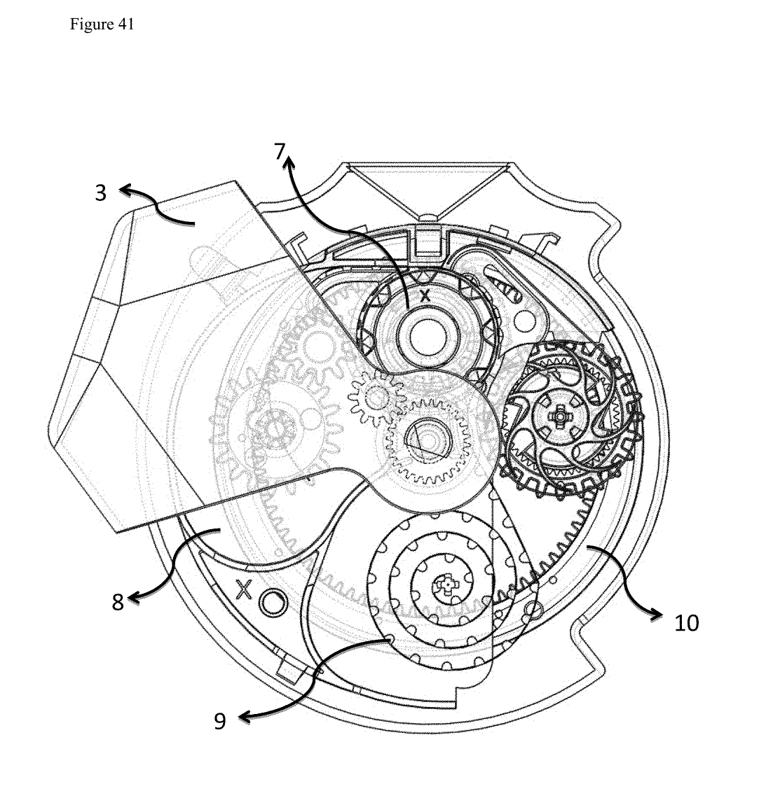

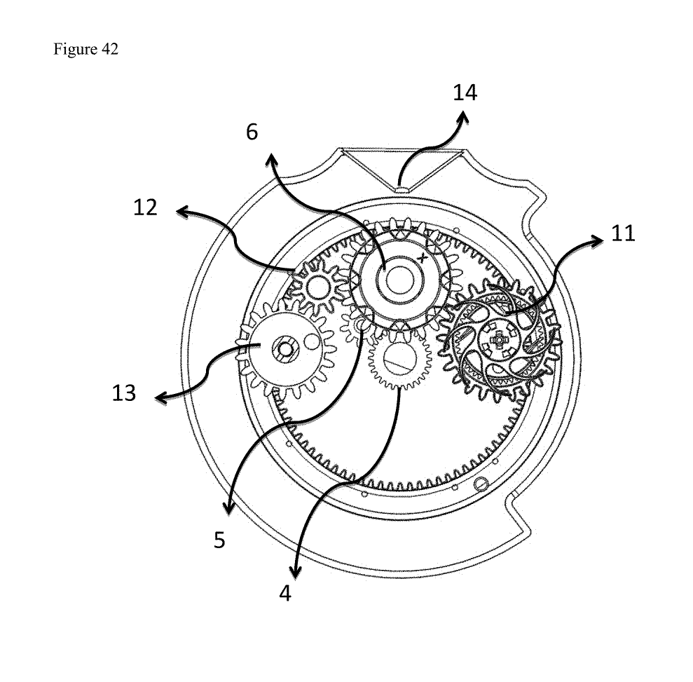

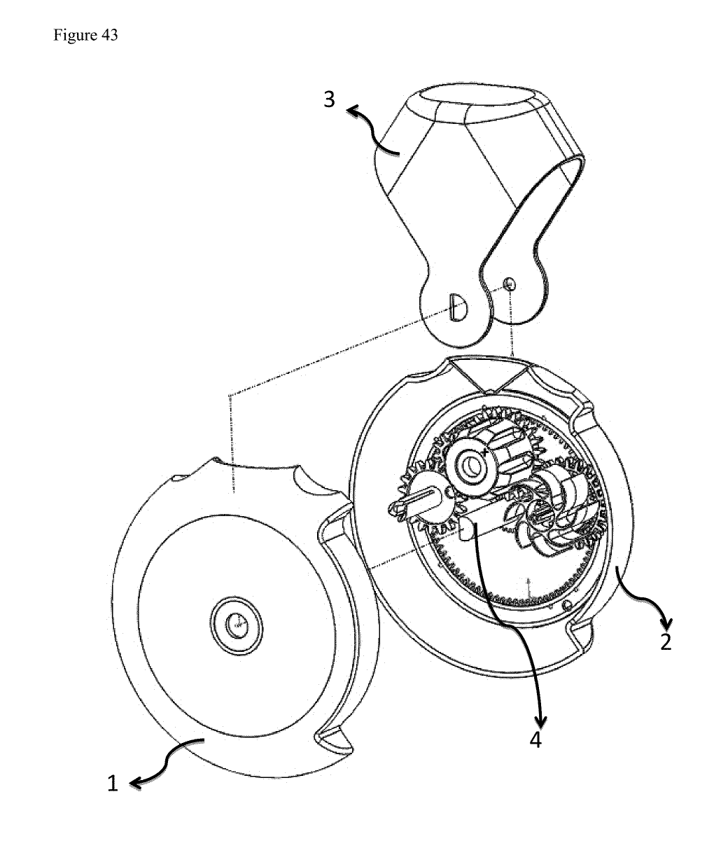

As shown in FIG. 41, the diskette assembly (1) can house a reel of blisters (9) and the blister advancing mechanism. The reel of blisters (9) incorporates individual blister pockets having the powder, tablet, capsule, pellet or puck to be delivered by the medicament dispenser. The blister advancing mechanism is operated (or actuated) via the outer cover (3). The outer cover (3) is keyed with the boss on the central gear (4) and can rotate along with it. The central gear (4) can mate with idle gear (5) which is used to change to direction of rotation of the cuvette ratchet (6). The actuation of outer cover (3) can rotate the cuvette ratchet (6) via the idle gear (5) and central gear (4). The blister cuvette (7) can rotate synchronously and unidirectional with the cuvette ratchet (6), thereby advancing the blister pockets from the reel of blisters (9). Any movement of the outer cover (3) can lead to corresponding movement of the cuvette ratchet (6). The reel of blisters (9) that incorporates the blister pockets is laminated at other end.

During operation of the medicament dispenser the blister pockets align with the flow channel (opening) (14) of the diskette assembly. The lamination of the blister pockets can be peeled during each blister movement via a constant tension torque applied by the winding assembly (11).

During operation of the medicament dispenser the lamination winds around the winding assembly (11) (see FIG. 42). By the same mechanism the blister pockets are advanced via the wrapping wheel (13). The wrapping wheel (13) rotates in the opposite direction of the blister cuvette (7) via the interim gear (12). The blister reel is separated from the blister advancing assembly via an inner housing assembly (8).

After the operation (or actuation) is completed, the outer cover (3) can be rotated to cover the flow channel (opening) (14). The motion of the outer cover (3) can rotate the cuvette ratchet (6) via the idle gear (5) and the central gear (4) without advancing the blister. During the operation (or actuation) of the outer cover (3), a gear attached to the wrapping wheel (13) rotates the mechanical dose ring (10) to display the number of doses consumed by the user (see FIG. 41).

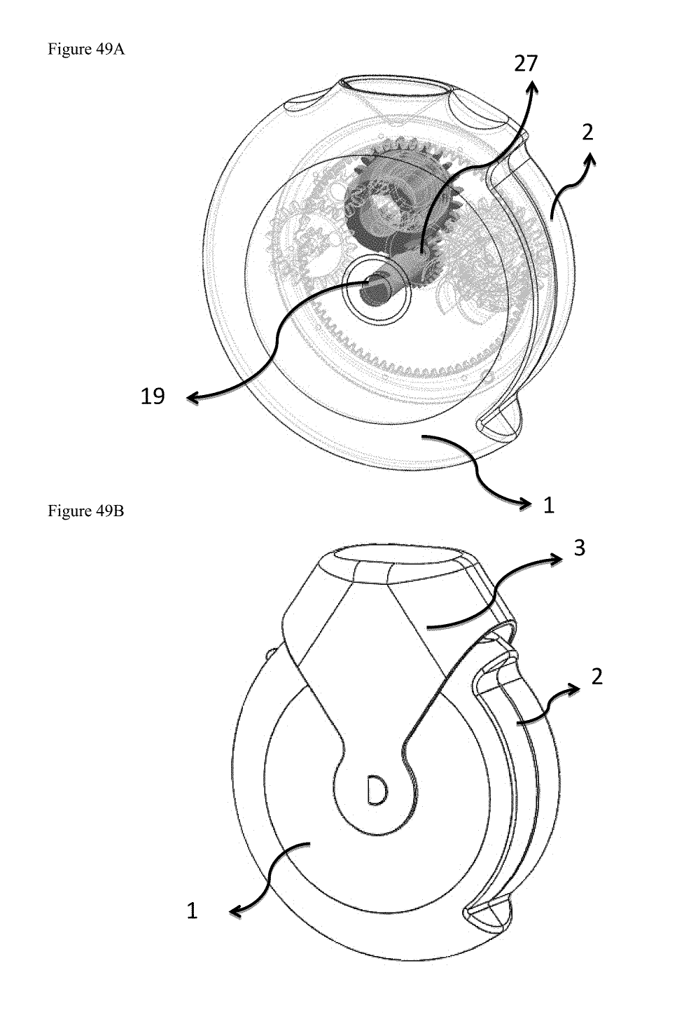

The blister advancing mechanism can be protected by a top cover (28) (see FIG. 43). The top cover (28) locks with the base cover (2) (FIGS. 49A and 49B). The outer cover (3) keys on the boss of central gear (4) located on the boss at the center of the base cover (2). The blister advancing mechanism is located within the top cover (28) and the base cover (2) forming the diskette assembly. The outer cover (3) pivots on the diskette assembly (1) which provides for the final assembly of the medicament dispenser.

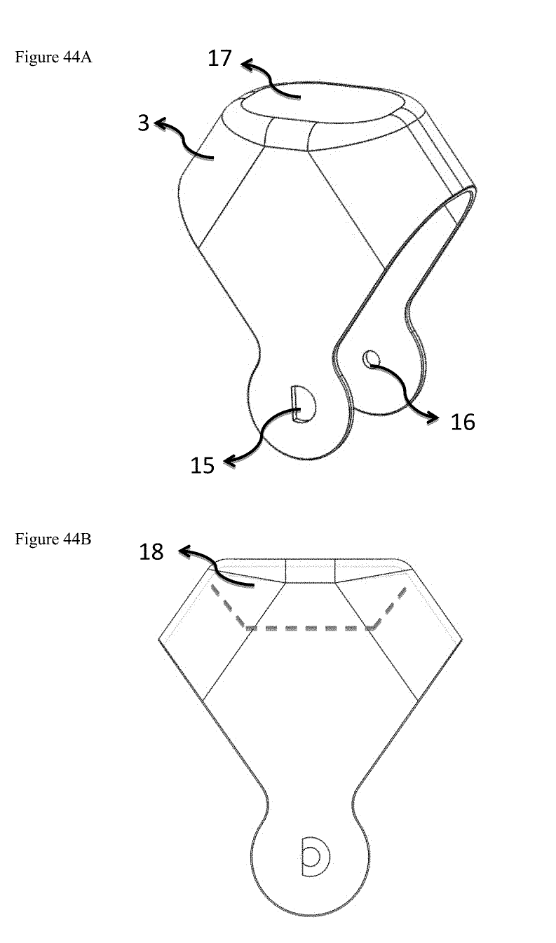

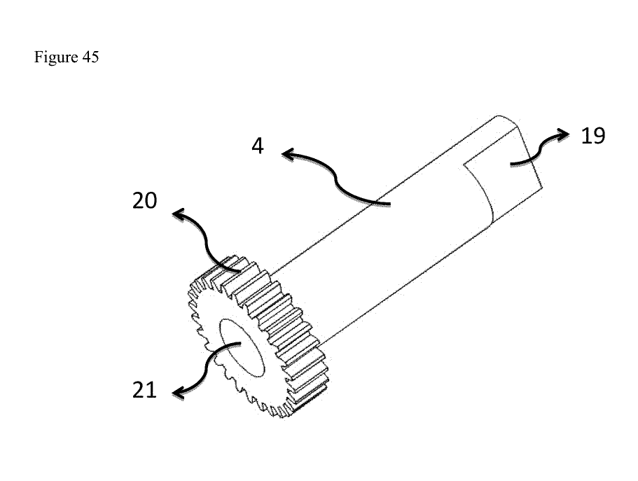

The outer cover (3) (FIGS. 44A and 44B) covers the flow channel. Feature 16 on the outer cover (3) locks with the stud (27) on the base cover (2). Feature 15 on the outer cover (3) provides a key way for the key on the boss (19) of the central gear (4) (see FIGS. 44A, 44B, and 45). The torque from the outer cover (3) can be transmitted to the central gear (4) via the key way (15). The key way (15) allows a synchronous motion of the outer cover (3) with the central gear (4). Any movement by the outer cover (3) can lead to corresponding movement of the central gear (4). The flat surface (17) on the outer cover (3) can be used to place the outer cover in a vertical position. In some embodiments of the outer cover (3) (FIGS. 44A and 44B) the compartment (18) can include a dedicated space to accommodate for a power source and electronic instruments.

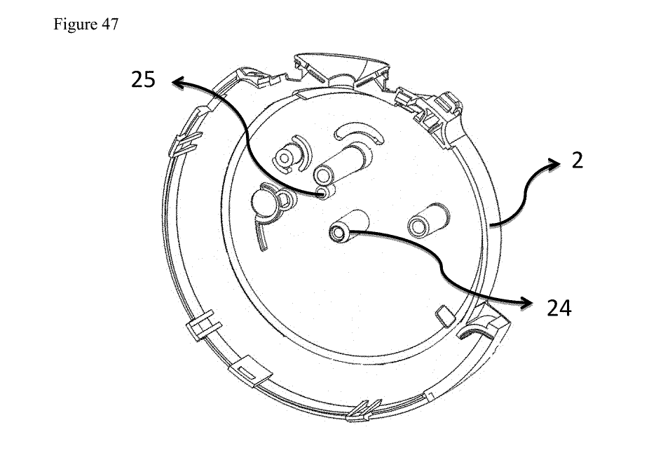

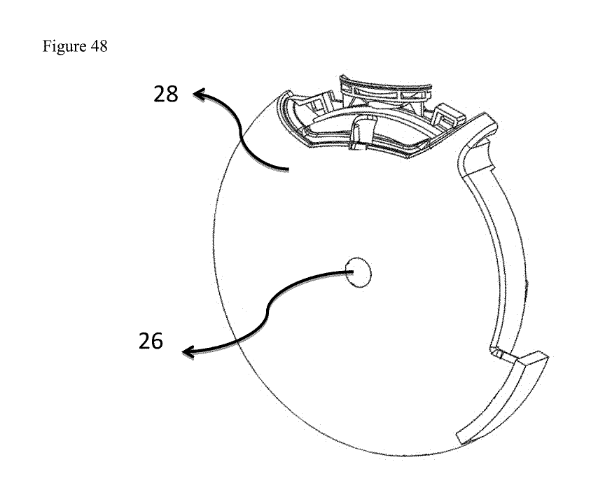

The central gear (4) (FIG. 45) includes a boss (19) that can pass through an opening (26) of the top cover (28) (FIG. 48) and can mate with the key way (15) of the outer cover (3). The central gear can ride over the boss (24) (FIG. 47) on the base cover (2) via bearing (21). The teeth (20) on the central gear (4) can mate with the idle gear (5) to drive the blister advancing mechanism.

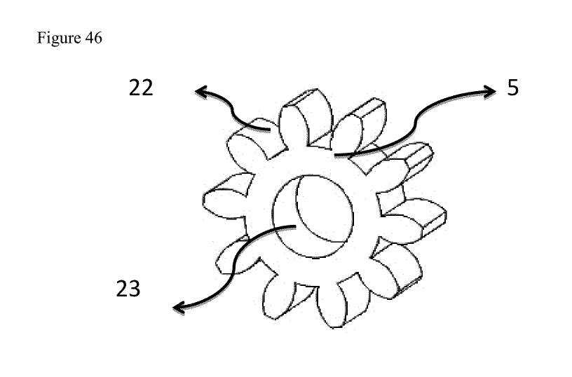

The idle gear (5) (FIG. 46) can ride over a stud (25) on the base cover (2). Feature 23 provides a bearing for the stud (25). The gear teeth (22) on the idle gear (5) mate with teeth (20) on the central gear (4) and teeth on the cuvette ratchet (6). The idle gear (5) enables the cuvette ratchet (6) to rotate in direction opposite of the central gear (4).

The base cover (2) (FIG. 47) can include stud (24) and stud (25) that enable the idle gear (5) and the central gear (4) to ride on studs 24 and 25, respectively. The cuvette ratchet (6), winding assembly (11), interim gear (12), wrapping wheel (13) ride on the other studs in the assembly. These components together can form the blister advancing mechanism.

The top cover (28) (FIG. 48) can lock with the separate locking features on the base cover (2) and protects the blister advancing mechanism. The key way (19) on the boss of the central gear (4) can pass through the opening (26) on the top cover (28) to mate with key (15) on the outer cover (3).

The diskette assembly (FIGS. 49A and 49B) can include the top cover (28) and the base cover (2) and houses the reel of blisters (6) and the blister advancing mechanism within the top cover (28) and base cover (2). Stud 27 can provide a hinge for the outer cover (3). The boss (19) on the central gear (4) can lock with the key way (15) of the outer cover (3) and can rotate along with the outer cover (3). Rotation of the outer cover (3) can actuation the blister advancing mechanism.

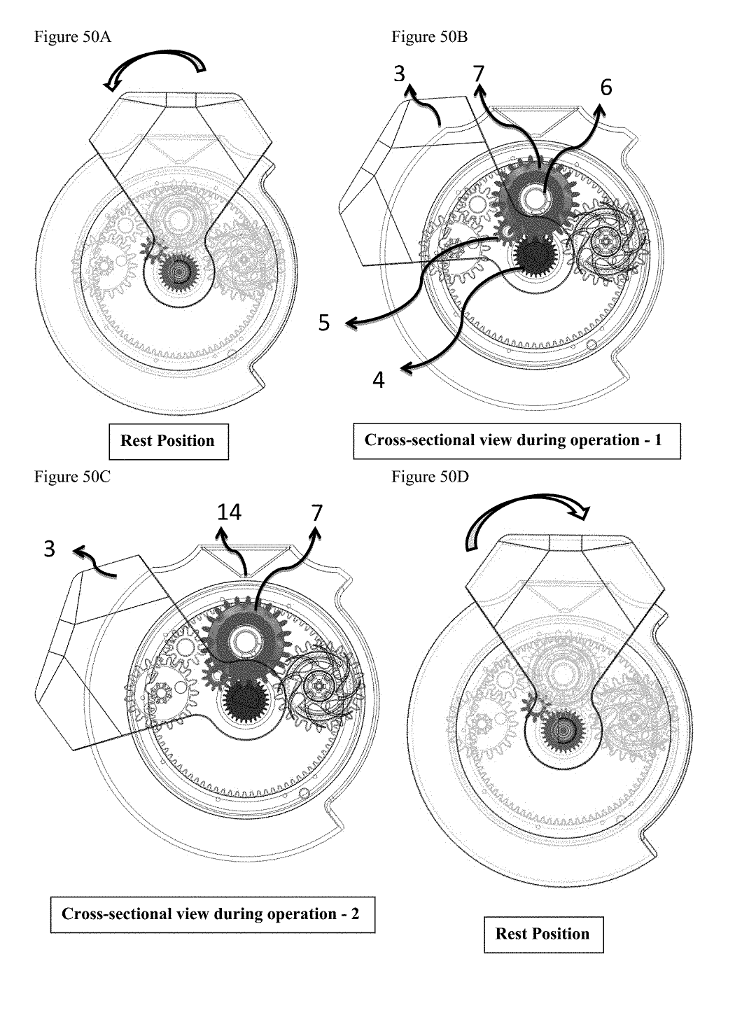

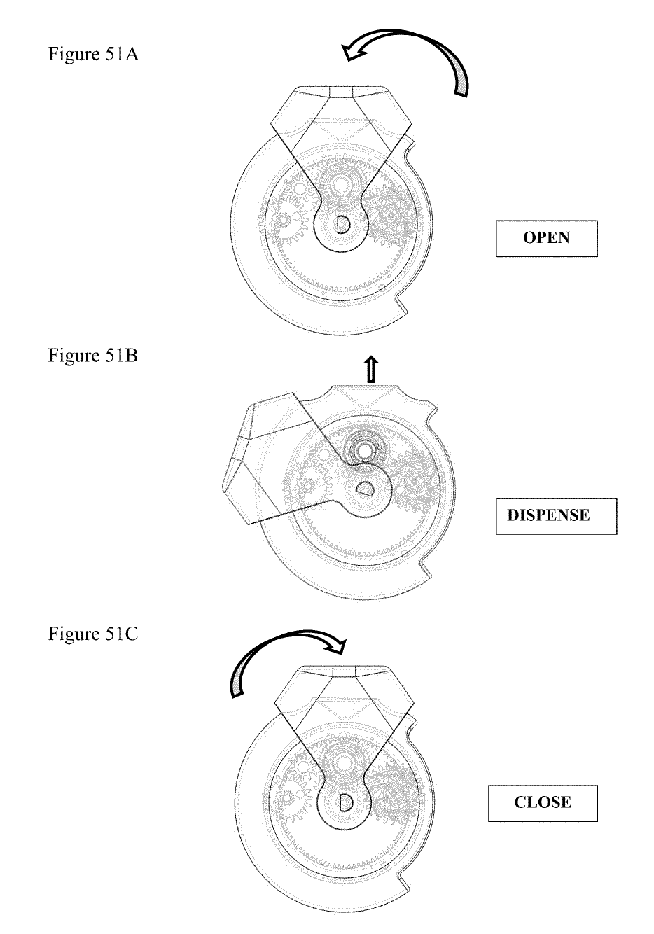

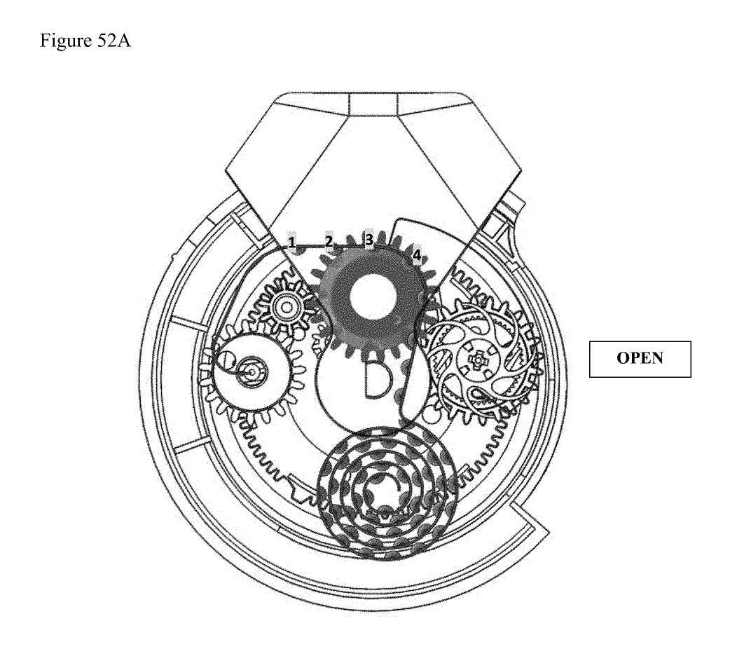

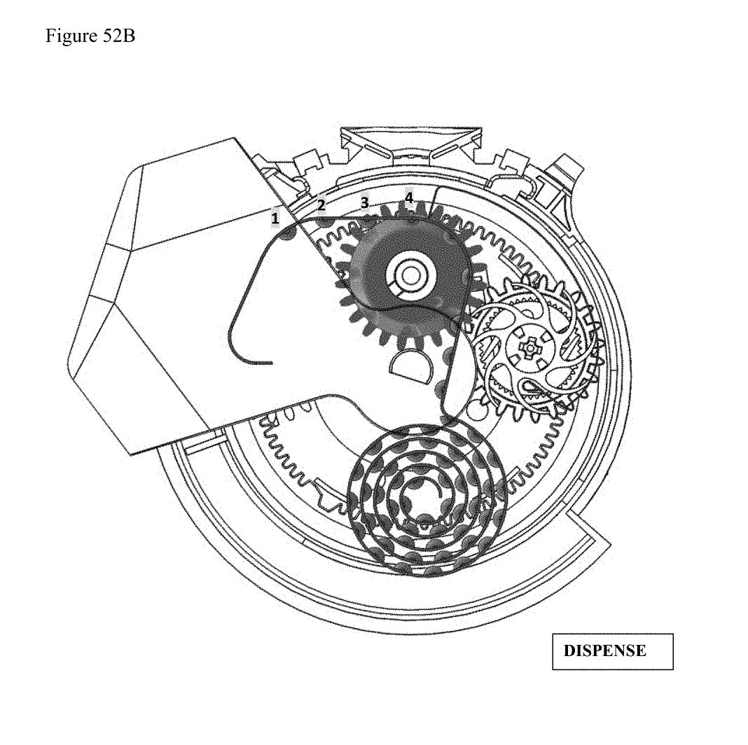

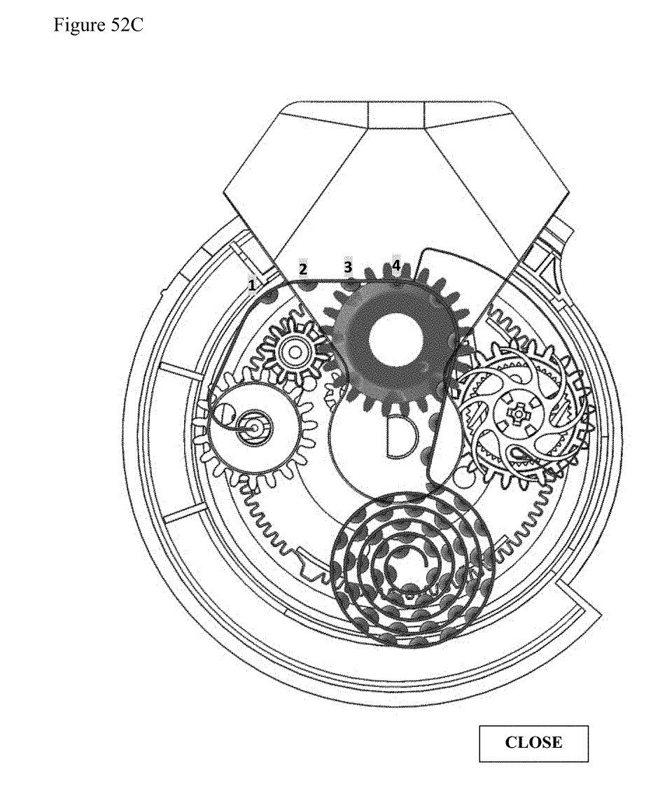

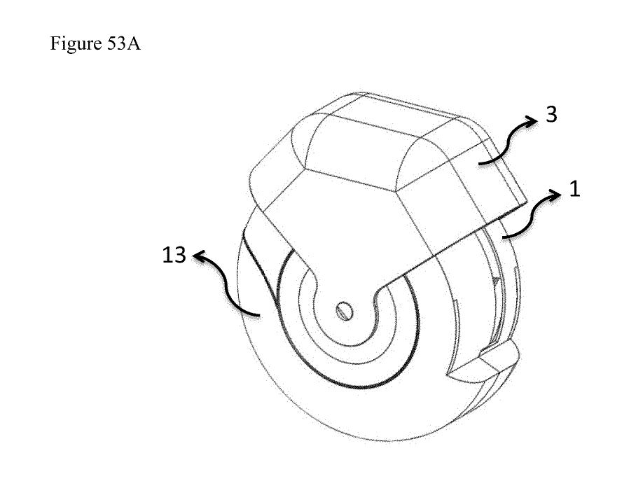

The mechanism of operation of the medicament dispenser is presented in FIGS. 50A-50D and 51A-51C. In the initial (rest) state the outer cover (3) covers the flow channel (14) of the diskette assembly. The inhaler assembly can include a diskette assembly, central gear and an outer cover. The blister advancing mechanism is located within the diskette assembly. The components of the diskette assembly (1) are the central gear (4), cuvette ratchet (6), blister cuvette (7), winding assembly (11), inner housing (8), dose ring (10), interim gear (12) and wrapping wheel (13). The outer cover (3) and central gear (4) are the primary actuators for the diskette assembly. The central gear (4) rotates about the bearing (21). The teeth (20) on the central gear (4) mate with the teeth (22) of the idle gear (5). The cuvette ratchet (6) is located within the blister cuvette (7) and is responsible for discrete angular movement of the blister cuvette (7). The cuvette ratchet (6) also prevents the reverse angular movement of the blister cuvette (7), hence moving the blister cuvette (7) in one angular direction advancing the blister. The blister cuvette (7) mates with the winding assembly (11) and the wrapping wheel (13) via the interim gear (12). When the outer cover (3) of the medicament inhaler is rotated (opened) uni-directionally, it enables the rotation of the central gear (4) keyed to the outer cover (3). The calculated gear ratio between the central gear (4) and the cuvette ratchet (6) gear rotates the cuvette ratchet (6) and the blister cuvette (7) via the idle gear (5). After completion of the operation (actuation) by the outer cover (3) the blister cuvette (7) is aligned with the flow channel (14). At this stage the contents of the blister pocket are available for dispensing via the flow channel (14). During this operation the lamination winds around the winding assembly (11), the blister pockets advance via the wrapping wheel (13) and the blister is separated from the blister advancing assembly via the inner housing (8). Once the contents of the blister pockets are dispensed the outer cover (3) is rotated (closed or retracted). At this stage outer cover (3) covers the flow channel (14) of the diskette assembly and travels back to home (rest) position.

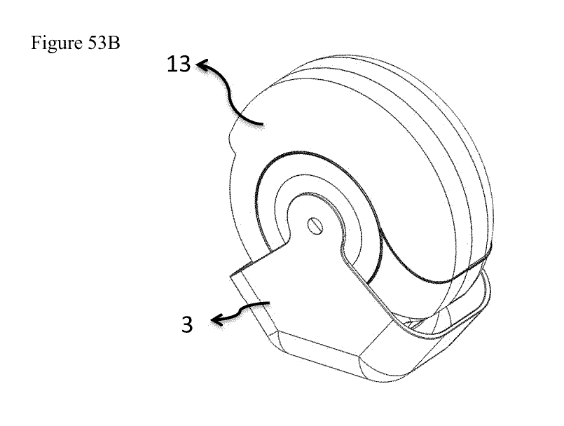

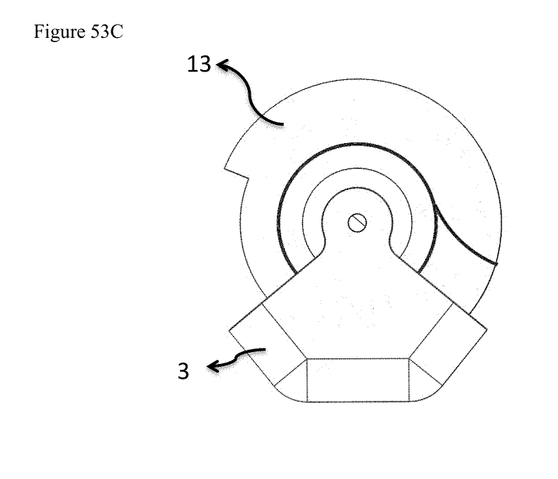

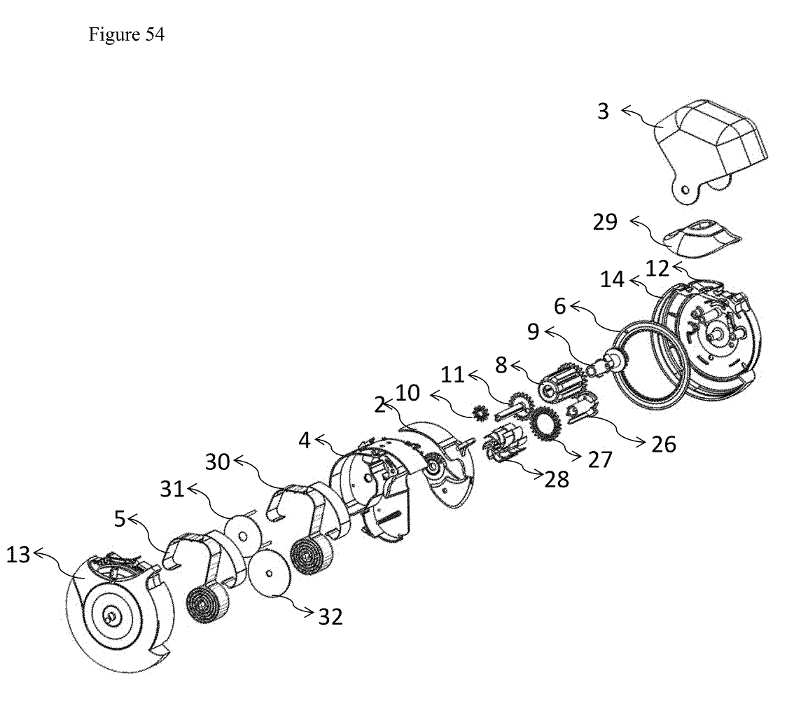

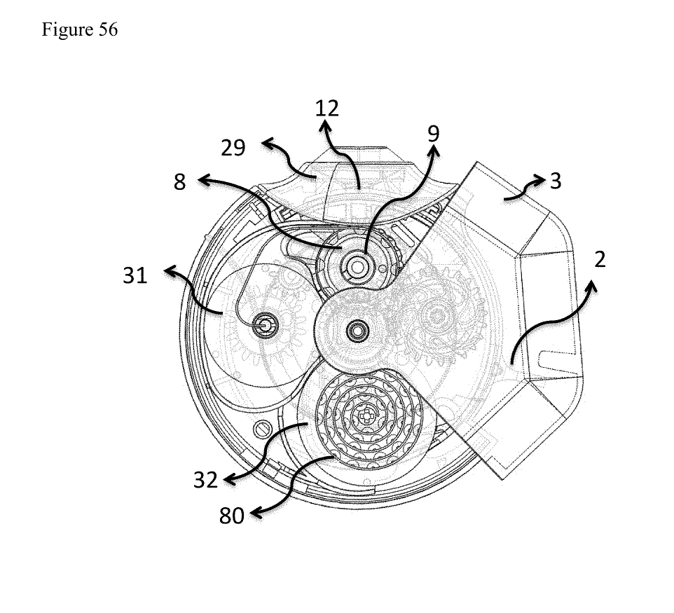

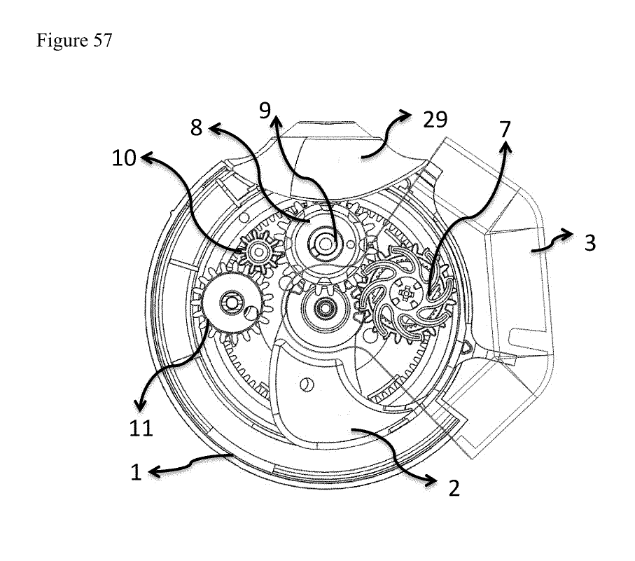

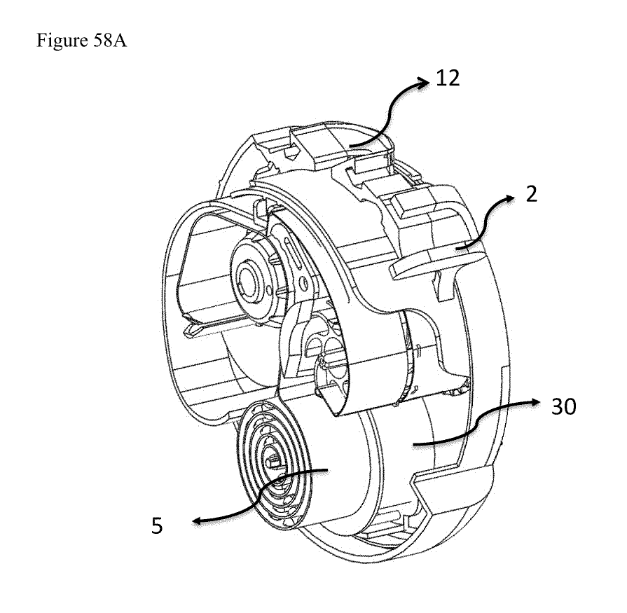

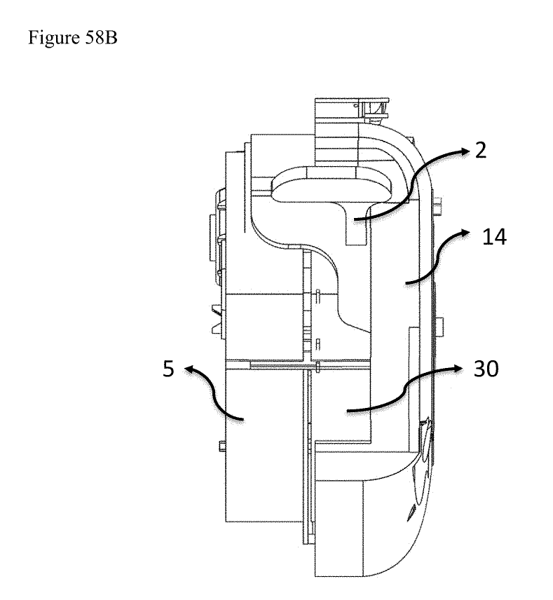

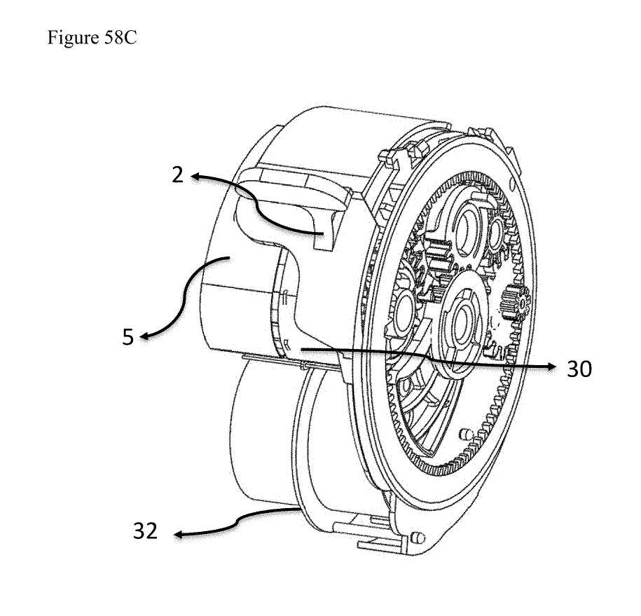

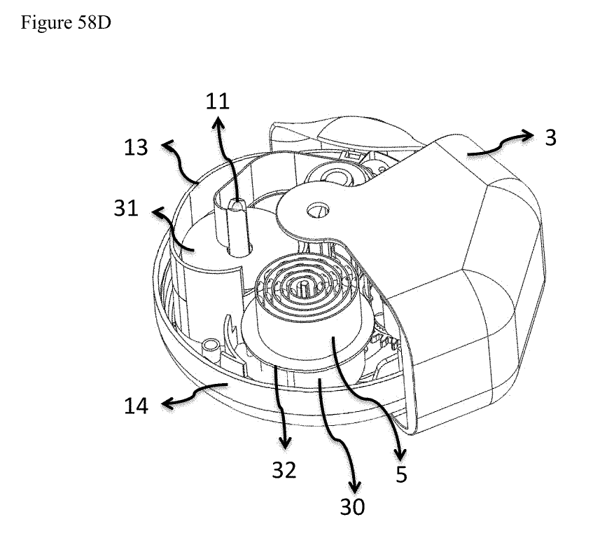

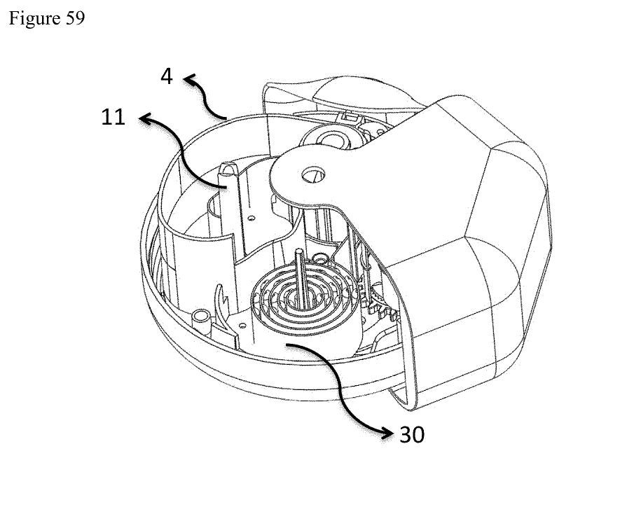

FIGS. 53A-53C illustrate different views of the medicament dispenser according to another aspect of the disclosure. The medicament dispenser can include a diskette assembly (1) which houses two reels of blisters and the blister advancing mechanism. The blister advancing mechanism (see FIGS. 54, 56 to 57) can be operated (or actuated) via the lever (2). The lever (2) can be operated (or actuated) via the outer cover (3).

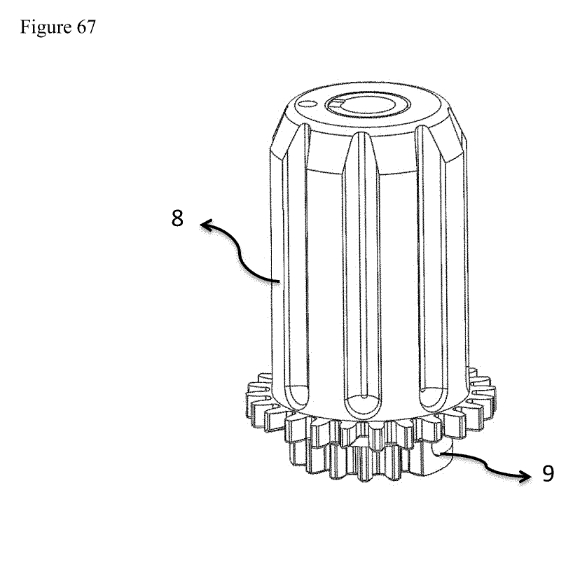

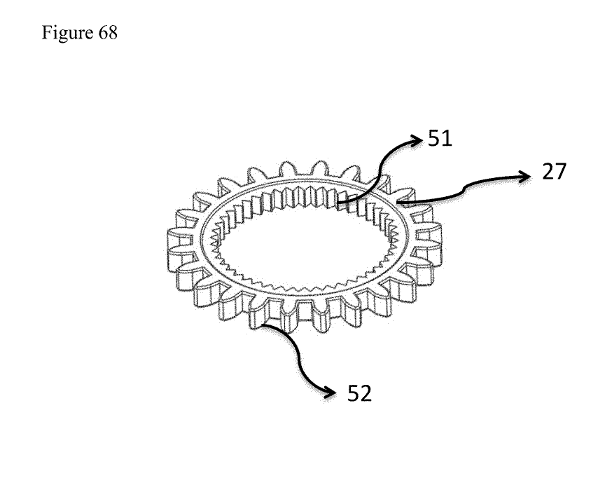

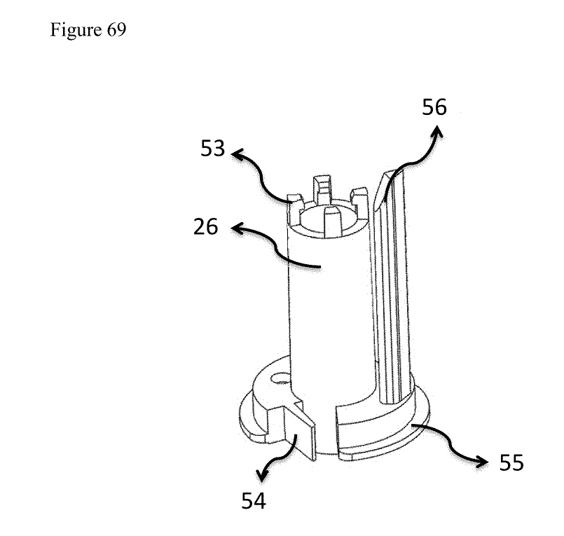

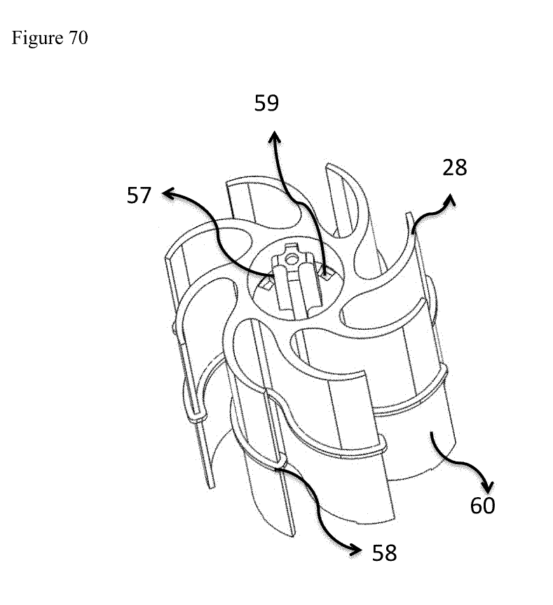

As shown in FIG. 54, the diskette assembly (1) can include the base cover (14) and top cover (13) for enclosing two reels of blisters (5) and (30), inner housing assembly, dose ring (6), deck separators (31) and (32), the blister advancing mechanism and lever (2). The blister advancing mechanism can be protected by a top cover (13). The top cover (13) can be locked with the base cover (14). The blister advancing mechanism components enclosed within the diskette assembly can be the cuvette ratchet (9), blister cuvette (8), winding assembly (7), inner housing assembly (4), interim gear (10) and wrapping wheel (11). The operation (or actuation) of lever can rotate the cuvette ratchet (9) by a discrete angular movement via the spur gears (24). The cuvette ratchet (9) is located within the blister cuvette (8) (see FIG. 67) and is responsible for discrete angular movement of the blister cuvette (8). The cuvette ratchet (9) can also prevent the reverse angular movement of the blister cuvette (8), hence moving the blister cuvette (8) in a unilateral angular direction advancing the reels of blisters (5) and (30). The blister cuvette (8) can mate with the winding assembly (7) and the wrapping wheel (11) via the interim gear (10).

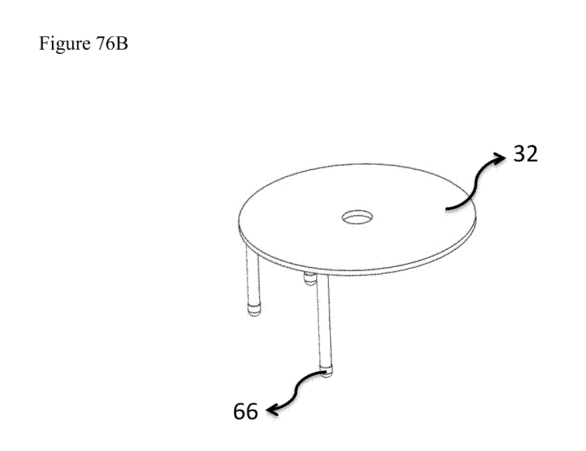

The two reels of blisters (5) and (30) can include a lid sheet (81), and a base sheet (82) containing individual blister pockets (80) having powder, tablet, capsule, pellet or puck to be delivered by the medicament dispenser. The two reels of blisters can be separated in two decks within the diskette assembly by deck separators (31) and (32) located in the inner housing assembly (4) (see FIGS. 54, 74-76B, 79A-79B). The blister cuvette (8) can rotate synchronously and unidirectional with the cuvette ratchet (9), thereby advancing the blister pockets (80) from the two reels of blisters (5) and (30). The two reels of blisters (5) and (30) that incorporate the blister pockets (80) are laminated at the other end.

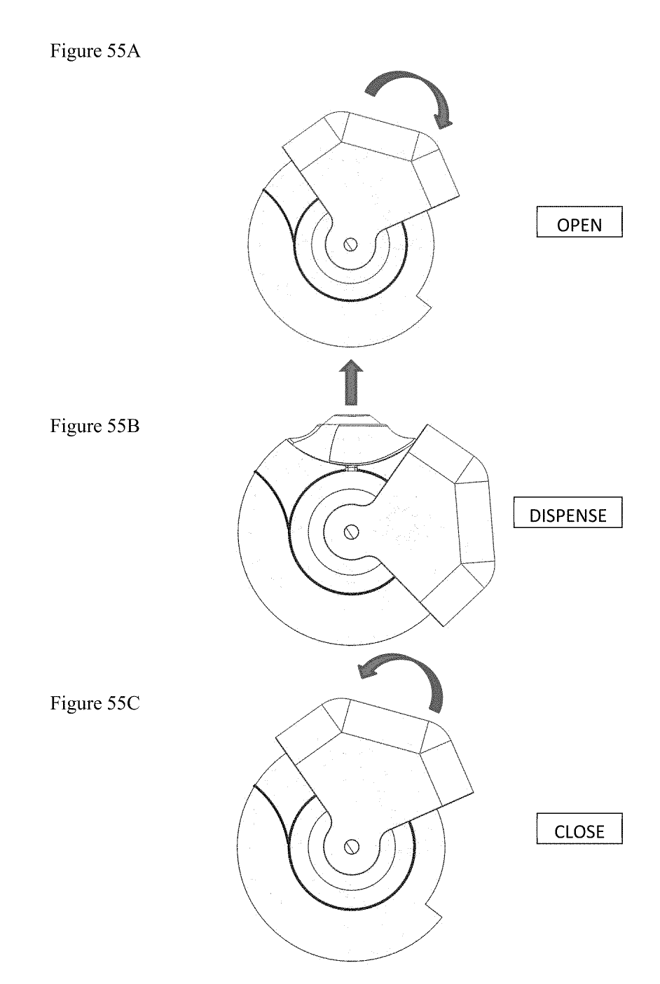

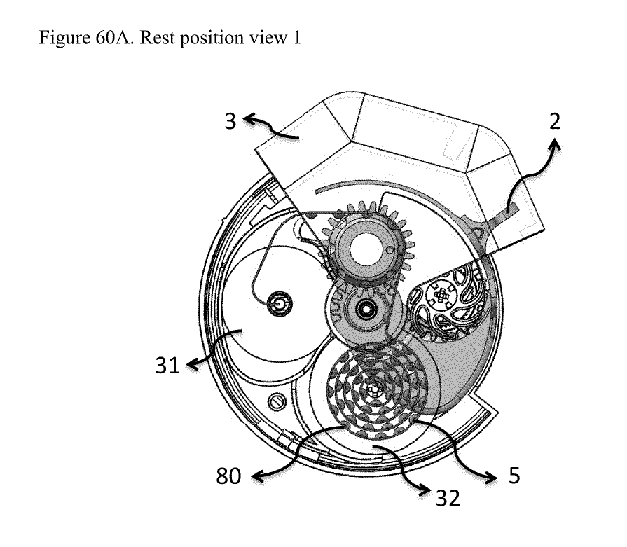

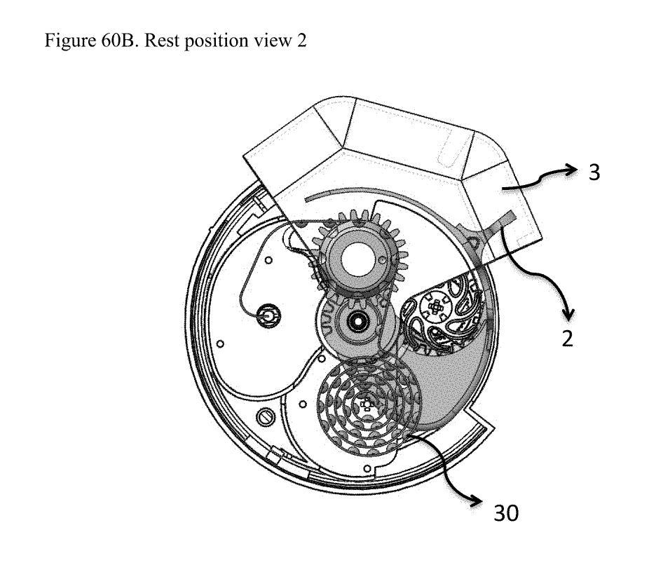

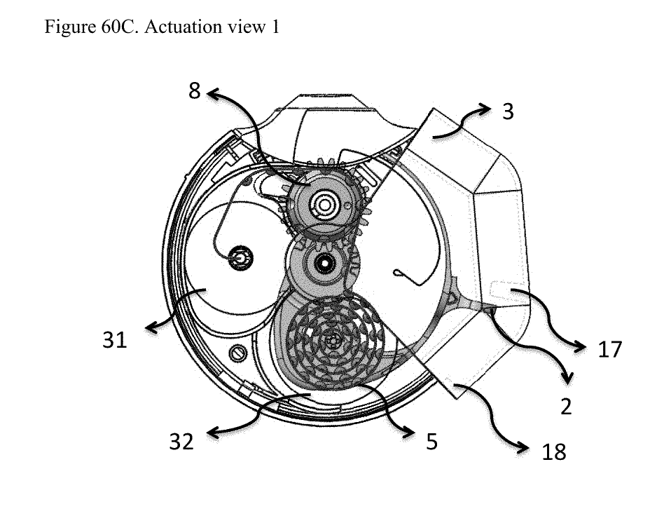

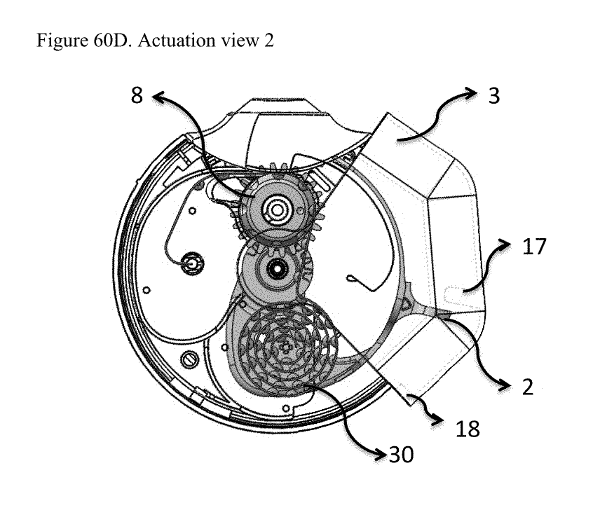

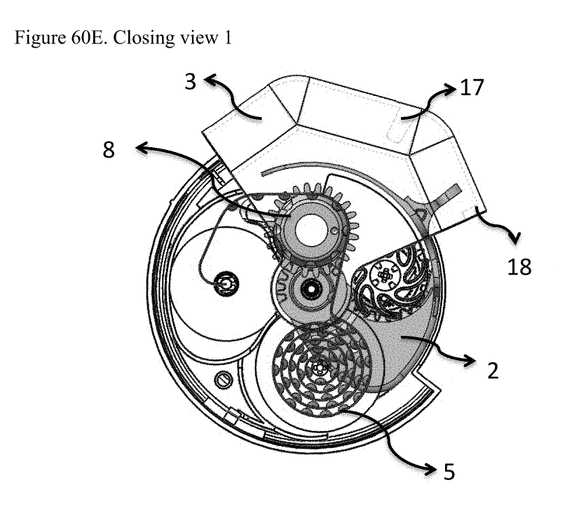

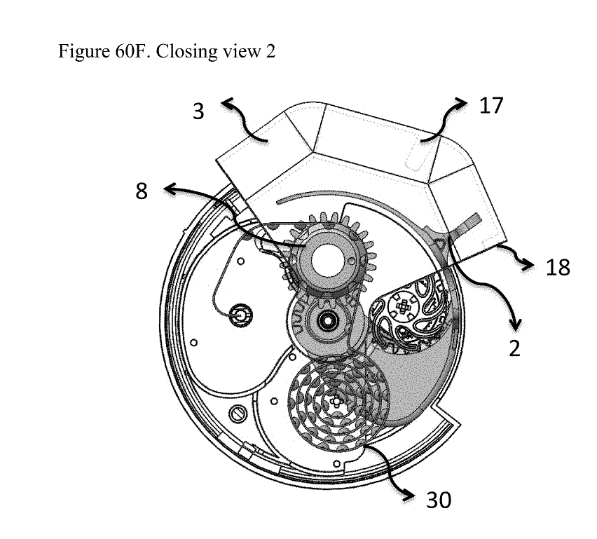

During operation of the medicament dispenser (FIGS. 55, 60A-60F) the two blister pockets (80) can simultaneously align with the flow channel (opening) (12) of the diskette assembly. The laminations of the two reels of blisters can be simultaneously peeled during movement of each reel of blisters via a constant tension torque applied by the winding assembly (7).

During operation of the medicament dispenser (see FIGS. 60A-60F), the outer cover (3) can be rotated to expose the flow channel (opening) (12). The motion of the outer cover (3) can rotate the cuvette ratchet (9) via the lever (2) thereby advancing the reels of blisters (5) and (30).

The lid sheets (81) from the two reels of blisters can wind around the winding assembly (7). By the same mechanism the base sheets (82) having blister pockets (80) can be advanced via the wrapping wheel (11). The wrapping wheel (11) rotates in the opposite direction of the blister cuvette (8) via the interim gear (10). The two reels of blisters (5) and (30) can be separated from the blister advancing mechanism via an inner housing assembly (4). Each reel of blister can also be separated by deck separators (31) and (32) located on the inner housing assembly (4). The blister advancing mechanism is protected by a top cover (13). The top cover (13) can be locked with the base cover (14) and can be permanently attached to the base cover (14) through welding techniques. The outer cover (3) pivots on the diskette assembly which provides for the final assembly of the medicament dispenser.

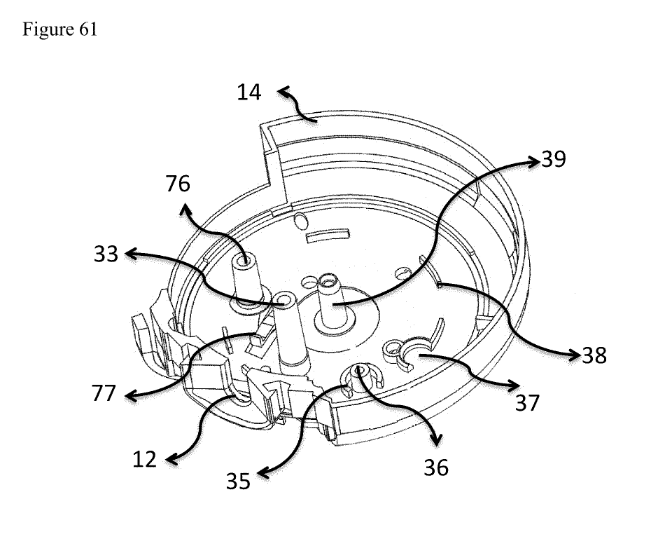

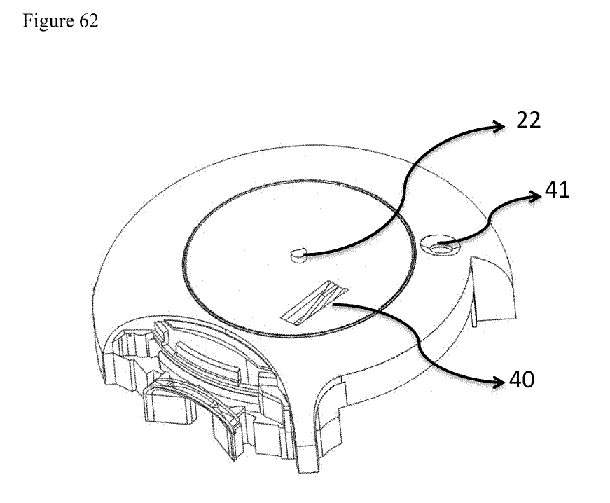

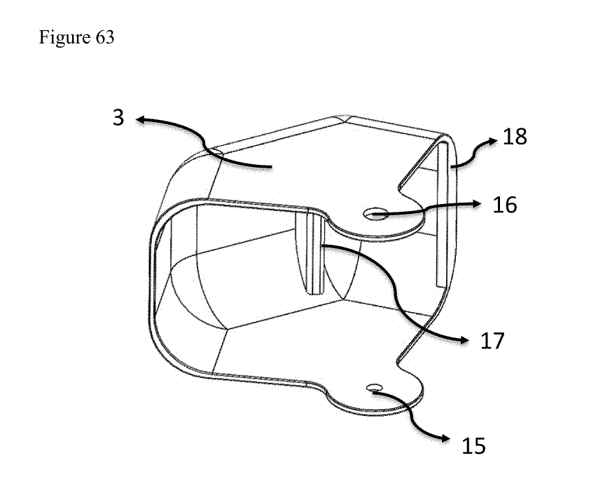

The base cover (14) (FIGS. 54, 61-62) can hold and encase the components of the blister advancing mechanism. The lever (2) can rotate around the center (20). The winder boss (76) locates the winding assembly (7) that rotates about it. The cuvette ratchet (9) can rotate about the anterior boss (33). Stud (36) locates the interim gear (10) and rests on the platform (35). Curved ridge (37) locates the wrapping wheel (11). Circumferential ribs (38) locate and guide the dose ring (6). Spring (40) engages with the blister cuvette (8) via the locking surface (77) and provides an auditory feedback in the form of the sound of a click when the mechanism is actuated and is ready for the user to intake the dose. Window (41) provides a view of the dose ring (6) for user feedback on number of remaining doses.

The mouthpiece (29) (FIG. 54) can allow the user to receive the contents of the reel of blisters (5) and (30) by placing the mouthpiece interface opening inside the oral cavity. The mouthpiece interface opening allows the contents of the reels of blisters (5) and (30) to pass through from the flow channel (12) of the diskette assembly. The mouthpiece interface opening is covered by the outer cover (3) when the medicament dispenser is not in use. The mouthpiece (29) can snap on the diskette assembly (1).

The outer cover (3) (FIGS. 53A-57, 60A-60F, 63, 64A-64B) has two main functions. The outer cover (3) covers the mouthpiece (29) and mouthpiece interface opening when the medicament dispenser is not in use. The outer cover (3) actuates the blister advancing mechanism via the lever (2). The first ridge (17) is an actuation ridge on the outer cover (3). The second ridge (18) retracts the lever (2) back to initial state. The first ridge (17) can engage with the lever (2) and pushes the lever (2) at the lever surface (19). As the outer cover (3) is rotated in the clockwise direction, the lever (2) rotates along with the first ridge (17) and actuates the blister advancing mechanism inside the diskette assembly (1). When the outer cover (3) is rotated (closed) in the counter-clockwise direction, the second ridge (8) rotates the lever (2) back to home position via the lever surface (19). The counter-clockwise motion of the outer cover (3) rotates the cuvette ratchet (9) via the lever (2) without advancing the reel of blisters (5) and (30). During the operation (or actuation) of the outer cover (3), a distal gear (67) attached to the wrapping wheel (11) rotates the dose ring (6) to display the number of doses consumed by the user.

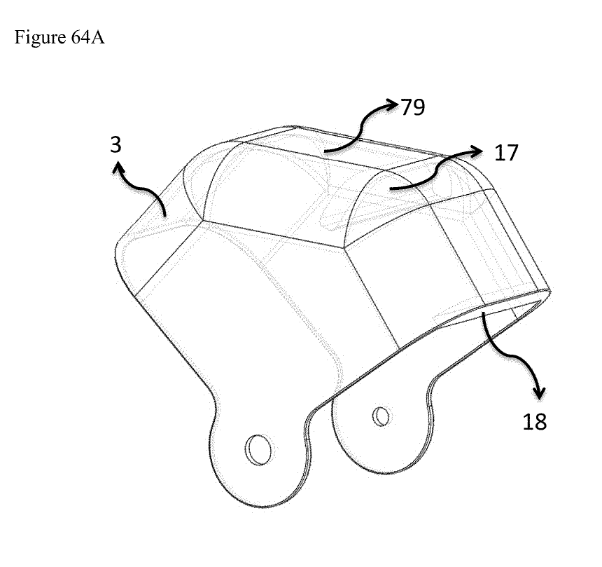

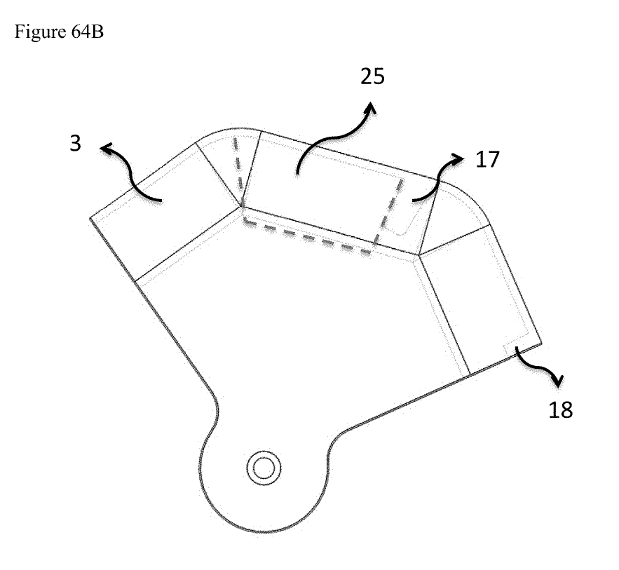

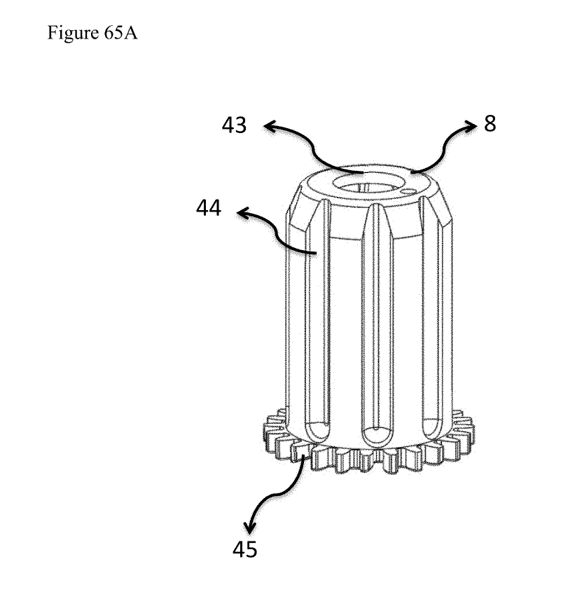

Mounting holes (15) and (16) mate with the studs (22) and (23) on the base cover and top cover, respectively. The outer cover can have a flat top surface (79). The compartment (25) of the outer cover (3) can have a dedicated space to accommodate for a power source and electronic instruments (see FIG. 64B).

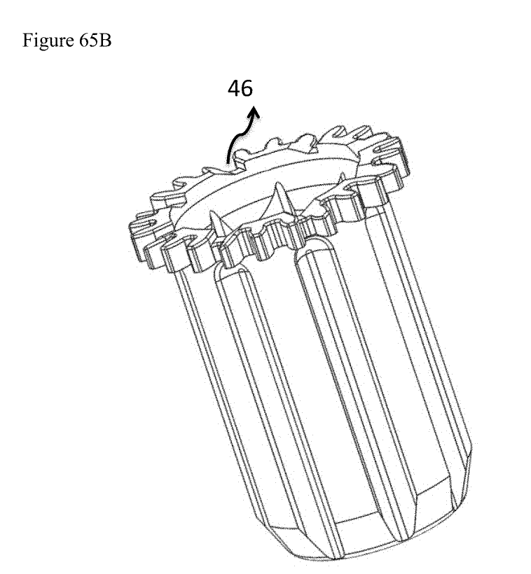

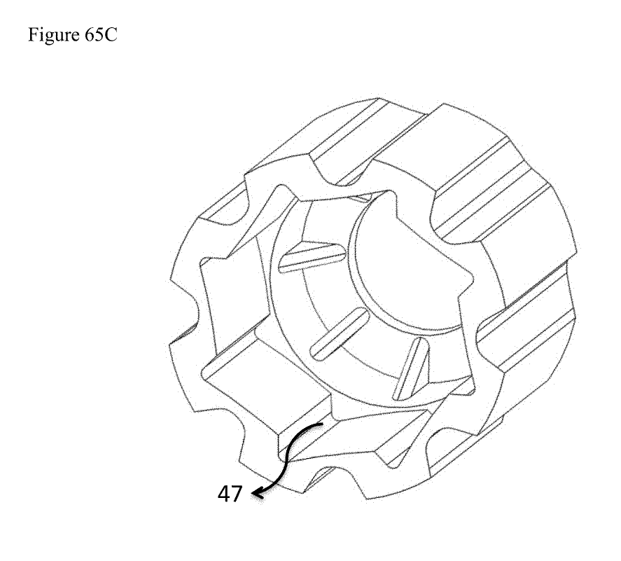

The blister cuvette (8) (FIGS. 54, 65A-65C) can be driven by the ratcheting mechanism of the cuvette ratchet (9). The cross sectional view in FIG. 65C shows the driver surface (47) that can be used to rotate the blister cuvette (8). The guide (43) rides on the cuvette ratchet (9). The cuvettes (44) on the blister cuvette (8) locate the individual blister pockets on the reels of blisters. The gears (45) mate with the interim gear (10) and the winder gear (27). The grooves (46) on the blister cuvette (8) mates with the spring (40) on the base cover (14) to provide an auditory feedback to the user to intake the dose.

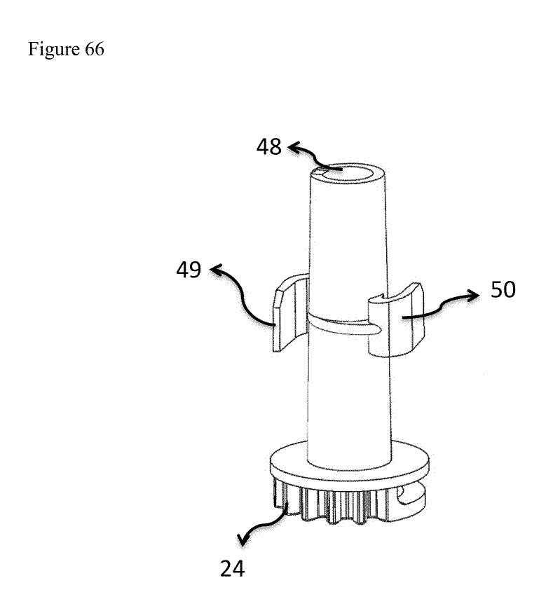

The cuvette ratchet (9) (FIG. 66) rides over the anterior boss (33) of the base cover (14). The cuvette ratchet (9) is guided on the anterior boss (33) by the bearing (48). The cuvette ratchet (9) is driven by the lever (2) via the spur gears (24). The ratchets (49) and (50) located diametrically opposite on the cuvette ratchet (9) drive the blister cuvette (8) via the driver surface (47) of the blister cuvette (8). The ratcheting mechanism of the cuvette ratchet (9) prevents the rotation of the blister cuvette (8) in the opposite direction. The ratchets (49) and (50) ride over the inner wall of the blister cuvette (8) without rotating the blister cuvette (8).

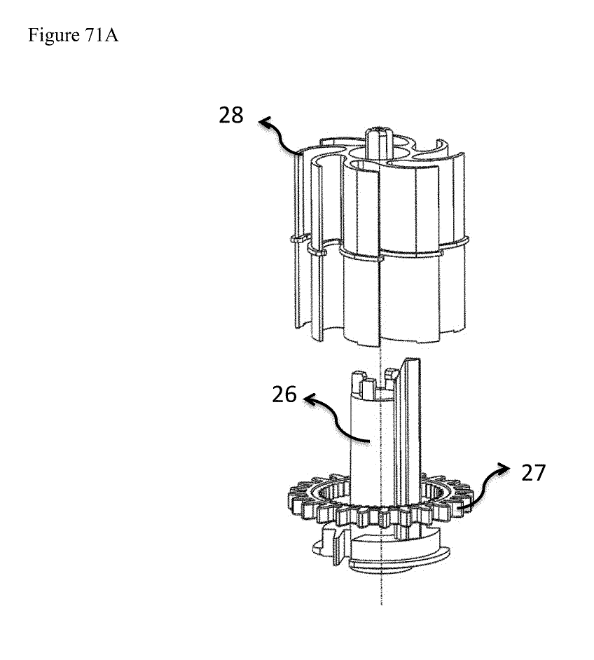

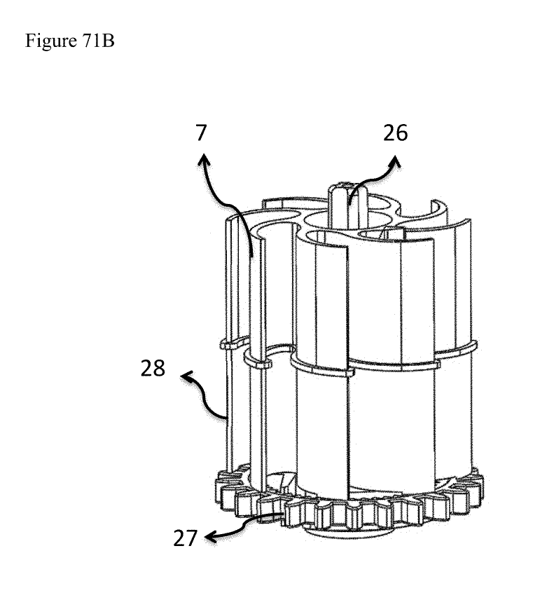

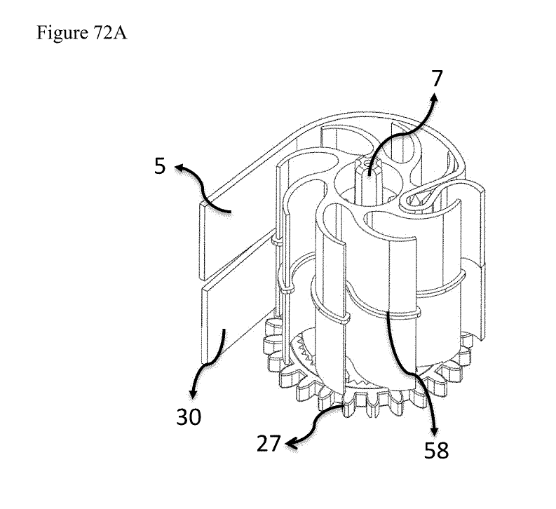

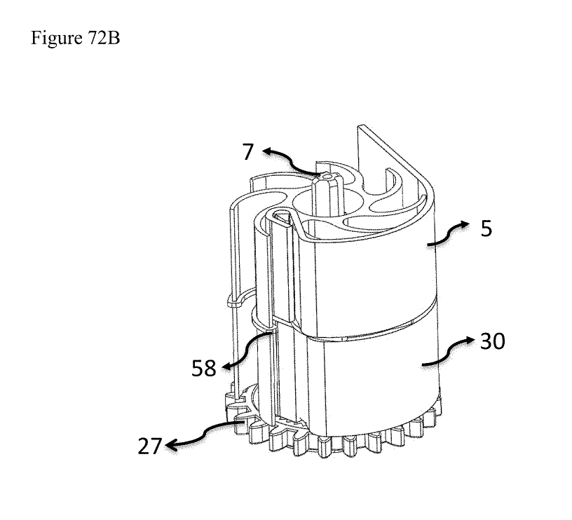

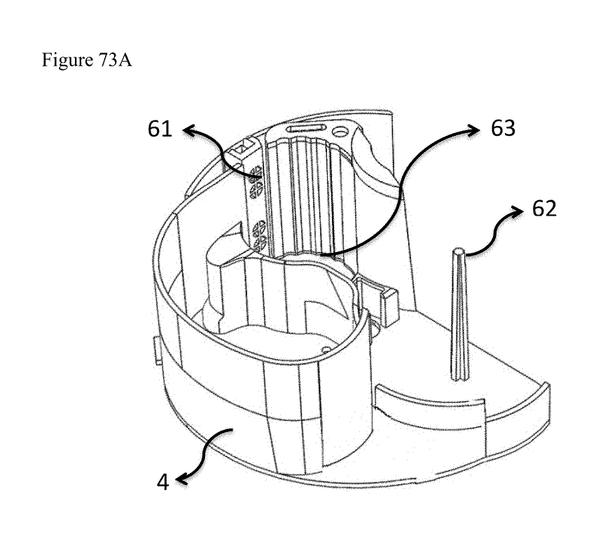

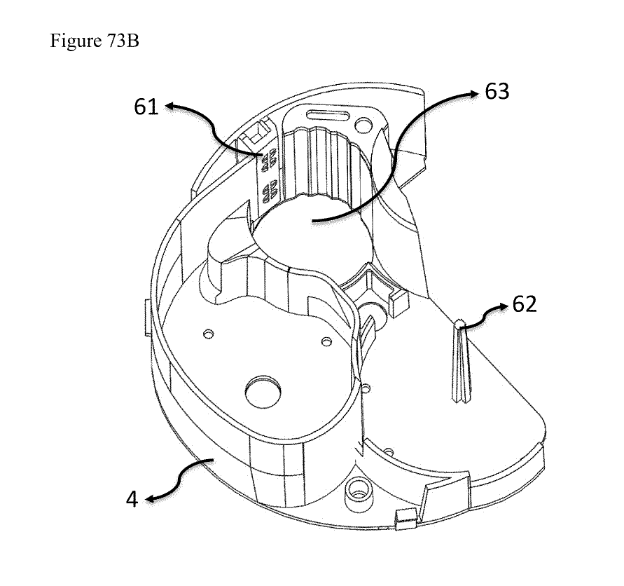

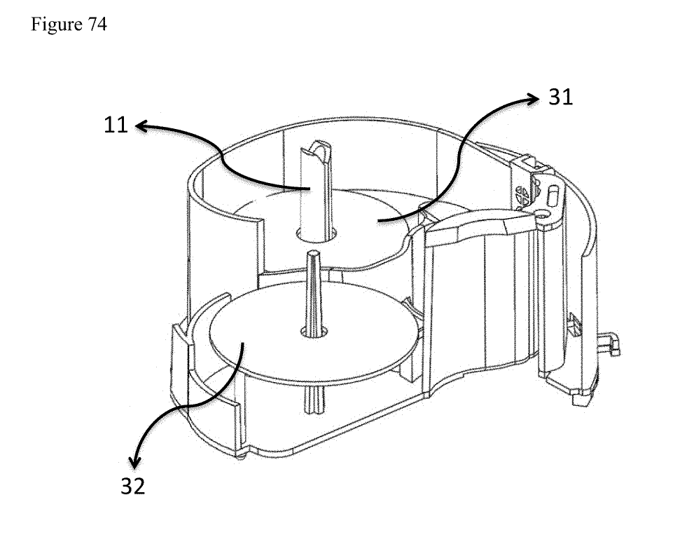

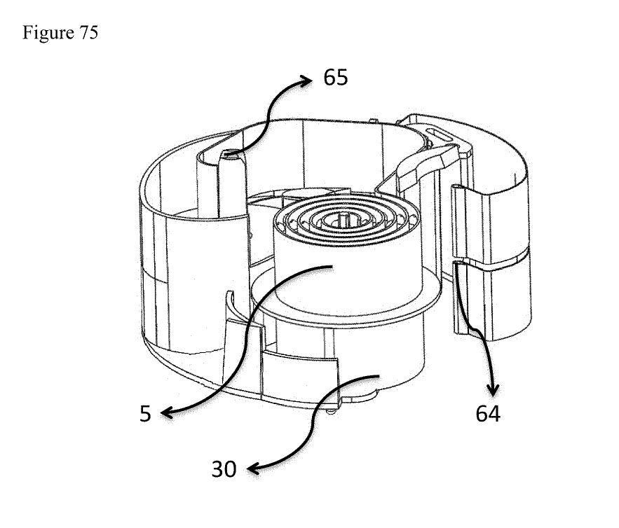

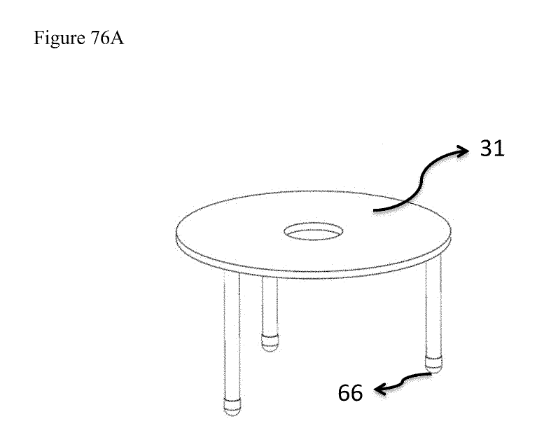

The winding assembly (7) (FIGS. 71A-72B) can include the winder rotor (28), winder gear (27) and the winder ratchet (26). The winder gear (27) seats on the flange (55) of the winder ratchet (26). The internal gears (51) drive the winder ratchet (26) and hence the winder rotor (28). The ratchet (54) on the winder ratchet (26) is engaged with the winder gear (27) and also prevents it rotation in the opposite direction. The winder gear is driven by external gears (52) that mate with the gears (45) on the blister cuvette (8). The locking posts (53) on the winder ratchet (26) lock with the winder rotor (28) through the recess opening (59). Winding Post (57) is used to apply initial tension to the reel of blisters (5) and (30) during the assembly process. The blister post (56) is located within the closed loop formed on the reels of blisters (5) and (30) at one end. As winding assembly (7) rotates, the laminations of reels of blisters (5) and (30) can be wrapped and wound around the winding surface (60). Ridge (58) on the winder rotor (28) separates the reel of blisters (5) and (30) during the winding process. FIGS. 71A-72B illustrate the exploded and assembled views of the winding assembly.

The inner housing assembly (4) (FIGS. 73A-73B) separates the medicament carrier advancing mechanism from the reels of blisters (5) and (30). The inner housing assembly (4) houses two reel of blisters separated in two decks via the deck separators (31) and (32). Deck separators (31 and 32) allow for independent movement of two reels of blisters with no contact between reel of blisters and no friction between reels of blisters due to mutual contact. Mesh (61) allows for the de-agglomeration of the contents of the reels of blisters (5) and (30). During dosing operation, the cuvette (44) on the blister cuvette (8) is aligned with the mesh (61). The reels of blisters (5) and (30) are wound around the post (62) and can be separated by deck separator (32). One end of the reel of blister has a sealed loop (64) that is placed in the blister post (56) of the winder ratchet (26). The other end (83) of the reel of blister (5) is placed in the slit (65) of the wrapping wheel (11). The assembly of the blister cuvette (8) and the cuvette ratchet (9) is located in cavity (63). The deck separators (31) and (32) lock in the inner housing assembly (4) by the locking head (66) on each of the deck separators (31) and (32).

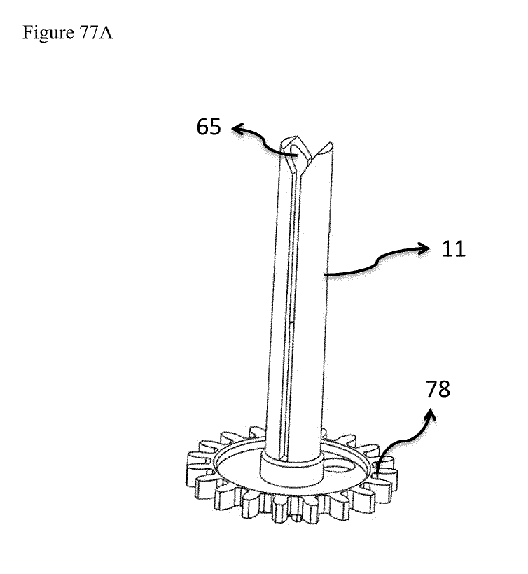

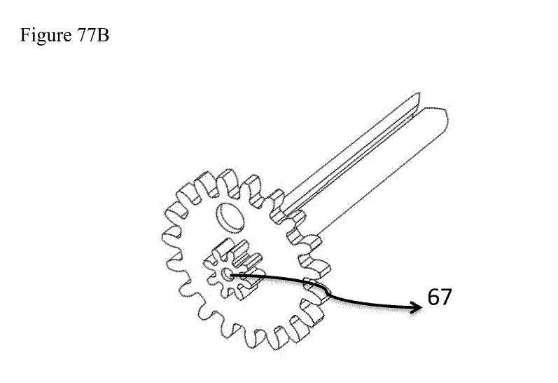

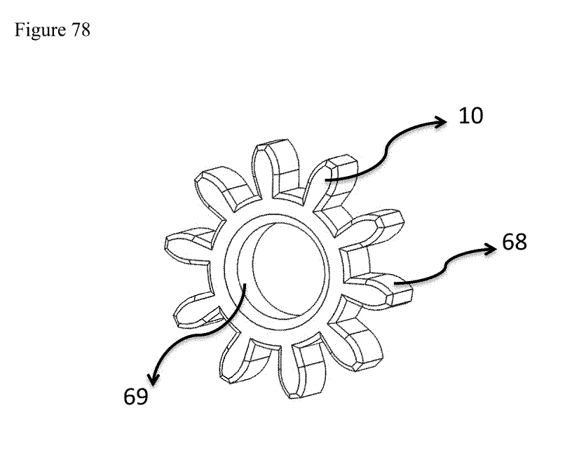

The wrapping wheel (11) (FIGS. 77A-77B) is located within the curved ridge (37) of the base cover (14). The wrapping wheel (11) wraps one part of the reels of blisters (5) and (30). The end of both the reel of blisters (5) and (30) is secured in the slit (65) of the wrapping wheel (11). The two reels of blister strip (5) are separated by the deck separator (31) and secured in the slit (65). The wrapping wheel (11) is driven by the interim gear (10). The gears (78) of the wrapping wheel (11) mate with the gears (68) of the interim gear (10). Distal gears (67) on the wrapping wheel (11) drive the dose ring (6). Stud (36) locates the interim gear (10) on the bearing (69) of the interim gear (10).

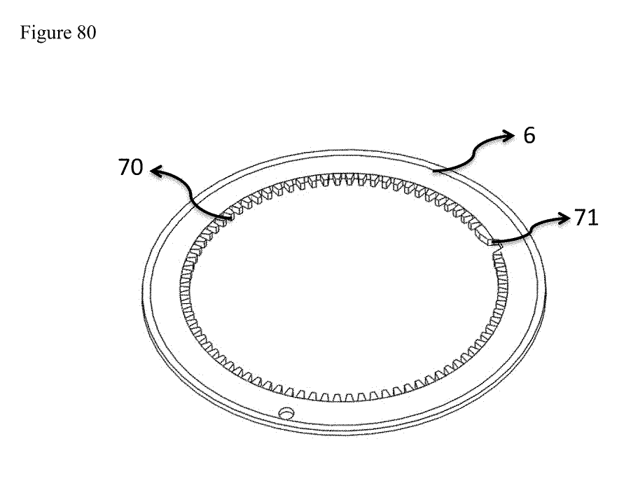

The dose ring (6) (FIGS. 54 and 80) provides feedback to the user of the number of doses remaining while using the device. The dose ring (6) is driven by distal gears (67) of the wrapping wheel (11). Internal gears (70) mate with the distal gears (67) of the wrapping wheel (11). Slot (71) on the indicator wheel (6) allows disengagement of the indicator wheel (6) with distal gears (67) of the wrapping wheel (11). This prevents the rotation of the dose ring (6) once it reaches the final dose.

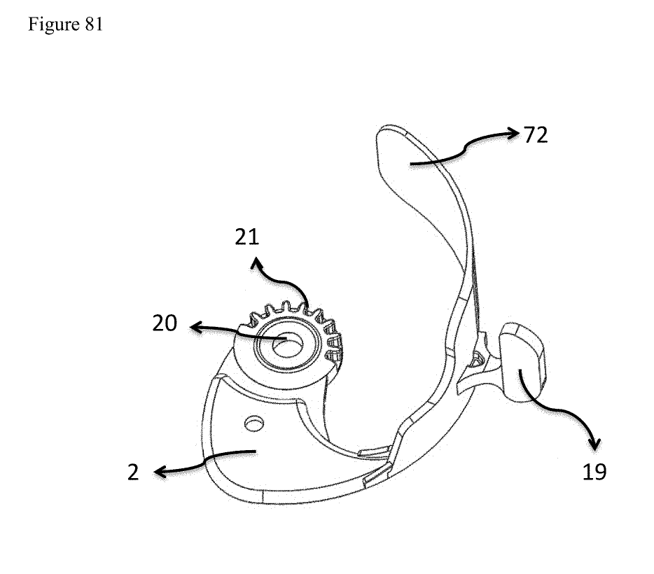

The lever (2) (FIG. 81) is located within the central boss (39) of the base cover (14) via the center (20). The lever (2) rotates about the center (20). The lever (2) can include a lever surface (19) that provides a contact surface for the first ridge (17) of the outer cover (3). Lever teeth (21) mate with spur gears (24) of the cuvette ratchet (9). Lever flap (72) uncovers and covers the mesh (61) of the inner housing assembly (4) during actuation and release. As the lever (2) rotates nominally 20°, the blister advancing mechanism inside the diskette assembly (14) is rotated via the lever teeth.

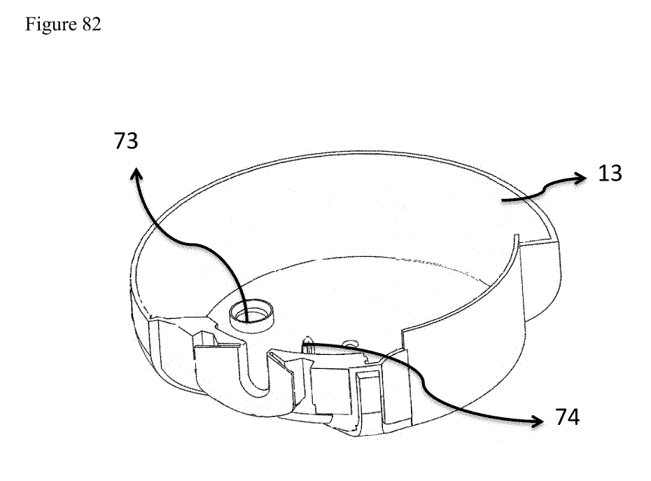

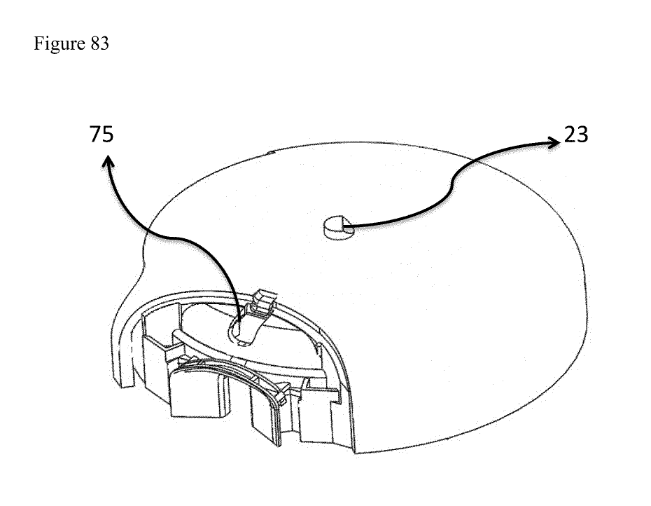

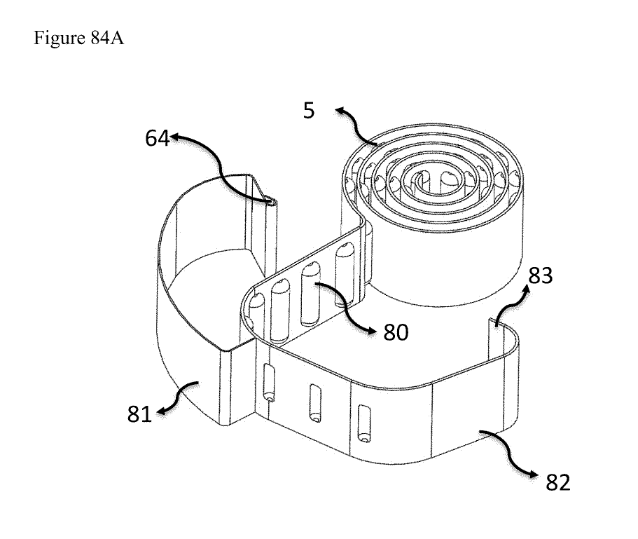

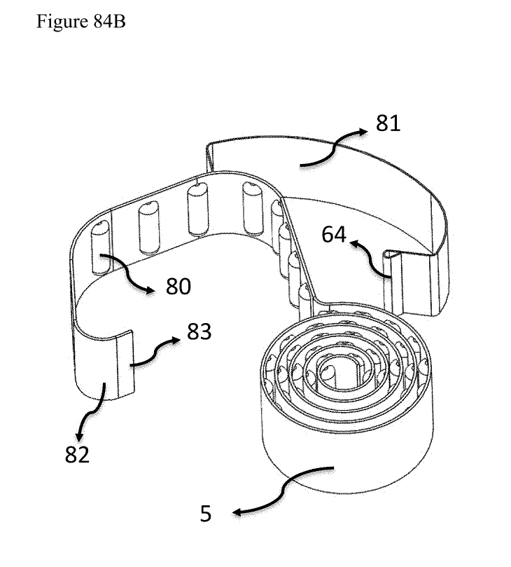

The top cover (13) (FIGS. 82 and 83) encloses the complete medicament carrier advancing mechanism and the reel of blisters (5) and (30). The hollow boss (73) supports the wrapping wheel (11). The mouthpiece interface opening and flow channel (12) allow the contents of the reels of blisters (5) and (30) to be dispensed to the user. Post (74) supports the cuvette ratchet (9). Vent (75) allows passage for air into the device during dosing maneuver. The outer cover (3) rotates about the studs (22) and (23) via the mounting holes (15) and (16) on the outer cover (3).

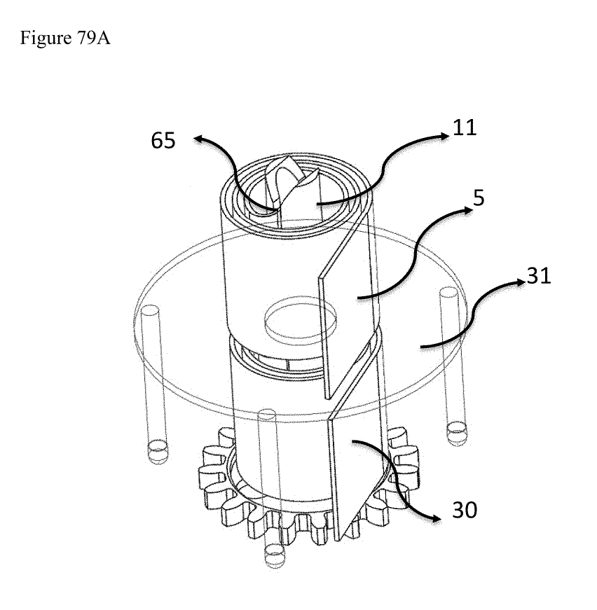

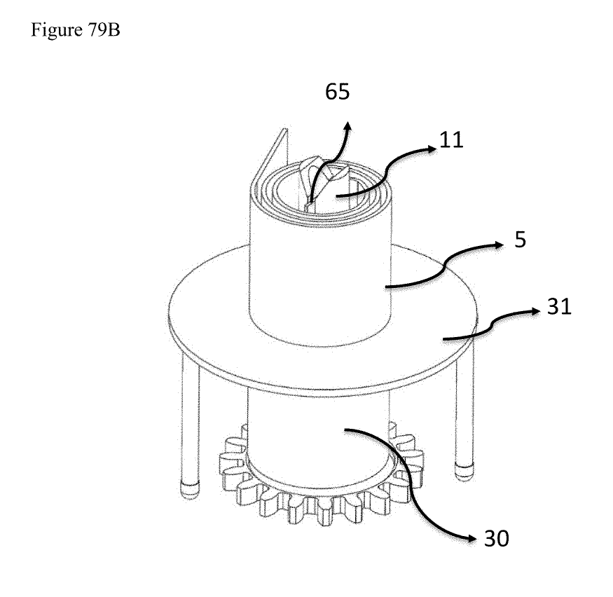

The mechanism of operation of the medicament dispenser is presented in FIGS. 55A-55C and 60A-60F. In the initial (rest) state the outer cover (3) can cover the flow channel (12), mouthpiece (29) and the mouthpiece interface opening of the diskette assembly (1) (FIG. 55A). When the outer cover (3) of the medicament dispenser is rotated clockwise (opened) the outer cover first ridge (17) on the outer cover comes in contact with the lever (2) on the lever surface (19). The lever (2) is in the rest state before outer cover is operated. As the outer cover (3) is rotated, the lever (2) rotates along with the lever surface (19) and actuates the blister advancing mechanism inside the diskette assembly (1) (FIG. 55B). The actuation of lever rotates the cuvette ratchet (9) by a discrete angular movement via the spur gears (24). The blister cuvette (8) rotates synchronously and uni-directionally with the cuvette ratchet (9), thereby advancing the blister pockets (80) from the two reels of blisters (5) and (30). At this stage the two blister pockets (80), one from each reel of blister, align with the flow channel (opening) (12) of the diskette assembly. The lamination of the blister pockets (80) is peeled via a constant tension torque applied by the winding assembly (7) and contents of the blister pockets (80) are available for dispensing via the mouthpiece interface opening and the flow channel (opening) (12). During the actuation and dispensing phase the two reels of blisters (5) and (30) independently rotate and move from the rest position in a unidirectional and synchronous movement. The two reels of blisters glide on the deck separators (31) and (32) without coming in contact with each other and without any friction due to contact between the two reels of blisters (5) and (30). Once the contents of the blister pockets are dispensed the outer cover (3) is rotated (closed) in the counter-clockwise direction. At this stage the second ridge (18) retracts the lever (2) back to initial state. Once the lever (2) reaches initial state, the second ridge (18) prevents further rotation of the outer cover (3) past the initial state of the lever (2). At this stage outer cover (3) can cover the flow channel (12), mouthpiece interface opening and mouthpiece (29) (FIG. 55C). The lamination winds around the winding assembly (7) (FIGS. 72A-72B). The two separate delaminated base sheets from the two reels of blisters (5) and (30) are coiled independently in a unidirectional, and synchronous movement and remain separated from each other by the inner housing assembly (4) by deck separator (31) (FIGS. 79A-79B).

1. A medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the outer cover is adapted to couple with the lever and the medicament carrier advancing mechanism.

2. The medicament dispenser according to claim 1, wherein the outer cover being movably mounted to the dispenser for sequential movement from an initial position, in which the flow channel is covered, to a dispense position, in which the flow channel is uncovered.

3. The medicament dispenser according to claim 2, wherein the movement of the outer cover from the initial position rotates the lever and actuate the medicament carrier advancing mechanism.

4. The medicament dispenser according to claim 2, wherein the movement of the outer cover from the initial position results in actuation of the medicament carrier advancing mechanism.

5. The medicament dispenser according to claim 1, wherein the outer cover further comprises a first ridge and a second ridge.

6. The medicament dispenser according to claim 5, wherein the first ridge rotates the lever from the initial position of the outer cover to the dispense position of the outer cover to actuate the medicament carrier advancing mechanism.

7. The medicament dispenser according to claim 1, wherein the outer cover further comprises a plunger.

8. The medicament dispenser according to claim 7, wherein the plunger rotates the lever from the initial position of the outer cover to the dispense position of the outer cover to actuate the medicament carrier advancing mechanism.

9. The medicament dispenser according to claim 1, further comprises an inner cover.

10. The medicament dispenser according to claim 9, wherein the outer cover is adapted to couple with the lever and the medicament carrier advancing mechanism via the inner cover.

11. The medicament dispenser according to claim 10, wherein the outer cover further comprises a first ridge and a second ridge.

12. The medicament dispenser according to claim 11, wherein the first ridge rotates the inner cover thereby the lever from the initial position of the outer cover to the dispense position of the outer cover to actuate the medicament carrier advancing mechanism.

13. The medicament dispenser according to claim 1, wherein the actuation of the medicament carrier advancing mechanism rotates a cuvette ratchet and a blister cuvette synchronously and thereby advances the medicament carrier to align with the flow channel for dispensing the medicament.

14. The medicament dispenser according to claim 5, wherein the second ridge retracts the lever back from the dispense position of the outer cover to the initial position of the outer cover.

15. The medicament dispenser according to claim 7, wherein the plunger retracts the lever back from the dispense position of the outer cover to the initial position of the outer cover.

16. The medicament dispenser according to claim 11, wherein the second ridge retracts the inner cover thereby the lever back from the dispense position of the outer cover to the initial position of the outer cover.

17. The medicament dispenser according to claim 1, wherein the medicament dispenser comprises a plurality of said medicament carriers each carrying multiple distinct medicament dose portions.

18. The medicament dispenser according to claim 17, wherein the plurality of the medicament carriers comprises of a first medicament carrier and a second medicament carrier, each of the medicament carriers comprises medicament.

19. The medicament dispenser according to claim 18, wherein the first medicament carrier and the second medicament carrier are separated by a deck separator.

20. The medicament dispenser according to claim 1, wherein the medicament comprises one or more active pharmaceutical ingredients and optionally, one or more pharmaceutically acceptable carriers.

21. The medicament dispenser according to claim 20, wherein the active pharmaceutical ingredient is selected from group consisting of anticholinergics, anti-inflammatories, analgesics, anginal preparations, anti-allergic, antihistamines, antitussives, bronchodilators, anti-infectives, leukotriene inhibitors, PDE IV inhibitors, diuretics, hormones, cromolyns, xanthines, therapeutic proteins and peptides, vaccines, diagnostics and gene therapies or combinations thereof.

22. The medicament dispenser according to claim 18, wherein the first medicament carrier comprises bronchodilator as active pharmaceutical ingredient and the second medicament carrier comprises anti-inflammatory as active pharmaceutical ingredient.

23. The medicament dispenser according to claim 1, wherein the medicament is in the form selected from group consisting of powder, tablet, capsule, pellet or puck, or mixtures thereof.

24. The medicament dispenser according to claim 1, wherein the dispenser is used for dispensing medicament for the treatment of asthma, chronic obstructive pulmonary disease (COPD), bronchitis or chest infection.

25. The medicament dispenser according to claim 1, wherein the dispenser further comprises a dose counting or an indicating mechanism for counting or indicating number of doses released from the dispenser.

26. The medicament dispenser according to claim 25, wherein the dose counter is in the form of mechanical or non-mechanical.

27. The medicament dispenser according to claim 1, wherein the dispenser further comprises a resting surface.

28. The medicament dispenser according to claim 1, wherein the dispenser further comprises a power source, an electronic circuit and an electronic measurement instrument.

29. The medicament dispenser according to claim 1, wherein the dispenser is an inhalation device.

30. A medicament dispenser for dispensing medicament comprising: a) a diskette assembly comprising: i) at least one medicament carrier carrying multiple distinct medicament portions; ii) a medicament carrier advancing mechanism for actuating the distinct medicament portions; iii) a flow channel; iv) a lever; and b) an outer cover for said flow channel, wherein the flow channel is covered by the outer cover in an initial position, and fully revealed when the outer cover is in a dispense position.

31. The medicament dispenser according to claim 30, wherein the outer cover is adapted to couple with the lever and the medicament carrier advancing mechanism.

32. The medicament dispenser according to claim 30, wherein the dispensing of the medicament provides continuous movement of the outer cover from the initial position to the dispense position of the outer cover without any intermediate position.

33. The medicament dispenser according to claim 30, wherein the dispensing of the medicament provides continuous movement of the outer cover from the initial position to rotate the lever and actuate the medicament carrier advancing mechanism.

34. The medicament dispenser according to claim 30, wherein the dispensing of the medicament provides continuous movement of the outer cover from the initial position results in actuation of the medicament carrier advancing mechanism.

35. The medicament dispenser according to claim 30, wherein the dispensing of the medicament does not provide any idle movement of the outer cover.
